(19) United States
(12) Patent Application Publication
(10) Pub. No.: US 2021/0076724 A1
(43) Pub. Date: Mar. 18, 2021

(54) ENZYMATIC IN-SITU FORTIFICATION OF FOOD WITH FUNCTIONAL CARBOHYDRATES

(71) Applicant: C-LEcta GmbH, Leipzig (DE)

(72) Inventors: Andreas BUTHE, Leipzig (DE); Martina BLUHM, Leipzig (DE); Marc Struhalla, Leipzig (DE)

(73) Assignee: C-LEcta GmbH, Leipzig (DE)

(21) Appl. No.: 16/971,378

(22) PCT Filed: Feb. 27, 2019
(86) PCT No.: PCT/EP2019/054904

§ 371 Date: Aug. 20, 2020

(30) Foreign Application Priority Data
Feb. 28, 2018 (EP) 18159317.9
Mar. 23, 2018 (EP) 18163642.4

(51) Int. Cl. A23L 33/125; A23C 9/12; A23C 9/123; A23L 2/02; A23L 2/84; A23C 9/13; A23L 2/52; A23L 27/30

(52) U.S. Cl. CPC A23L 33/125; A23C 9/1206; A23C 9/1216; A23C 9/123; A23V 2002/00; A23L 2/84; A23C 9/1307; A23L 2/52; A23L 27/33; A23L 2/02

(57) ABSTRACT

The invention relates to the enzymatic treatment of a virgin liquid nutrient naturally containing carbohydrates for the in-situ production of functional carbohydrates, thereby obtaining a fortified processed liquid nutrient, being rich (or enriched) in such functional carbohydrates and offering a beneficial nutritional value. The invention relates to the in-situ use of enzymes during food processing of a virgin liquid nutrient for the preparation of fortified food containing supplementary functional carbohydrates of specified composition.

The invention relates to the enzymatic treatment of a virgin liquid nutrient naturally containing carbohydrates for the in-situ production of functional carbohydrates, thereby obtaining a fortified processed liquid nutrient, being rich (or enriched) in such functional carbohydrates and offering a beneficial nutritional value. The invention relates to the in-situ use of enzymes during food processing of a virgin liquid nutrient for the preparation of fortified food containing supplementary functional carbohydrates of specified composition.

Due to a steadily growing awareness of consumers worldwide in respect to the safety and benefits of food, opportunities to increase the nutritional values and/or health benefits of food products without significantly impacting the organoleptic properties of food products is highly desirable. This can be achieved either by the modification of the food itself or by admixing functional ingredients.

In particular, a number of natural functional carbohydrates have already been identified and characterized for their health and technical functionalities in foodstuff. These functional carbohydrates can be obtained either by extraction from food or in manufacturing processes using enzymes or whole-cell catalysts. Manufacturing processes have in common that widespread and naturally occurring carbohydrates are used as substrates. Eligible substrates are disaccharides like sucrose, lactose and maltose, monosaccharides like glucose, fructose and galactose as well as oligo- and polysaccharides like for example, but not limited to, inulin, starch, maltodextrin, xylan, pectin, arabinan, arabinoxylan, arabinogalactan, and cellulose. Some carbohydrate-based ingredients are classified as prebiotic and/or low glycemic carbohydrates and are used to promote a healthy gut microbiome and/or to prevent diabetes. Some carbohydrate-based ingredients are not metabolized like conventional sugars and hence having a lower calorie count, likewise contributing to consumer's health.

Most of the naturally prevailing mono- and disaccharides are responsible for the sweetness and high calorie count of foodstuff. However, there is a high interest of consumers in food that delivers the desired sweetness and textural sensation but concomitantly a low calorie count. Consequently, a number of light or diet products respectively were introduced to the market containing non-natural high intensity sweeteners. More recently, natural high intensity sweeteners like steviol glycosides or mogrosides were launched. However, at present non-natural sweeteners like aspartame, cyclamate and sucralose are still prevailing, which are used in combination with taste modifiers to create a sweetness profile close to sucrose as much as possible. Besides being non-natural, another drawback of non-natural sweeteners is given by their high intensity and in turn the only low amounts applicable in foodstuff, lacking the bulking properties of conventional carbohydrates. This drawback reflects the fact that caloric (sweetening) carbohydrates like sucrose, lactose, glucose, fructose, and galactose also provide an important technical functionality as they have a strong impact as bulking agent on the organoleptic properties and thus on the resulting textural sensation. Even though desirable for a low calorie count, their substitution is hampered by a loss of said technical functionalities and therefore limits applicability of high intensity sweeteners to certain foodstuff like beverages. In other foodstuff the use of high intensity sweetener would require the use of additional functional but low caloric ingredients that provide texture. One example in this respect is the prebiotic natural polysaccharide inulin that is used as additive to provide texture in food (Roberfroid 2007, Meyer et. al. 2011).

Alternatives to high-intensity are low-intensity sweeteners like for example tagatose, xylitol, erythritol, trehalose, isomaltulose, and allulose. Low intensity-sweeteners have sweetness comparable to sucrose and can be used in similar amounts, thus impacting organoleptic and textural properties likewise comparable to sucrose. Even though some low-intensity sweeteners like isomaltulose or trehalose have the same calorie count as sucrose their use is still deemed highly beneficial as they are digested slowly and steadily, what accounts for a low glycemic index (Maresch et. al. 2017, Yoshizane 2017). Sweeteners like the natural monosaccharide D-allulose (also known as D-psicose) or D-tagatose, also receive great attention. D-allulose has about 92% of the relative sweetness of sucrose, comparable to glucose, but only provides 0.2 kcal/mol energy corresponding to a calorie count that is 90% lower (Chung et. al. 2011: Hypoglycemic Health Benefits of D-Psicose; J Agric Food Chem.; 60(4):863-9). Its sweetness profile is very similar to glucose in regards to intensity and sweetness. However, the body metabolizes D-allulose differently than sugars such as glucose and fructose resulting in a significantly lower calorie count. The sweetness of D-tagatose is 70% of the sweetness of sucrose, while the calorie count is with 1.5 kcal/g only 40% of sucrose (Kirk-Othmer, Chapter 3.2 in Food and Feed Technology, 2 Volume, Wiley). Additionally, several health benefits are claimed for D-allulose, D-tagatose, and isomaltulose including improved insulin resistance, antioxidant enhancement and formation, and hypoglycemic controls.

The monosaccharide D-mannose is used as therapeutic prophylaxis of bladder infections (cystitis) and available in the market as dietary supplement (Altarac and Papeš 2014: Use of D-mannose in prophylaxis of recurrent urinary tract infections (UTIs) in women; BJU International, Vol. 113(1): 9-10). There are several other health benefits accounted for D-mannose if used in nutrition (Hu et al. 2016: D-mannose: Properties, Production, and Applications: An Overview. Compr Reviews in Food Science and Food Safety, Vol. 15(4): 773-785). Disaccharides like trehalose, cellobiose, and kojibiose are considered as technical sugars, low-glycemic sugars and/or prebiotic sugars (Clemens et al. 2016: Functionality of Sugars in Foods and Health; Comprehensive Reviews in Food Science and Safety, Vol. 15(3): 433-470, Basholli-Salihu et. al. 2013; Luz Sanz et. al 2005). Difructose anhydrides are composed of two fructose units and are appreciated for their very low calorie count and for various health benefits (Ortiz-Mellet et. al. 2010: Carbohydrates in Sustainable Development, page 49-77).

An overview of some common and emerging functional carbohydrates and associated benefits as well as enzymes being used for the preparation thereof, which are well-known to people skilled in the art, is provided by Table 2 and Table 3. Selection was guided by enzymatic accessibility of said functional carbohydrates (altered carbohydrates) via carbohydrate feedstocks (initial carbohydrates) that occur naturally in milk and fruit. Therefore, besides the functional carbohydrates that are subject matter of the invention also functional carbohydrates like GOS, FOS, lactulose, and epilactose are enlisted. For example, lactose as such is also the carbohydrate feedstock for the enzymatic production of galacto-oligosaccharides (GOS), also known as oligogalactosyllactose, oligogalactose, oligolactose or transgalactooligo-saccharides. GOS can be produced by the enzyme lactase, also known as beta-galactosidase, as long as such lactase has a transgalactosylation activity. A transgalactosylation is defined as the addition of galactosyl units from lactose onto lactose, galactose, or existing galacto-oligomers to form oligomers. Because GOS are non-digestible carbohydrates that pass the small intestine and thereby selectively promote the growth of bifidobacteria and other beneficial intestinal flora associated with numerous health benefits (Hughes et al. (1991), Food Technol., 45: 64-83), GOS have been suggested to be used in a number of different food applications. Indeed, it is mainly used to fortify infant food by admixing. In a conventional manufacturing process, GOS are produced in form of syrup by using high concentrations of lactose as substrate in an enzymatic in-vitro process. The transgalactosylation activity of the used enzymes is concentration-dependent and works best in concentrated solutions of lactose (EP2130438).

The applicability and safety of D-allulose, trehalose, D-tagatose and isomaltulose is proven by being admixed to a number of commercially available products like beverages, yogurt, ice cream, baked goods, and other food items. Thus, the relevance of these functional carbohydrates is obvious due to the availability of such functional carbohydrate containing products. The use of other functional carbohydrates like cellobiose and kojibiose is emerging, but as they are considered to have highly promising properties, a broader application in future time can be expected. Consequently, WO 2016/038142 discloses a process for the manufacturing of cellobiose and WO 2016/116627, WO 2016/116622, WO 2016/116619, and WO 2016/116620 its use in food relevant applications. And also for kojibiose, production processes are an ongoing matter of development. Due to the highly stable alpha-1,2 glycosidic bond kojibiose is considered to be a highly potent prebiotic what was shown in scientific studies (WO 2016/075219).

D-Allulose is commercially produced by using D-psicose 3-epimerase (EC 5.1.3.30) or D-tagatose 3-epimerase (EC 5.1.3.31) to convert D-fructose to D-allulose. Fructose can be obtained from various sources e.g. the disaccharide sucrose or the polysaccharide starch, if hydrolyzed into the constituting monosaccharide glucose, which then gets isomerized to fructose by the use of the enzyme glucose isomerase. Comprehensive prior art is disclosed, e.g. in WO 2016/160573 and US 2015/0210996.

D-Tagatose is commercially produced by using L-arabinose isomerase (EC 5.3.1.4) to convert D-galactose to D-tagatose. Galactose can be obtained by the hydrolysis of lactose. Comprehensive prior art is disclosed, e.g. in WO 2008/066280 and EP 3115453. U.S. Pat. No. 6,057,135 for example describes the manufacturing of D-tagatose out of galactose that is obtained from cheese whey and/or milk. The cheese whey and/or milk is hydrolyzed to prepare a mixture comprising galactose and glucose. Galactose is then separated from the glucose by fermentation and subjected to isomerization using said L-arabinose isomerase.

D-Mannose can be produced by using an enzyme like cellobiose-2-epimerase (EC 5.1.3.11) to convert D-glucose into D-mannose. Glucose can be obtained by the hydrolysis of lactose, sucrose or polysaccharides like starch or cellulose. Prior art is disclosed in literature (Park et al. (2011): Characterization of a recombinant cellobiose 2-epimerase from Caldicellulosiruptor saccharolyticus and its application in the production of mannose from glucose. Appl Microbiol Biotechnol., Vol. 92(6): 1187-96). D-Mannose can also be produced by using an enzyme like mannose isomerase (EC 5.1.3.7) to convert D-fructose into D-mannose. Fructose can be obtained by the hydrolysis of sucrose using an invertase (or beta-fructofuranosidase, EC 3.2.1.26), or by hydrolysis of oligo- and polysaccharides composed of fructose (fructans) using the respective hydrolytic enzymes. Fructose can also be obtained by the isomerization of glucose using an isomerase (EC 5.3.1.5). Prior art is disclosed in literature (Hu et al. (2016): D-mannose: Properties, Production, and Applications: An Overview. Compr Reviews in Food Science and Food Safety, Vol. 15(4): 773-785).

Isomaltulose is manufactured by enzymatic rearrangement (isomerization) of sucrose using the enzyme isomaltulose synthase (EC 5.4.99.11). Comprehensive prior art is disclosed, e.g. in EP 0028900 and EP 2704594.

Trehalose can be produced by using a sucrose phosphorylase (EC 2.4.1.7) that converts sucrose and inorganic phosphate into glucose-1-phosphate and fructose. By using a trehalose-phosphorylase (EC 2.4.1.64) glucose-1-phosphate gets transferred to a second glucose moiety under formation of an alpha-1,1-glycosidic bond. If sucrose is the only carbohydrate substrate, the additional glucose can be made available by using a glucose isomerase to convert the initially released fructose into glucose. Some prior art is disclosed, e.g. in EP 0677587. An alternative process using starch as substrate is disclosed in EP 0693558.

Cellobiose can be produced by using a sucrose phosphorylase (EC 2.4.1.7) that converts sucrose and inorganic phosphate into glucose-1-phosphate and fructose. By using a cellobiose-phosphorylase (EC 2.4.1.20) glucose-1-phosphate gets transferred to a second glucose moiety under formation of an beta-1,4-glycosidic bond. If sucrose is the only carbohydrate substrate, the additional glucose molecules can be made available by using a glucose isomerase to convert the initially released fructose into glucose. Some prior art is disclosed, e.g. in WO 2016/038142. An alternative process using cellulose as substrate is disclosed in EP2402454.

Kojibiose can be produced by using a sucrose phosphorylase that—at low concentrations of inorganic phosphate but in the presence of glucose—transfers the glucose moiety of sucrose to another (free) glucose molecule under formation of a alpha-1,2-glycosdic bond. The same principle can be applied for the synthesis of nigerose if, depending on the specific sucrose phosphorylase, a alpha-1,3-glycosidic bond is formed. Some prior art is disclosed in WO 2016/075219.

The difructose anhydride alpha-D-fructofuranose beta-D-fructofuranose 1,2′:2,3′-dianhydride (DFA III) can be produced by using an inulin-fructotransferase (EC 4.2.2.18) that cleaves of the terminal D-fructosyl-D-fructosyl from inulin under formation of the anhydride. Comprehensive prior art is disclosed, e.g. in EP 1282715.

Isomalto-oligosaccharides (also referred to as “IMO” or “IMOs”) are oligomers of glucose subunits being connected with alpha-D-(1,6)-linkages predominantly, but may also contain other linkages like for example alpha-D-(1,4)-linkages. They include oligosaccharides like isomaltose, isolmaltotriose, isomaltotetrose or isomaltopentose. IMOs can be produced from starch by use of certain enzymes: α-amylase (EC 3.2.1.1) is used to liquefy starch while alpha-amylase and beta-amylase (EC 3.2.1.2) and a pullulanase (EC 3.2.1.41) are used for saccharification to form syrup comprising maltose and maltotriose, which is followed by the use of an alpha-transglucosidase (EC 2.4.1.24) to form IMOs. The enzymatic IMO formation is described e.g. in U.S. Pat. No. 8,715,755B2. Optionally, IMOs can be produced from mixtures of sucrose and maltose substrates by use of an alpha-transglucosidase (EC 2.4.1.24). IMOs can be also produced from sucrose by use of a glucansucrase, preferably a dextransucrase enzyme (EC 2.4.1.5). Such enzymatic IMO formation is described e.g. in WO 2004/068966 and Tanriseven & Dogan 2002 (Production of isomalto-oligosaccharides using dextransucrase (EC 2.4.1.5) immobilized in alginate fibres. Process Biochemistry, Vol. 37(10), 1111-1115). Isomaltose can be produced from Sucrose by use of dextransucrase and dextranase (EC 3.2.1.11, EC 3.2.1.94) enzymes as described in U.S. Pat. No. 4,861,381.

Gluco-oligosaccharides (also referred to as “GlucOS” or “GlucOSs”) are oligomers of glucose subunits being connected with mixtures of different linkage types (alpha-D-(1,2)-linkages, alpha-D-(1,3)-linkages, alpha-D-(1,4)-linkages, and alpha-D-(1,6)-linkages). The abbreviation “GlucOS” is more meaningful than the abbreviation “GOS”, because the latter is commonly used also to refer to galacto-oligisaccharides. According to C. Geissler et al., Human Nutrition, 12th ed. Elsevier 2011, a gluco-oligosaccharide is a non-digestible oligosaccharide of glucose containing alpha-1,2 and alpha-1,6 glycosidic links. The group of GlucOS usually includes smaller oligosaccharides starting from tri-saccharide sizes up to deca-saccharides. Examples of GlucOS are, without limitation, isomaltotriose, isomaltotetraose, kojitriose, kojitetraose. GlucOS can be produced enzymatically from sucrose, optionally in the presence of maltose, by use of a glucansucrase (EC 2.4.1.5; EC 2.4.1.140), and/or a dextransucrase (EC 2.4.1.5).

All known technical processes described in the state of the art for the preparation of functional carbohydrates, in particular for the preparation of D-allulose, D-tagatose, D-mannose, isomaltulose, trehalose, cellobiose, IMOs, GlucOS, and kojibiose, are aiming at the manufacturing of a highly enriched and pure product that can be used as a defined and approved food ingredient, which afterwards is admixed with other components in food manufacturing. These processes are operated under conditions that enable highest space time yields and make use of deliberately selected processing aids and reaction conditions. Among those processing aids are the enzymes that are used as biocatalysts, and which are typically engineered to assure that the enzymes cope with the specific reaction conditions encountered during the process, for instance in terms of substrate/product concentrations, pH, and temperature. Substrates for the manufacturing of these functional carbohydrates are applied as rather pure substrates, or mixtures from such pure substrates, wherein the pure substrates have been obtained by industrial processing of liquid milk or agricultural crops like for example, but not limited to, sugar cane, sugar beet, corn, pea, and, potatoes.

Typically, food ingredients are admixed prior to or during the manufacturing and/or preparation of food, yielding a processed food product characterized by a list of ingredients. However, due to the steadily growing health awareness of many consumers, there are trends like clean(er) eating aiming at the predominant consumption of non-processed foods (like fruits and vegetables) or low-processed foods (gently processed food but with no or a minimum of ingredients like plain yoghurts or dry jerky). In addition, there is an interest in natural and healthy foodstuff, characterized by significant amounts of beneficial natural ingredients while at the same time obtained by a moderate processing. Besides, the growing numbers of people suffering from obesity and associated diseases such as diabetes and cardiovascular diseases, which result from a high sugar/high calorie intake, are a major health concern. However, dietary food is often perceived as non-natural and not well-accepted by many consumers.

Therefore, there is a demand for low-processed food products that would benefit from being fortified with valuable, but natural functional carbohydrate ingredients with a reduced calorie count and reduced glycemic index, but at the same time being considered as low-processed food with the typical organoleptic properties expected by the consumer. This demand is especially prevalent for dairy products or for liquid foodstuff obtained e.g. by the extraction of fruits or vegetables. However, such products would only be available, if the fully-caloric carbohydrates (initial carbohydrates) as naturally present in the respective raw materials are extracted thereof in a first processing step and substituted by said functional carbohydrate ingredients in a second processing step. To avoid such intense processing steps, which might negatively affect other healthy but sensitive ingredients, the enzymatic in-situ conversion of naturally contained carbohydrates into functional and beneficial carbohydrates would offer a gentle possibility to fortify foodstuff without being subject to heavy processing.

In case of dairy products and the herein contained lactose, the concept of enzymatic in-situ fortification was already applied in various forms: lactose is the prevailing sugar found in untreated dairy products and its digestion requires a hydrolytic enzyme (lactase/beta-galactosidase) to split the disaccharide into the respective (high glycemic) monosaccharides galactose and glucose, which are then readily absorbed into the bloodstream. This enzyme is naturally secreted in the intestine. Lactase is generally produced in large amounts at birth and in early childhood when milk is consumed as a primary part of the diet. However, diminishing levels of this enzyme result in an incomplete digestion and adversely affect the consumer's well-being. For consumers suffering from this lactose-intolerance the consumption of lactose-free dairy products is one alternative and the use of beta-galactosidases to produce lactose-free milk is a well-known form of in-situ modification. However, in-situ modified lactose-free milk still contains some lactose (e.g. approx. 0.5% besides approx. 2% glucose and galactose each) but these levels are not affecting lactose-intolerant consumers (Pirisino J. F. 1983: High Performance Liquid Chromatographic Determination of lactose, glucose, and galactose in lactose-Reduced Milk; Food Science Vol. (48)3: 742-744).

Beta-Galactosidase-processed dairy products have been introduced to the market a long time ago. However, it is frequently reported that the taste of such products is perceived as less pleasant, mainly due to higher relative sweetness of the monosaccharides versus the disaccharide each in comparison to sucrose (0.6-0.7 for glucose and 0.5 to 0.7 for galactose in relation to sucrose versus 0.2-0.4 of lactose in relation to sucrose) (EP2130438; Schaafsma, G. (2008): lactose and lactose derivatives as bioactive ingredients in human nutrition. International Dairy Journal, 18(5), 458-465). Another drawback associated with the preparation of lactose-free milk is the deterioration of the nutritional value since the hydrolysis of lactose results in an increase of the calorie count and glycemic index. Thus, dairy products having reduced lactose levels and taste similar to full lactose products would be very desirable for many consumers (EP2130438).

Other attempts to provide the consumers with lactose-reduced dairy products based on the use of a beta-galactosidase in liquid milk to produce in-situ galacto-oligosaccharides (GOS). WO 2015/132402 discloses a method of producing milk products containing GOS at low temperature using beta-galactosidase enzymes having a high level of transgalactosylation activity. EP2130438 discloses processes dealing with the production of cream cheese products containing GOS and having significantly reduced lactose levels after being contacted with lactase enzyme(s) having hydrolytic and transgalactosylation activities. However, the taste of such product is still off the consumer's expectation due to a loss of sweetness. US 20110117243 discloses the in-situ use of a galactosidase and a glucose isomerase to increase the sweetness of whey derived products by the formation of fructose. Even though taste might be improved, the calorie count remains the same while some nutritional drawbacks can be ascribed to the formed fructose despite the lower glycemic index (Feinman and Fine (2013): fructose in perspective. Nutrition & Metabolism, Vol. 10(45): 2-11).

As another modification of liquid milk, EP2395080 describes the use of a cellobiose-2-epimerase in liquid milk to in-situ generate the non-natural prebiotics lactulose and epilactose with a minor relative sweetness compared to sucrose.

Other processes describe the enzymatic treatment of liquid foodstuff derived from fruits or vegetables: U.S. Pat. No. 8,168,242 describes the use of enzymes in fruit juice to produce in-situ fructans what is accompanied by a major loss of sweetness. EP 1167536 describes the in-situ use of pectinases to obtain L-arabinose by degradation of structural polysaccharides in liquids obtained from vegetables. The degradation of pectin may affect texture, the release of D-arabinose as such accounts for an increase of calorie count and glycemic index.

With regards to the naturally contained carbohydrates lactose or sucrose all of this prior art has in common that the initial carbohydrates are transformed into prebiotic primarily oligomeric and polymeric carbohydrates. However, the resulting carbohydrates provide a product with significantly altered organoleptic properties due to a reduced relative sweetness and a modified texture as the disaccharides are converted into oligosaccharides. Even though, the calorie count and glycemic index of such products is reduced, the altered organoleptic properties adversely affect the consumer's acceptance.

WO 2015/036637 relates to a method for the synthesis of kojibiose using a starting reaction mixture containing, as the main compound thereof, the trisaccharide 2-α-D-glucopyranosyl-lactose, O-β-D-galactopyranosyl-(1→4)-O-[α-D-glucopyranosyl-(1→2)]-β-D-glucopyranose, and leucrose, lactose, fructose, glucose and saccharose, said method comprising steps of fermentation by use of a microbial strain, followed by enzymatic hydrolysis treatment and purification of the mixture, allowing the production of a disaccharide with high added value, such as kojibiose, in a cost-effective manner.

Kitao et al., Bioscience Biotechnology Biochemistry, vol. 58, no. 4, 1994, 790/791 relates to the formation of kojibiose and nigerose by sucrose phosphorylase.

Chean et al., Journal of Bioscience and Bioengineering, vol. 92, no. 2, 2001, 177-182 relates to the enzymatic synthesis of kojioligosaccharides using kojibiose phosphorylase;

WO 2017/081666 provides a process for preparing non-cariogenic, sustained energy release juice. The process comprises contacting juice with an enzyme immobilized on Duolite at 30-50° C. for 1-5 h; wherein the enzyme is capable of converting cariogenic sugar to non-cariogenic sugar; and separating juice from the enzyme complex.

WO 2017/059278 provides a process for enzymatically converting a saccharide into tagatose. The process involves converting fructose 6-phosphate (F6P) to tagatose 6-phosphate (T6P), catalyzed by an epimerase, and converting the T6P to tagatose, catalyzed by a phosphatase.

EP 2 130 438 discloses processes directed to cream cheese products containing galacto-oligosaccharides and having significantly reduced lactose levels. More specifically, lactose-containing dairy substrates are contacted with lactase enzyme(s) having hydrolytic and trans-galactosylation activities effective for converting at least 20 percent of the lactose in the dairy substrate to galacto-oligosaccharides. The enzyme-treated dairy substrate is then processed into galacto-oligosaccharide containing cream cheese products having reduced lactose levels.

Cantarella et al., Enzyme and Microbial Technology, vol. 15, no. 5, 1994, 383-387 relates to disaccharide production by glucoamylase in aqueous ether mixtures.

Database FSTA, International Food Information Service, vol. 69, no. 12, 1974, 841-843 relates to oligosaccharide formation from steamed rice in the presence of maltose and alcohol by an Aspergillus enzyme.

WO 03/020054 relates to a beverage in which difructose dianhydride III is added as prebiotic dietary fibers, wherein the DFA III shows, even at a pH value of <3.9, sufficient storage stability during the entire shelf life of the beverage.

EP 0 332 108 discloses a process for preparing difructose dianhydride III (DFA III) comprising reacting inulin with an inulin lytic enzyme derived from a microorganism belonging to Arthrobacter ilicis. The employed enzyme efficiently produces DFA III from inulin and is more stable against heat than conventional enzymes. The process enables industrial continuous production of DFA III. The preferred strain is Arthrobacter ilicis MCI 2297 (FERM P-9893).

The methods for the treatment of nutrients and the nutrients of the prior art are not satisfactory in every respect and there is a demand for improved methods and improved nutrients. It is an object of the invention to provide methods that have advantages compared to the prior art.

This object has been solved by the subject-matter of the patent claims, the description, the examples, and the figures.

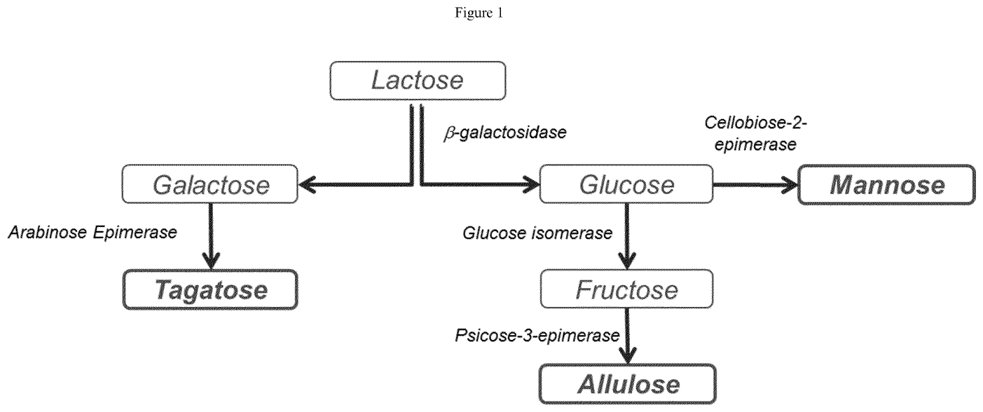

FIG. 1 shows the reaction scheme for the manufacturing of D-tagatose, D-allulose, and D-mannose as functional carbohydrates (altered carbohydrates) by enzymatic in-situ processing out of lactose, galactose and/or glucose (initial carbohydrates), which are naturally contained in milk-based virgin liquid nutrients. The depicted reaction patterns are also applicable if the initial carbohydrates are admixed as ingredients to a food preparation, or when mixing different virgin liquid nutrients, or when supplementing virgin liquid nutrients from external source.

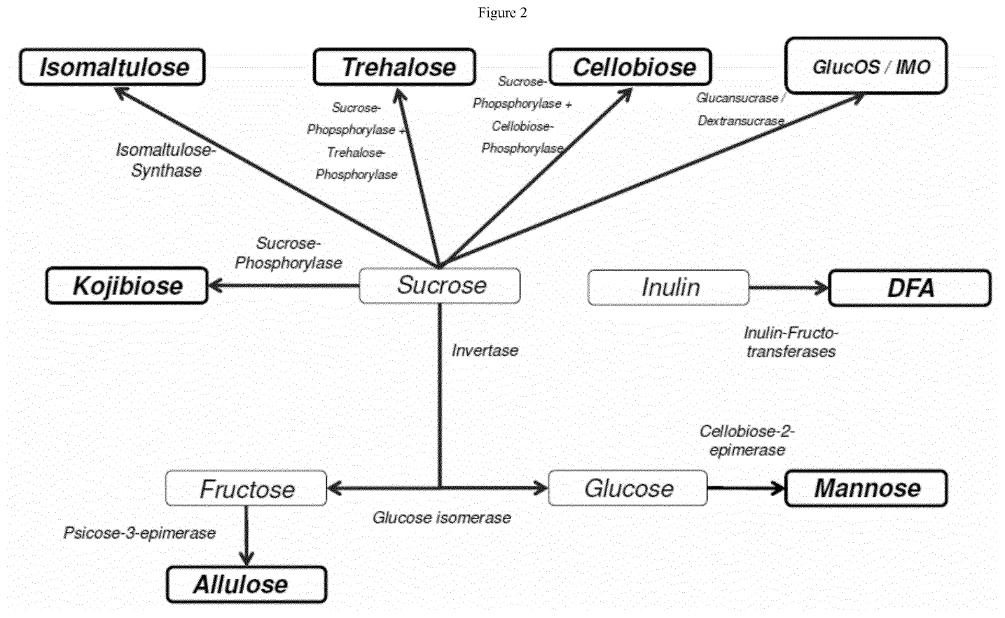

FIG. 2 shows the reaction scheme for the manufacturing of isomaltulose, trehalose, cellobiose, kojibiose, nigerose, IMOs, GlucOS, D-allulose, and D-mannose as functional carbohydrate ingredients (altered carbohydrates) by enzymatic in-situ processing out of sucrose, fructose and/or glucose (initial carbohydrate), naturally contained in virgin liquid nutrients in form of extracted fruit juice. Difructose anhydride (DFA III, altered carbohydrate) can be produced out of inulin (initial carbohydrate), naturally contained in certain virgin liquid nutrients in form of extracted fruit juice. The depicted reaction patterns are also applicable if the initial carbohydrates are admixed as ingredients to a food preparation, or when mixing different virgin liquid nutrients, or when supplementing virgin liquid nutrients from external source.

Unless expressly stated otherwise, all carbohydrates can be present in the D-form, the L-form or any mixture thereof in any ratio. Preferably, the carbohydrates are present in the D-form.

Unless expressly stated otherwise, the term “IMO” preferably refers to oligomers of glucose subunits being connected with alpha-D-(1,6)-linkages selected from the group consisting of isomaltose, isolmaltotriose, isomaltotetrose or isomaltopentose.

Unless expressly stated otherwise, the term “GlucOS” (or gluco-oligosaccharides) preferably refers to oligomers of glucose subunits being connected with mixtures of different linkage types (alpha-D-(1,2)-linkages, alpha-D-(1,3)-linkages, alpha-D-(1,4)-linkages, and alpha-D-(1,6)-linkages) and selected from the group consisting of tri-saccharides, tetra-saccharides, penta-saccharides, hexa-saccharides, hepta-saccharides, octa-saccharides, nona-saccharides, and deca-saccharides. Preferably, at least one subunit within the oligomer is a glucose subunit, more preferably all subunits within the oligomer are glucose subunits.

In a first aspect, the invention relates to a method for the enzymatic in-situ processing of a virgin liquid nutrient comprising one or more initial carbohydrates into a processed liquid nutrient, the method comprising the steps of
(i) providing a virgin liquid nutrient which comprises
   one or more initial carbohydrates; preferably selected from the group consisting of sucrose, inulin, lactose, glucose, galactose, starch, maltose, and fructose; and
   preferably, one or more additional ingredients selected from the group consisting of lipids, proteins, vitamins, metabolites, colloids or colloidal particles, phytochemicals, fibers, and polysaccharides other than starch;
(ii) optionally, adjusting (ii-a) pH value and/or (ii-b) temperature of the virgin liquid nutrient,
(iii) optionally, supplementing (iii-a) inorganic phosphate and/or (iii-b) cofactors such as
   salts of metal cations (e.g. Fe²⁺, Fe³⁺, Mg²⁺, Mn²⁺, Mn³⁺, Ca²⁺, Co²⁺, Co³⁺, Cu²⁺, Zn²⁺, or Mo²⁺) that are soluble in the virgin liquid nutrient to the virgin liquid nutrient; and/or
   ATP, ADP, NAD, NADP, FAD, pyridoxal phosphate, tetrahydrofolic acid, cobalamine, ascorbic acid, coenzyme A, coenzyme Q10, or alpha-liponic acid; and/or
   (iii-c) one or more initial carbohydrates; and
(iv) treating the virgin liquid nutrient with one or more enzymes, thereby converting at least a portion of the at least one initial carbohydrate into one or more altered carbohydrates and thus obtaining the processed liquid nutrient,
   wherein the processed liquid nutrient is preferably characterized by
   a glycemic index of all (residual) initial carbohydrates and altered carbohydrates contained in the processed liquid nutrient, which is lower than the glycemic index of all initial carbohydrates contained in the virgin liquid nutrient; and/or
   a calorie count of all (residual) initial carbohydrates and altered carbohydrates contained in the processed liquid nutrient, which is lower than the calorie count of all initial carbohydrates contained in the virgin liquid nutrient; and/or
   a textural sensation conferred by all (residual) initial carbohydrates and altered carbohydrates contained in the processed liquid nutrient, which is connatural compared to the textural sensation conferred by all initial carbohydrates contained in the virgin liquid nutrient; and/or
   a sweetness conferred by all (residual) initial carbohydrates and altered carbohydrates contained in the processed liquid nutrient, which is connatural compared to the sweetness conferred by all initial carbohydrates contained in the virgin liquid nutrient.

Thus, the starting material that is employed in the method according to the invention is a virgin liquid nutrient comprising one or more initial carbohydrates, whereas the product that is obtained by the method according to the invention is a processed liquid nutrient comprising one or more altered carbohydrates. The one or more altered carbohydrates are obtained by enzymatic conversion from at least a portion of the one or more initial carbohydrates contained in the virgin liquid nutrient (starting material).

It is contemplated that said one or more altered carbohydrates may already be contained in an initial amount besides said one or more initial carbohydrates in the virgin liquid nutrient (starting material) such that the enzymatic conversion results in an enrichment of said one or more altered carbohydrates in the processed liquid nutrient. The total quantity of one or more altered carbohydrates in the processed liquid nutrient is then the combination of the initial amount already contained in the virgin liquid nutrient with the additional amount obtained by enzymatic conversion. In preferred embodiments of the method according to the invention, at least 20 wt.-%, or at least 30 wt.-%, or at least 40 wt.-%, preferably at least 50 wt.-%, more preferably at least 60 wt.-%, still more preferably at least 70 wt.-%, yet more preferably at least 80 wt.-%, even more preferably at least 90 wt.-%, most preferably at least 95 wt.-%, and in particular at least 99 wt.-% of the total amount of said one or more altered carbohydrates contained in the processed liquid nutrient (product) were not originally contained in the virgin liquid nutrient (starting material).

Thus, for the purpose of the specification, the term “altered” does not necessarily mean that a given molecule has been actually altered by enzymatic conversion, but merely qualitatively distinguishes the chemical nature of the reaction products from the chemical nature of the starting materials. The total quantity of altered carbohydrates in the processed liquid nutrient encompasses any fraction thereof that was already initially contained in the virgin liquid nutrient and that therefore has not been obtained by enzymatic conversion.

It is further contemplated that said one or more initial carbohydrates in the virgin liquid nutrient (starting material) may be contained in the naturally occurring amount without any external supplementation. For example, when the virgin liquid nutrient is apple juice, 100 ml of a representative apple juice may have an exemplified content of 1.9 g glucose, 5.3 g fructose and 2.4 g sucrose. It is contemplated that in the course of the method according to the invention, each of the one or more initial carbohydrates in the virgin liquid nutrient is subject to enzymatic conversion into one or more altered carbohydrates in the processed liquid nutrient. It is also contemplated that in the course of the method according to the invention, only one or more initial carbohydrates in the virgin liquid nutrient, but preferably not all initial carbohydrates in the virgin liquid nutrient, are subject to enzymatic conversion into one or more altered carbohydrates in the processed liquid nutrient. For example, when the fructose contained in apple juice is enzymatically converted into an altered carbohydrate, the glucose and sucrose that are additionally contained in said apple juice remain unaffected. Alternatively, when the fructose contained in apple juice is enzymatically converted into an altered carbohydrate and when in parallel, i.e. in a second enzymatic conversion, glucose is isomerized into fructose and subsequently also enzymatically converted into said altered carbohydrate, only the sucrose that is additionally contained in said apple juice remains unaffected.

It is further contemplated that enzymatic conversion may be essentially complete (i.e. provides a conversion yield of about 100%) such that essentially the total amount of the one or more initial carbohydrates subject to enzymatic conversion and originally contained in the virgin liquid nutrient is enzymatically converted into said one or more altered carbohydrates in the processed liquid nutrient. Under these circumstances, the processed liquid nutrient essentially comprises no residual amounts of said one or more initial carbohydrates subject to enzymatic conversion and originally contained in the virgin liquid nutrient. Typically, however, enzymatic conversion provides yields below 100%, such that the processed liquid nutrient besides the altered carbohydrates also comprises residual amounts of said one or more initial carbohydrates originally contained in the virgin liquid nutrient that are principally subject to enzymatic conversion, but because of the conversion yield below 100% were not enzymatically converted. Specifically, enzymatic conversions often provide yields below 100%, wherein the yields correspond to the specific thermodynamic equilibrium of the enzymatic conversion under the given conversion conditions.

It is also contemplated that said one or more initial carbohydrates subject to enzymatic conversion and originally contained in the virgin liquid nutrient (starting material) may be enriched by supplementation from an external source from which at least a portion becomes subject to enzymatic conversion into one or more altered carbohydrates. For example, when the fructose contained in apple juice is enzymatically converted into an altered carbohydrate, the natural content of fructose in apple juice (e.g. 5.3 g in 100 ml) may be supplemented by additional fructose from an external source. It is also contemplated that said one or more initial carbohydrates subject to enzymatic conversion and are not originally contained in the virgin liquid nutrient, but are introduced as starting material to a virgin liquid nutrient by supplementation from an external source from which at least a portion becomes subject to enzymatic conversion into one or more altered carbohydrates. For example, when liquid milk is supplemented with fructose, originally not contained in liquid milk, such fructose may be enzymatically converted into an altered carbohydrate, for example into D-allulose.

While said external source may principally be any source including essentially pure e.g. crystalline carbohydrate, in preferred embodiments of the invention said external source is a carbohydrate composition such as honey or syrup, wherein syrup is preferably derived from starch, grain, rice or vegetable processing (e.g. high fructose corn syrup, rice syrup, grain syrup, barley syrup, or the like). A skilled person recognizes that such carbohydrate compositions are typically complex mixtures as such. Therefore, when supplementing the one or more initial carbohydrates subject to enzymatic conversion and originally contained in the virgin liquid nutrient (starting material) by enrichment from an external source, additional initial carbohydrates that are not subject to enzymatic conversion may simultaneously be supplemented and/or enriched in the virgin liquid nutrient.

For example, in a preferred embodiment of the method according to the invention, the virgin liquid nutrient comprises liquid milk, wherein step (iii-3) involves supplementing fructose as initial carbohydrate, and wherein step (iv) involves the enzymatic conversion of at least a portion of the fructose into D-allulose. In a more preferred example, the liquid milk is yogurt.

Irrespective of the above variations of the method that are all in accordance with the present invention, it is essential that the method according to the invention is an enzymatic in-situ conversion. The enzymatic conversion takes place within the virgin liquid nutrient where at least a portion of one or more initial carbohydrates subject to enzymatic conversion is enzymatically converted into one or more altered carbohydrates thereby providing the processed liquid nutrient. The in-situ conversion according to the invention typically proceeds in the presence of numerous other ingredients that are originally contained in the virgin liquid nutrient but that are not subject to enzymatic conversion and thus are also contained in the same quantity in the processed liquid nutrient (additional ingredients).

This is unconventional because many enzymes are sensitive and require distinct reaction conditions such as specific concentrations of substrates, cofactors, temperature and pH value in order to provide satisfactory conversion yields. Therefore, most industrial processes involving enzymatic conversions are performed under extensive control of reaction conditions in highly pure media only containing ingredients that are absolutely necessary for the desired enzymatic conversion. The presence of any additional ingredients that are not required for enzymatic conversion or that might have a negative impact on enzymatic conversion or that are even unknown due to the complexity of mixture of ingredients is typically strictly avoided.

For example, in a preferred embodiment, the method according to the invention involves the adjustment of the pH value of the virgin liquid nutrient in step (ii-a), and wherein, preferably, the pH value is adjusted to any pH value selected from the group consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, or 9.9; and more preferably selected from the group consisting of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.0, 7.1, 7.2, 7.3, 7.4, 7.5, and even more preferably selected from the group consisting of pH 5.0 to 7.5, pH 3.0 to pH 6.0, pH 4.0 to 7.0, pH 3.5 to 6.5, pH 4.0 to 6.5, and pH 4.5 to 6.5.

Preferably, the method according to the invention involves the adjustment of the pH value of the virgin liquid nutrient in step (ii-a), wherein the virgin liquid nutrient is liquid milk, i.e. UHT milk or yogurt, and wherein, preferably, the adjustment of the pH value to any pH value selected from the group consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, or 9.9; and more preferably selected from the group consisting of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, and even more preferably selected from the group consisting of pH 4.0 to 6.5, pH 4.5 to 6.5, pH 5 to 6.5, pH 3.5 to 6.5, pH 4.0 to 6.5, and pH 4.5 to 6.5.

Preferably, the method according to the invention involves the adjustment of the pH value of the virgin liquid nutrient in step (ii-a), wherein the virgin liquid nutrient is a mixture of liquid milk and a food preparation, or of liquid milk and extracted fruit juice, and wherein, preferably, the adjustment of the pH value to any pH value selected from the group consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, or 9.9; and more preferably selected from the group consisting of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, and even more preferably selected from the group consisting of pH 3.5 to 6.5, pH 4.0 to 6.5, pH 4.5 to 6.5, pH 5 to 6.5, pH 3.5 to 6.5, pH 4.0 to 6.5, and pH 4.5 to 6.5.

Preferably, the method according to the invention involves no adjustment of the pH value of the virgin liquid nutrient in step (ii-a).

While it is theoretically contemplated that the one or more altered carbohydrates in the processed liquid nutrient may be further supplemented from an external source, preferably the total quantity of altered carbohydrates that is contained in the processed liquid nutrient originates either from quantities already originally contained in the virgin liquid nutrient or from enzymatic conversion, but not from an external source.

The virgin liquid nutrient comprises at least one initial carbohydrate, preferably selected from the group consisting of sucrose, inulin, lactose, glucose, galactose, starch, maltose, and fructose. Typically, said at least one initial carbohydrate originates from a natural source which can be a plant or an animal, e.g. vegetable, fruit, grain, pulse, nut, and milk.

The virgin liquid nutrient does not necessarily need to be a crude natural product. It is also contemplated that the virgin liquid nutrient has undergone certain process steps such as fractionation, filtration, clarification, homogenization, pasteurization, purification and the like. Examples for liquid nutrients having undergone certain process steps are dairy products (whey, cheese, curd, yoghurt, or other fermented milk derivatives), or derivatives from freshly pressed fruit or vegetable juice, like purees, concentrates, dehydrated juices, juice blends, or nectars. Nonetheless, the virgin liquid nutrient is preferably not an isolated single chemical entity or a defined composition comprising such an isolated single chemical entity, but a rather complex mixture of various ingredients. Typically, the virgin liquid nutrient contains various macronutrients (carbohydrates and/or proteins, lipids) and/or micronutrients (dietary minerals and/or vitamins) as additional ingredients.

Preferably, such various additional ingredients are not subject to the enzymatic conversion and thus, the individual quantities contained in the virgin liquid nutrient essentially correspond to the individual quantities in the processed liquid nutrient.

The virgin liquid nutrient preferably contains at least one, more preferably at least two, or at least three, or at least four, still more preferably at least five, or at least six, or at least seven, yet more preferably at least eight, or at least nine, or at least ten, even more preferably at least eleven, or at least twelve, or at least 13, most preferably at least 14, or at least 15, or at least 16, and in particular at least 17, or at least 18, or at least 19 additional ingredients independently of one another selected from the group consisting of the biomolecule species
lipids (i.e. lipoids and lipids, e.g. cholesterol, sitosterols, phospholipids, triglycerides, diglycerides, monoglycerides, fats, saturated fats, unsaturated fats, polyunsaturated fats, glycolipids, glycoshingolipids and gangliosides, and the like),
proteins (i.e. organic compounds consisting of amino acids joined by peptide bonds, which optionally may further be glycosylated),
vitamins (i.e. organic compounds that an organism needs in small quantities for the proper functioning of its metabolism),
metabolites (e.g. organic acids like citric acid, lactic acid, oxalic acid, acetic acid, and the like),
colloids and colloidal particles,
phytochemicals (e.g. carotenoids and polyphenols such as phenolic acids, flavonoids or stilbenes/lignans), and
polysaccharides other than starch or fibers (i.e. dietary fibers or roughage that are typically considered as carbohydrate polymers with more than 10 monomeric units, which are not hydrolyzed by digestive enzymes in the small intestine of humans; e.g. ß-glucans such as cellulose and chitin; hemicelluloses; lignin; xanthan gum; resistant starch; inulin; polyuronides such as pectin and alginic acids; raffinose, xylose, polydextrose, or lactulose).

Preferably, said virgin liquid nutrient is a complex mixture comprising at least 10 different substances including said one or more initial carbohydrates and including said one or more additional ingredients. For example, according to Bundeslebensmittelschüissel (www.blsdb.de), a representative apple juice typically contains in 100 ml inter alia the following ingredients: 1.9 g glucose, 5.3 g fructose, 2.4 g sucrose, 0.3 g protein, 0.3 g fat, 7.4 mg vitamin C, 0.1 mg pantothenic acid, 126 mg potassium and 0.5 mg iron.

A virgin liquid nutrient may comprise different chemical entities from one biomolecule species of additional ingredients. Milk and yogurt contain, for example, several proteins of the casein family, several proteins from the serum (or whey) family and enzymes (lipases, catalases, peroxidases, phosphatases), several vitamins, and over 400 individual fatty acids in form of mono-, di-, or tri-acyl glycerides in different percentages. Table 5 exemplifies possible additional ingredients and their content range of the virgin liquid nutrients according to this invention.

Preferably, the total content of said one or more additional ingredients is at least 0.1 wt.-%, preferably at least 0.5 wt.-%, or at least 1.0 wt.-%, or at least 2.0 wt.-%, more preferably at least 3.0 wt.-%, or at least 4.0 wt.-%, or at least 5.0 wt.-%, still more preferably at least 6.0 wt.-%, or at least 7.0 wt.-%, or at least 8.0 wt.-%, yet more preferably at least 9.0 wt.-% or at least 10 wt.-%, or at least 11 wt.-%, even more preferably at least 12 wt.-%, or at least 13 wt.-%, or at least 14 wt.-%, most preferably at least 15 wt.-%, or at least 16 wt.-%, or at least 17 wt.-%, and in particular at least 18 wt.-%, or at least 19 wt.-%, or at least 20 wt.-%, or at least 21 wt.-%, or at least 22 wt.-%, or at least 23 wt.-%, or at least 24 wt.-%, or at least 25 wt.-%, or at least 26 wt.-%, or at least 27 wt.-%, or at least 28 wt.-%, or at least 29 wt.-%, or at least 30 wt.-%, or at least 31 wt.-%, even more preferably at least 32 wt.-%, or at least 33 wt.-%, or at least 34 wt.-%, or at least 35 wt.-%, or at least 36 wt.-%, or at least 37 wt.-%, or at least 38 wt.-%, or at least 39 wt.-%, or at least 40 wt.-%, or at least 41 wt.-%, or at least 42 wt.-%, or at least 43 wt.-%, or at least 44 wt.-%, or at least 45 wt.-%, or at least 46 wt.-%, or at least 47 wt.-%, or at least 48 wt.-%, or at least 49 wt.-%, or at least 50 wt.-%, or at least 51 wt.-%, or at least 52 wt.-%, or at least 53 wt.-%, or at least 54 wt.-%, or at least 55 wt.-%, or at least 56 wt.-%, or at least 57 wt.-%, or at least 58 wt.-%, or at least 59 wt.-%, or at least 60 wt.-%, or at least 61 wt.-%, or at least 62 wt.-%, or at least 63 wt.-%, or at least 64 wt.-%, or at least 65 wt.-%, or at least 66 wt.-%, or at least 67 wt.-%, or at least 68 wt.-%, or at least 69 wt.-%, or at least 70 wt.-%, or at least 71 wt.-%, or at least 72 wt.-%, or at least 73 wt.-%, or at least 74 wt.-%, or at least 75 wt.-%, or at least 76 wt.-%, or at least 77 wt.-%, or at least 78 wt.-%, or at least 79 wt.-%, or at least 80 wt.-%, or at least 81 wt.-%, or at least 82 wt.-%, or at least 83 wt.-%, or at least 84 wt.-%, or at least 85 wt.-%, or at least 86 wt.-%, or at least 87 wt.-%, or at least 88 wt.-%, or at least 89 wt.-%, or at least 90 wt.-%, or at least 91 wt.-%, or at least 92 wt.-%, or at least 93 wt.-%, or at least 94 wt.-%, or at least 95 wt.-%, or at least 96 wt.-%, or at least 97 wt.-%, or at least 98 wt.-%, or at least 99 wt.-% relative to the total weight of said virgin liquid nutrient, preferably relative to the dry weight of the virgin liquid nutrient.

Preferably, said one or more initial carbohydrates originate from a natural source; and said one or more additional ingredients originate from the same natural source as said one or more initial carbohydrates.

Thus, the virgin liquid nutrient according to the invention is distinguished from liquid compositions conventionally employed as well-defined starting materials for enzymatic conversions that are conventionally prepared in laboratories from commercially available products containing single chemical substances in highly pure form. On the contrary, the virgin liquid nutrient according to the invention is typically characterized in that it comprises a complex mixture of various ingredients. Typically, the specific composition of the virgin liquid nutrient according to the invention is even unknown, i.e. the specific amount of each and every ingredient has not been determined. Representative examples of virgin liquid nutrients according to the invention include but are not limited to milk, fruit juices, vegetable juices, and the like. Preferably, the virgin liquid nutrient is selected from the group consisting of liquid milk, extracted fruit juice, and food preparations.

Mixtures of different virgin liquid nutrients are also contemplated. Preferred subgroups of virgin liquid nutrients according to the invention are (a) liquid milk, (b) extracted fruit juice, and (c) food preparations.

For the purpose of the specification, food preparations include but are not limited to carbohydrate compositions such as honey or syrup, wherein syrup is preferably derived from starch, grain, rice or vegetable processing (e.g. high fructose corn syrup, rice syrup, grain syrup, barley syrup, or the like).

Therefore, it is also within the scope of the invention that the virgin liquid nutrient, which contains the one or more initial carbohydrates as starting materials for enzymatic conversions, is
a mixture of one or more virgin liquid nutrients from the same subgroup (e.g. a mixture of two different liquid milks, or a mixture of two different extracted fruit juices, or a mixture of two different food preparations) or
a mixture of one or more virgin liquid nutrients from one subgroup with one or more virgin liquid nutrients from another subgroup,
   in either case resulting in a combined virgin liquid nutrient.

Said combined liquid virgin nutrient is typically characterized in that the overall composition is a complex mixture of various ingredients. Representative examples of such combined virgin liquid nutrients according to the invention include but are not limited to mixtures of milk with fruit purees, milk with fruit concentrates, yogurt with fruit purees, yogurt with fruit concentrates, milk with carbohydrate compositions, fruit purees with carbohydrate compositions, and fruit concentrates with carbohydrate compositions; wherein in each case carbohydrate compositions may include but are not limited to honey or syrup, wherein syrup is preferably derived from starch, grain, rice or vegetable processing (e.g. high fructose corn syrup, rice syrup, grain syrup, barley syrup, or the like).

For example, in a preferred embodiment of the method according to the invention, the virgin liquid nutrient comprises a mixture of liquid milk and extracted fruit juice, wherein said mixture contains fructose as initial carbohydrate, and wherein step (iv) involves the enzymatic conversion of at least a portion of the fructose into D-allulose. Preferably, the liquid milk is yogurt, diluted yogurt, milk, or UHT milk.

Likewise, in a preferred embodiment of the method according to the invention, the virgin liquid nutrient comprises a mixture of liquid milk and honey, wherein said mixture containing fructose as initial carbohydrate, and wherein step (iv) involves the enzymatic conversion of at least a portion of the fructose into D-allulose. Preferably, the liquid milk is yogurt, diluted yogurt, milk, or UHT milk.

Preferably, the method according to the invention is for preparing an edible processed liquid nutrient by enzymatic in-situ conversion of a virgin liquid nutrient, the method comprising the steps of
(i) providing a virgin liquid nutrient which comprises
   one or more initial carbohydrates selected from the group consisting of sucrose, inulin, lactose, glucose, galactose, starch, maltose, and fructose; and
   preferably, one or more additional ingredients selected from the group consisting of lipids, proteins, vitamins, metabolites, colloids or colloidal particles, phytochemicals, fibers, and polysaccharides other than starch;
(ii) optionally, adjusting (ii-a) pH value and/or (ii-b) temperature of the virgin liquid nutrient;
(iii) optionally, supplementing (iii-a) inorganic phosphate and/or (iii-b) enzyme cofactor and/or (iii-c) one or more initial carbohydrates to the virgin liquid nutrient; and
(iv) treating the virgin liquid nutrient with one or more enzymes, thereby converting at least a portion of the one or more initial carbohydrates into one or more altered carbohydrates selected from the group consisting of kojibiose, nigerose, trehalose, cellobiose, alpha-D-fructofuranose beta-D-fructofuranose 1,2′:2,3′-dianhydride (DFA III), D-allulose, D-tagatose, isomaltulose, IMOs, GlucOS, isomaltose, and D-mannose; thus obtaining the processed liquid nutrient.

The method according to the invention provides an edible processed liquid nutrient by enzymatic in-situ conversion of at least a portion of the one or more initial carbohydrates that are contained as starting materials in the virgin liquid nutrient and that are enzymatically converted in-situ into one or more altered carbohydrates selected from the group consisting of kojibiose, nigerose, trehalose, cellobiose, alpha-D-fructofuranose beta-D-fructofuranose 1,2′:2,3′-dianhydride (DFA III), D-allulose, D-tagatose, isomaltulose, isomaltose, isomalto-oligosaccharides (IMO), gluco-oligosaccharides (GlucOS), and D-mannose; thereby obtaining the processed liquid nutrient. Thus, said one or more altered carbohydrates are not added from an external source, neither to the virgin liquid nutrient nor to the edible processed liquid nutrient, but prepared in-situ by enzymatic conversion of one or more initial carbohydrates into one or more altered carbohydrates.

The method according to the invention is directed to the enzymatic processing of a virgin liquid nutrient comprising one or more initial carbohydrates into a processed liquid nutrient. Thus, in other words, the method according to the invention is directed to the preparation of a processed liquid nutrient (product) from a virgin liquid nutrient (starting material), wherein the method involves enzymatically catalyzed conversion of at least one initial carbohydrate that is contained in the virgin liquid nutrient into one or more altered carbohydrates that are contained in the processed liquid nutrient. Preferably, the processed liquid nutrient is edible, more preferably devoted for end use by a consumer, e.g. for consumption. The processed liquid nutrient may or may not contain the enzyme or the enzymes that catalyzed the conversion. When the processed liquid nutrient contains the enzyme or the enzymes that catalyzed the conversion, the enzyme or the enzymes independently of one another may be present in active or deactivated state.

Preferably, the method according to the invention involves enzymatic in-situ conversion of a virgin liquid nutrient, in particular enzymatic in-situ conversion of one or more initial carbohydrates that are contained in said virgin liquid nutrient. For the purpose of the specification, in-situ conversion means that the enzymatic conversion of the one or more initial carbohydrates takes place within the liquid nutrient. The initial carbohydrates are therefore not isolated from the virgin liquid nutrient but enzymatically converted into the one or more altered carbohydrates in the presence of all other constituents of the liquid nutrient.

The processed liquid nutrient is typically edible, i.e. contains no substances that are harmful for the human body. Thus, in case that the one or more enzymes that are employed in the enzymatic conversion should not be physiologically acceptable for some reasons, they are typically removed from the processed liquid nutrient after conversion or inactivated by suitable measures that are known to the skilled person.

Preferably, the textural sensation conferred by all initial carbohydrates and altered carbohydrates contained in the processed liquid nutrient and the textural sensation conferred by all initial carbohydrates contained in the virgin liquid nutrient is expressed as
the viscosity or viscoelasticity conferred by all the carbohydrates, wherein the viscosity or viscoelasticity conferred by all initial carbohydrates and altered carbohydrates of the processed liquid nutrient in comparison to the viscosity or viscoelasticity conferred by all initial carbohydrates contained in the virgin liquid nutrient is connatural; and/or
the crystallinity conferred by all the carbohydrates, wherein the crystallinity conferred by all initial carbohydrates and altered carbohydrates of the processed liquid nutrient in comparison to the crystallinity conferred by all initial carbohydrates contained in the virgin liquid nutrient is connatural.

Methods for the measurement of viscosity, viscoelasticity and crystallinity of carbohydrate-containing liquids and preparation are known in the art. The viscosity is usually measured as dynamic viscosity (Unit: 1 PA s) or alternatively as kinematic viscosity.

Preferably, the viscosity or viscoelasticity conferred by all initial carbohydrates and altered carbohydrates contained in the processed liquid nutrient and the viscosity or viscoelasticity conferred by all initial carbohydrates differs by from 0 to 10%, preferably from 0 to 5%, more preferably from 0 to 2.5%, and most preferably from 0 to 1%.

Preferably, the crystallinity conferred by all initial carbohydrates and altered carbohydrates contained in the processed liquid nutrient and the crystallinity conferred by all initial carbohydrates differs by from 0 to 10%, preferably from 0 to 5%, more preferably from 0 to 2.5%, and most preferably from 0 to 1%.

For the purpose of this invention, “glycemic index” describes a number associated with the carbohydrates in a particular type of food that indicates the effect of these carbohydrates on a human's blood glucose (also called blood sugar) level. The glycemic index represents the rise in a human's blood sugar level two hours after consumption of the food. A value of 100 represents the standard, an equivalent amount of pure glucose. For each foodstuff, a specific glycemic index, which is also called “relative glycemic response”, or RGR, can be calculated based thereon. The glycemic index effect of food depends on a number of factors, such as the type of carbohydrate. The glycemic index is useful for understanding how the human body breaks down carbohydrates and takes into account only the available carbohydrate (total carbohydrate minus fiber) in a food.

For the purpose of this invention, “calorie count” describes the physiological energy content of initial and altered carbohydrates if metabolized by the human body, corresponding to the metabolizable energy (ME). By definition, one calorie (kcal) is the energy needed to raise the temperature of 1 kg water by 1° C. Alternatively the energy content of food is expressed in kilojoules (kJ). One kcal equals to 4.184 kJ. Originally, the number of kcals in a given food or its constituting components (as for example stipulated in The Nutrition Labeling and Education Act (NLEA) of 1990) is directly measured by its burning in a bomb calorimeter while the resulting increase of temperature in surrounding water is measured. However, major shortcoming of this method is that the physical rather than the physiology energy content is measured. For instance, food may contain carbohydrate fibers that are not digested and utilized by the body. In that case the fiber component is usually subtracted from the total carbohydrate before calculating the calories. However, there are functional carbohydrates that are either partly digested and then utilized by the human body, or that are digested by the human microbiome. The microbiome itself produces metabolites that can be digested and utilized by the body as well as autolysis of the microbiome releases compounds of nutritional value. For all these reasons the true physiological energy count of a functional carbohydrate has to be determined as energy conversion factor. Energy conversion factors for functional carbohydrates for the purpose of nutrition labelling have been set based on the concept of metabolizable energy (ME). Depending on the available data an energy conversion factor for a nutrient can be defined. Ideally, tangible data regarding the absorption, distribution, metabolism and excretion of the respective nutrient are available to calculate an accurate energy conversion factor for the respective functional carbohydrate-based on the concept of ME.

The calorie count values and glycemic indices of monosaccharide and disaccharide carbohydrates of the invention are summarized in Table 1.

TABLE 1
glycemic indices and calorie count values of carbohydrates:

| | Calorie count | Glycemic index |
|---|---|---|
| Carbohydrate | (ME) [kcal/g] | (relative glycemic response) |
| lactose | 4.0 | 46% |
| sucrose | 4.0 | 68% |
| glucose | 3.9 | 100% |
| galactose | 3.9 | 50% |
| fructose | 3.9 | 19% |
| D-allulose | 0.2 | 0% |
| isomaltulose | 4.0 | 32% |
| D-tagatose | 1.5 | 3% |
| D-mannose | 3.9 | 25% |
| trehalose | 4.0 | 72% |
| cellobiose | 2.0 | 5% |
| kojibiose | 2.0 | 5% |
| nigerose | 2.0 | 5% |
| DFA III | 0.3 | 0% |
| IMO | 2.0 | 35% |
| GlucOS | 2.0 | 35% |

For the purpose of this invention, “textural sensation” describes a certain aspect which is perceived by the tongue as a physical feeling during the intake of food, and which is different from sweet, sour, bitter, and/or salty sensation, and which creates a certain mouthfeel. It is therefore a subcategory of the organoleptic properties of food that individual experiences via all senses, including sight, touch, taste, and smell, which altogether play pivotal roles in product acceptability. The textural sensation of food is a multidimensional sensory property that is influenced by the food's structure, rheology and surface properties. Textural sensation of food, comprising solid, semi-solid or liquid foods or beverages, is experienced by an individual at the point at which food enters the mouth. It is therefore, for example, but not limited to, perceived as the initial thickness of a liquid food and for example, but not limited to, perceived as sandiness of a solid food. One element of textural sensation of food, the sandiness, or crystallinity, is greatly impacted by the presence of for example crystalline sugars. Another element of textural sensation of relevance relating to the thickness of liquid semi-solid food is characterized by its rheological properties, which can be assessed as viscosity and viscoelasticity. For instance, the thickness of food gets increased if the content of oligo- or polysaccharides is increased. In case of milk the underlying mechanism of textural perception is hypothesized to arise from mechanoreceptor stimulation by the viscosity of this oil-in-water emulsion and from certain characteristics of the lipid/fat globules. The viscosity is increased if oligosaccharides like GOS are present. Additionally, oligosaccharides may bind water to act as hydrocolloids and then are impacting the rheology to a greater extent than just by their molecular size. For the purpose of the invention, the crystallinity and the viscosity or viscoelasticity are the most relevant elements for judging the textural sensation of a liquid nutrient.

For the purpose of this invention “sweetness” describes a basic taste most commonly perceived when eating foods rich in sugars. Sweet tastes are regarded as a pleasurable experience. Individual carbohydrates differ greatly in their sweetness profile which is typically assessed by a qualified test panel under consideration of multiple sub-parameters like for example, but not limited to, on-set, off-set, after-taste and palatability. For the purpose of this invention sweetness is assessed via a bioassay using Drosophila melanogaster (Gordesky-Gold, B. et al., Chem Senses, 2008 March; 33(3): 301-309), which is known to expose a preference to sweet test substances in a suitable experimental setup. It is within the scope of this invention, that the sweetness of a processed liquid nutrient is detected by this bioassay or other suitable assay formats.

The term “virgin liquid nutrient” in the meaning of this invention describes the virgin liquid nutrient (starting material) that is subjected to the method according to the invention and provided in step (i) above. It is understood, that such virgin liquid nutrient according to the invention may be a liquid preparation from raw materials, like from raw milk, fruits or vegetables, primary fruit or vegetables extracts or others, which has undergone extensive processing steps prior to be subjected to the invention as an “virgin liquid nutrient”. It is also within the meaning of this invention, that the virgin liquid nutrient in addition to the one or more initial carbohydrates preferably contains at least one or more additional ingredients independently of one another selected from the group consisting of the biomolecule species lipids, proteins, vitamins, metabolites (e.g. organic acids like citric, lactic, oxalic, acetic acids), colloids or colloidal particles, phytochemicals (e.g. carotenoids and polyphenols such as phenolic acids, flavonoids or stilbenes/lignans), fibers, and polysaccharides other than starch. It is also within the scope of the invention, that the virgin liquid nutrient is a mixture of one or more virgin liquid nutrients, or a mixture of one or more virgin liquid nutrients with one or more well-defined supplemented starting materials, resulting in a “combined” starting material of virgin liquid nutrient in the meaning of the invention.

The term “processed liquid nutrient” in the meaning of this invention describes the processed liquid nutrient (product) that is obtained by the method according to the invention in step (iv) above. It is understood, that such processed liquid nutrient according to the invention may undergo further subsequent extensive processing steps prior to be used by the end user, e.g. consumed.

The term “connatural” in the meaning of this invention describes a qualitative and/or quantitative term to specify the differences of a processed liquid nutrient from a virgin liquid nutrient in terms of textural sensation, viscoelasticity, viscosity or sweetness, wherein the term shall mean that the corresponding functional characteristic of the processed liquid nutrient is identical, and/or at least close to identical, and or at least closely comparable to the virgin liquid nutrient. Specifically, in respect to the physicochemical properties viscosity, viscoelasticity, and crystallinity, the term “connatural” shall mean that the specific measured value for one certain property of the processed liquid nutrient shall deviate not more than from 0 to 10%, preferably not more than from 0 to 5%, more preferably not more than from 0 to 2.5%, and most preferably not more than from 0 to 1%.

Step (i), optional step (ii), optional step (iii) and step (iv) of the method according to the invention are typically performed in numerical order. A skilled person recognizes that it does not matter whether optional step (ii) is performed before or after step (iii) or simultaneously with step (iii). It is contemplated that any of these steps may be performed simultaneously or partially simultaneously.

Step (i) of the method according to the invention involves the provision of a virgin liquid nutrient which comprises at least one initial carbohydrate. It is contemplated that the virgin liquid nutrient may already comprise the at least one initial carbohydrate, e.g. by nature. Alternatively, the at least one initial carbohydrate may have been added to the virgin liquid nutrient or the content of the at least one initial carbohydrate may have been enriched by addition thereof.

Step (ii) of the method according to the invention is optional and may involve adjusting

(ii-a) pH value and/or
(ii-b) temperature of the virgin liquid nutrient.

Step (iii) of the method according to the invention is also optional and may involve supplementing

(iii-a) inorganic phosphate and/or
(iii-b) cofactors and/or
(iii-c) one or more initial carbohydrates.

Step (iv) of the method according to the invention is involves the treatment of the virgin liquid nutrient with one or more enzymes, thereby converting at least a portion of the at least one initial carbohydrate into one or more altered carbohydrates and thus containing the processed liquid nutrient. It is contemplated that the initial carbohydrate may be converted into one or more altered carbohydrates in a single reaction step or in a sequence of two or more reaction steps. Thus, the initial carbohydrate may be converted into one or more first intermediate carbohydrates in a first reaction step catalyzed by a first enzyme, and one or more of said first intermediate carbohydrates may be converted into one or more second intermediate carbohydrates in a second reaction step catalyzed by a second enzyme, and one or more of said second intermediate carbohydrates may be converted into the one or more altered carbohydrates in a third reaction step catalyzed by a third enzyme.

Preferably, the at least one altered carbohydrate is selected from the group consisting of monosaccharides and/or disaccharides.

In a preferred embodiment, the at least one altered carbohydrate comprise or essentially consist of disaccharides. In a preferred embodiment, the disaccharide is reducing. In another preferred embodiment, the disaccharide is non-reducing. Preferably, the disaccharide is composed of units independently selected from the group consisting of glucose, galactose, fructose, rhamnose, and mannose. Preferably, the linkage of the two units is selected from the group consisting of α(1→2), α(1→3), α(1→4), α(1→6), β(1→2), β(1→3), β(1→4), and β(1→6).

In a preferred embodiment, the disaccharide is a common disaccharide. Preferred common disaccharides include but are not limited to sucrose, lactulose, lactose, maltose, isomaltose, trehalose, and cellobiose.

In another preferred embodiment, the disaccharide is a rare disaccharide. Preferred rare disaccharides include but are not limited to kojibiose, nigerose, isomaltulose, isomaltose, trehalose, and laminaribiose.

In another preferred embodiment, the at least one altered carbohydrate comprise or essentially consist of monosaccharides.

Preferably, the at least one altered carbohydrate is a natural carbohydrate. Natural carbohydrate in the meaning of this invention means that such carbohydrates are occurring in and/or synthesized by nature.

Preferably, the at least one initial carbohydrate is selected from the group consisting of monosaccharides, disaccharides, oligosaccharides and/or polysaccharides.

Preferably, the virgin liquid nutrient is selected from the group consisting of
liquid milk; and/or
extracted fruit juice; and/or
food preparations.

Table 2 shows an exemplary excerpt of functional carbohydrates (altered carbohydrates) that can be enzymatically produced out of lactose, glucose and/or galactose that are naturally occurring carbohydrates (initial carbohydrates) in milk-based virgin liquid nutrients:

Liquid milk and products derived thereof

| | | Sweetness, | | |
|---|---|---|---|---|
| Naturally | Calorie | relative to | | |
| contained | Count | sucrose | | |
| carbohydrates | [kcal/g] | [ ] | (Main) Benefit | Drawback |
| Lactose | <4 | 0.16 | delivers the essential | high calorie count/ |
| Glucose | 3.9 | 0.7 | — | promotes tooth decay |
| Galactose | 3.9 | 0.6 | essential for | |
| | | | development | |
| | | | (“brain sugar”) | |

| Functional | | Sweetness, | | Naturally contained | | | |
|---|---|---|---|---|---|---|---|
| Ingredient that | Calorie | relative to | | Carbohydrate used | | | |
| can be formed | Count | sucrose | | as Substrate for | | E.C. Number of | Equlibrium/ |
| in-situ | [kcal/g] | [ ] | (Main) Benefits | respective Ingredient | Process/Enzymes | Enzyme | Conversion |
| D-Allulose | 0.2 | 0.9 | very low-caloric | Lactose and Glucose | Isomerization of | D-psicose 3- | D-fructose:D- |
| | | | sweetener, low- | | fructose; additional | epimerase | psicose |
| | | | glycemic | | glucose by hydrolysis | EC 5.1.3.30 | 70:30 |
| | | | | | of sucrose | | |
| D-Tagatose | 1.5 | 0.7 | low-caloric | Lactose and Galactose | Isomerization of | L-Arabinose- | D-galactose:D- |
| | | | sweetener, low- | | galactose; additional | Isomerase EC | tagatose |
| | | | glycemic | | galactose by hydrolysis | 5.3.1.4 | 60:40 |
| | | | | | of lactose | | |
| D-Mannose | 3.9 | 0.3 | prevents bladder | Lactose and Glucose | Isomerization of glucose | Glucose | D-glucose l D- |
| | | | infection, | | to fructoe followed by | isomerase EC | fructose |
| | | | prebiotic | | an isomerization of | 5.3.1.5 and | 45:55 |
| | | | | | fructose to glucose | Mannose isomerase | D-fructose:D- |
| | | | | | (additional glucose by | EC 5.3.1.7 | mannose |
| | | | | | hydrolysis of lactose | | 70:30 |
| GOS | 1.7 | 0.3 | prebiotic, fibre | Lactose | Elongation of lactose | β-galactosidase | >90% |
| | | | | | by galactose using | EC 3.2.1.23 | |
| | | | | | the enzymes β- | | |
| | | | | | galactosidase | | |

Table 3 shows an exemplary excerpt table of functional carbohydrates (altered carbohydrates) that can be enzymatically produced out of sucrose, glucose, starch, maltose, and/or fructose and/or inulin and other polysaccharides, that are naturally occurring carbohydrates (initial carbohydrates) in fruit-based virgin liquid nutrients:

Liquid extracts obtained from fruits and products derived thereof

| Naturally | Calorie | | | |
|---|---|---|---|---|
| contained | Count | ss, | | |
| carbohydrates | [kcal/g] | relative to | (Main) Benefit | Drawback |
| Sucrose | 4 | 1 | provides sweetness | high calorie count/ |
| | | | and texture | promotes tooth decay |
| Fructose | 3.9 | 1.1 | provides sweetness | |
| Glucose | 3.9 | 0.7 | — | |
| Inulin | 1.5 | 0.3 | prebiotic fibre | — |
| Pectin | 3 | — | stabilizer, fibre | may cause turbidity, |
| | | | | may hamper |
| Maltose | 4 | 0.5 | sweetness, texture | high calorie count/ |
| Starch | 4 | — | energy, texture | promotes tooth decay |

| Functional | | ss, | | | |
|---|---|---|---|---|---|
| Ingredient that | Calorie | relative to | | Carbohydrate used | |
| can be formed | Count | sucrose | | as Substrate for | |
| in-situ | [kcal/g] | [ | (Main) Benefits | respective Ingredient | Process/Enzymes |
| Allulose | 0.2 | 0.9 | very low-caloric | Sucrose and Fructose | Isomerization of fructose by an |
| | | | sweetener, low- | | epimerase (additional glucose |
| | | | glycemic | | by hydrolysis of sucrose) |
| Mannose | 3.9 | 0.6 | prevents bladder | Sucrose and Fructose | Isomerization of fructose by an |
| | | | infection, prebiotic | | epimerase (additional fructose |
| | | | | | by hydrolysis of sucrose and/or |
| | | | | | isomerization of glucose) |
| Kojibiose | 2 | not yet | prebiotic | Sucrose | Transfers the glucose of sucrose |
| | | known | | | to another glucose molecule |
| Isomaltulose | 4 | 0.5 | low-glycemic | Sucrose | Isomerization of sucrose |
| | | | sweetener | | |
| Trehalose | 4 | 0.5 | stabilizing sugar, | Sucrose | Sucrose phosphorylase for |
| | | | low-glycemic | | phosphorylsis of sucrose, glucose-1- |
| | | | sweetener | | phosphate gets transfered to |
| | | | | | glucose by trehalose phosphorylase |
| Cellobiose | 2 | 0.3 | prebiotic, taste | Sucrose | Sucrose phosphorylase for |
| | | | modifier | | phosphorylsis of sucrose, glucose-1- |
| | | | | | phosphate gets transfered to |
| | | | | | glucose by cellobiose |
| | | | | | phosphorylase |
| Difructose | 0.3 | 0.5 | very low-caloric | Inulin | Degradation of Inulin |
| anhydride | | | sweetener, low- | | |
| | | | glycemic | | |
| FOS | 1.5 | 0.3 | prebiotic, fibre | Sucrose | Elongation of sucrose by fructose |
| Arabinose | 3 | 0.5 | natural sucrase | Pectin, arabinan, | Hydrolysis of substrates by |
| | | | inihibitor | arabinoxylan or | pectinases |
| | | | | arabinogalactan | |
| IMO | 2 | 0.5 | prebiotic, fibre | Sucrose, Glucose, | Hydrolysis of strach; |
| | | | | Starch | transglycosidation |
| GlucOS | 2 | 0.5 | prebiotic, fibre | Sucrose, Glucose, | Transglycosidation |
| | | | | Maltose | |

| Functional | | |
|---|---|---|
| Ingredient that | | |
| can be formed | | Equlibrium/ |
| in-situ | E.C. Number of Enzyme | Conversion |
| Allulose | D-psicose 3-epimerase EC 5.1.3.30 | D-fructose:D-psicose |
| | | 70:30 |
| Mannose | Mannose isomerase EC 5.3.1.7 | D-fructose:D-mannose |
| | | 70:30 |
| Kojibiose | Sucrose phosphorylase EC 2.4.1.7 | >90% |
| Isomaltulose | Isomaltulose synthase EC 5.4.99.11 | >90% |
| Trehalose | Sucrose phosphorylase EC 2.4.1.7 and | >90% |
| | Trehalose phosphorylase EC 2.4.1.64 | |
| Cellobiose | Sucrose phosphorylase EC 2.4.1.7 and | >90% |
| | Cellobiose phosphorylase EC 2.4.1.20 | |
| Difructose | Inulin-Fructotransferases | >90% |
| anhydride | | |
| FOS | Fructosyltransferase EC 2.4.1.9 and | >90% |
| | β-fructofuranoidase EC 3.2.1.26) | |
| Arabinose | various | — |
| IMO | α-amylase (EC 3.2.1.1), | |
| | β-amylase (EC 3.2.1.2), | |
| | pullulanase (EC 3.2.1.41), | |
| | dextran sucrase (EC 2.4.1.5), | |
| | alpha-transglucosidase (EC 2.4.1.24) | |
| GlucOS | dextran sucrase (EC 2.4.1.5) | |

Table 4 summarizes the typical content of initial carbohydrates in fruits:

| | grams sugar per 100 grams | | | | |
|---|---|---|---|---|---|
| | Total sugar | Glucose | Fructose | Sucrose | pH |
| Apples | 13.3 | 2.30 | 7.60 | 3.30 | 3.3-3.9 |
| Apricot | 9.3 | 1.60 | 0.70 | 5.20 | 3.3-4.8 |
| Banana | 15.6 | 4.20 | 2.70 | 6.50 | 4.5-5.2 |
| Blackberries | 8.1 | 1.30 | 4.10 | 0.40 | 3.9-4.5 |
| Blueberries | 7.3 | 3.50 | 3.60 | 0.20 | 3.1-3.4 |
| Cherries, sweet | 14.6 | 8.10 | 6.20 | 0.20 | 3.2-4.5 |
| Cherries, sour | 8.1 | 4.20 | 3.30 | 0.50 | 3.3-3.5 |
| Grapefruit | 6.2 | 1.30 | 1.20 | 3.40 | 3.0-3.7 |
| Grapes | 18.4 | 8.10 | 8.30 | 2.00 | 3.4-4.5 |
| Lemon | 2.5 | 1.00 | 0.80 | 0.60 | 2.2-2.4 |
| Mango | 14.8 | 0.70 | 2.90 | 9.90 | 5.8-6.0 |
| Nectarine | 8.5 | 1.20 | 1.00 | 6.20 | 3.9-4.2 |
| Orange | 9.2 | 2.20 | 2.50 | 4.20 | 3.0-4.0 |
| Papaya | 5.9 | 1.40 | 2.70 | 1.80 | 5.2-6.0 |
| Peach | 8.7 | 1.20 | 1.30 | 5.60 | 3.4-4.1 |
| Pear | 10.5 | 1.90 | 6.40 | 1.80 | 3.6-4.0 |
| Pineapple | 11.9 | 2.90 | 2.10 | 3.10 | 3.2-4.0 |
| Raspberries | 9.5 | 3.50 | 3.20 | 2.80 | 3.2-3.6 |
| Strawberries | 5.8 | 2.20 | 2.50 | 1.00 | 3.0-3.9 |
| Tomato | 2.8 | 1.10 | 1.40 | 0.30 | 4.3-4.9 |
| Watermelon | 9 | 1.60 | 3.30 | 3.60 | 5.2-5.6 |

Table 5 summarizes the approximate or average non-carbohydrate contents of liquid milk, extracted fruit juices and the exemplary food preparations wheat roll, short bread, and whole meal rye bread:

| | wt.-% of non-carbohydrate content | | | | | | | | |
|---|---|---|---|---|---|---|---|---|---|
| | Liquid Milk | Yoghurt | Organge | Apple | Grape | Mango | Wheat | Short | Wholemeal |
| | (full fat) | (full fat) | Juice | Juice | Juice | Juice | Roll | Bread | Rye Bread |
| Water | 87 | 87 | 88 | 88 | 84 | | 36 | 20 | 40 |
| Fat | 4 | 3.6 | 0.2 | 0 | 0 | 0.1 | 1.2 | 23.6 | 1.3 |
| Protein | 3 | 4.1 | 0.7 | 0.1 | 0 | 0.3 | 8.9 | 6.7 | 5.5 |
| Fiber | 0 | 0 | 0.2 | 0.2 | 0.1 | 0.8 | 3.5 | | 10.3 |
| Minerals | <1 sodium, | <1 sodium, | <1 mainly | <1 mainly | <1 mainly | <1 mainly | 1 mainly | <1 | 1.2 sodium |
| | potassium, | zinc, | sodium, | potassium, | sodium, | sodium, | sodium | | chloride, |
| | calcium, | magnesium, | potassium, | magnesium, | potassium, | potassium, | chloride | | potassium, |
| | magnesium, | calcium, | iron, zinc, | cloride, | magnesium, | calcium | | | magnesium, |
| | phosphorous, | potassium | magniesium, | calcium, | calcium, | | | | calcium |
| | sufur, | | chloride, | iron | chloride, | | | | |
| | chloride, | | sufur, | | phosphorous, | | | | |
| | iron, zinc, | | calcium, | | sulfur | | | | |
| | copper, | | phosphorous | | | | | | |
| | manganese | | | | | | | | |
| Vitamins | mainly | mainly | mainly | mainly | mainly | mainly | | | |
| | vitamin A, | vitamin A, | vitamin C | vitamin C | vitamin C | Vitamin A, | | | |
| | Thiamin, | vitamin D. | | | | Vitamin C | | | |
| | Riboflavin, | vitamin E. | | | | | | | |
| | Niacin, | Thiamin, | | | | | | | |
| | pathoic acid, | Riboflavin, | | | | | | | |
| | pyridoxin, | Pyridoxin, | | | | | | | |
| | cobalaminvi- | cobalamin | | | | | | | |
| | tamin C | | | | | | | | |

For the purpose of this invention, “liquid milk” describes virgin liquid nutrients like milk and whey, both containing a whole lot of nutrients that are important for different biological processes in the human body and are therefore integral to human health. For example, vitamins and minerals like calcium are important for the development of strong bones and teeth as well as for muscle formation and cellular activity. Milk is a biphasic emulsion with fat/lipid particles (globules) dispersed in an aqueous (watery) environment, comprising casein micelles, proteins, lipids, carbohydrates and vitamins (biphasic milk emulsion). Dairy products are offered to the consumers in a plethora of variants, for example, but not limited to, as (pasteurized) yoghurt, cheese, whey-drink and whey-powder. The term “liquid milk” according to the invention also encloses such dairy products derived from milk and whey through certain partial processing, for example cheese, curd, yoghurt, or other fermented milk derivatives. For example, besides water, proteins, lipids, minerals and vitamins, bovine milk typically has a lactose content of 4.4 to 5.2 wt.-%. Also other carbohydrates like glucose, galactose, and bovine milk oligosaccharides are found in milk, but at very low concentrations (Walstra, P., Wouters, J., & Geurts, T. (2006). Dairy Science and Technology (2:a ed.). Taylor and Francis; Chapter 6 “Carbohydrates”; Corzo et. Al (2008) Handbook of Dairy Foods Analysis, Publisher). An overview of average non-carbohydrate components (additional ingredients) of liquid milk is given in Table 5.

For the purpose of this invention, “extracted fruit juice” describes virgin liquid nutrients that are made from the extraction or pressing out of the natural liquid contained in fruit or vegetables. Juice is commonly consumed as a beverage or used as an ingredient or flavoring in foods, such as candies, or other beverages, such as lemonades. Juice is prepared by mechanically squeezing or macerating fruit/vegetable flesh without the application of heat or solvents. Many commercial juices are filtered to remove fiber or pulp, however, high-pulp fresh orange juice is a popular beverage. Common methods for preservation and processing of fruit/vegetable juices include canning, pasteurization, concentrating, freezing, evaporation and spray drying. Although processing methods vary from juices, the general processing method of juices includes: juice extraction, straining, filtration and clarification. After the juice is filtered, it may be concentrated in evaporators, which reduce the size of juice by a factor of 5, making it easier to transport and increasing its expiration date. The juice is then later reconstituted, in which the concentrate is mixed with water and other factors to return any lost flavor from the concentrating process. Juices can also be sold in a concentrated state, in which the consumer adds water to the concentrated juice as preparation. The term “extracted fruit juice” according to the invention also encloses such partially processed derivatives from freshly pressed fruit or vegetable juice, like purees, concentrates, dehydrated juices, juice blends, or nectars. Depending on the fruit type the total amount of initial carbohydrates varies over a broad spectrum from e.g. 6 wt.-% total sugar in grapefruit to about 15 wt.-% total sugar in banana, or even 18 wt.-% in grapes. Further details, e.g. the pH values for individual fruits are disclosed in Table 4. Fruit juices have established as a popular beverage choice but it is also used as ingredient in the preparation of several foods. However, due to the relative high total sugar content fruit juices and products derived thereof contribute to malnutrition and obesity. In this respect natural but sugar reduced fruit-based products that fulfill the consumer's expectation of taste are highly desirable. An overview of average non-carbohydrate components (additional ingredients) of extracted fruit juice is given in Table 5.

For the purpose of this invention, “food preparation” describes a man-made mixture as starting virgin liquid nutrient in which the initial carbohydrates are added deliberately for the preparation of higher processed foodstuff, for example, but not limited to, jam, yoghurt, dough and cereals. For the purpose of the specification, food preparations include but are not limited to carbohydrate compositions such as honey or syrup, wherein syrup is preferably derived from starch, grain, rice or vegetable processing (e.g. high fructose corn syrup, rice syrup, grain syrup, barley syrup, or the like). The initial carbohydrates are converted by the in-situ use of enzymes to deliver nutritionally fortified food that fulfils the consumer's expectation with regards to the organoleptic properties but is significantly lower in calorie count and glycemic index compared to conventionally prepared food. Preferably, the food preparation has a water content of at least 10 wt.-%, more preferably at least 50 wt.-%, still more preferably at least 80 wt.-%, in each case relative to the total weight of the food preparation. Thus, the term “liquid” according to the invention also encompasses viscous compositions such as jam, dough and yoghurt. The term “liquid” according to the invention encompasses any composition wherein an enzymatic conversion of an initial carbohydrate into an altered carbohydrate proceeds at an acceptable rate such that satisfactory yields are achieved within days or shorter periods. An overview of average non-carbohydrate components (additional ingredients) of food preparations is given in Table 5.

Preferably, the at least one initial carbohydrate of a virgin liquid nutrient is selected from the group consisting of
for liquid milk: lactose, galactose, and glucose; and/or
for extracted fruit juice: sucrose, inulin, glucose, starch, maltose, and fructose; and/or
for a food preparation: lactose, sucrose, inulin, glucose, galactose, starch, maltose, and fructose.

Preferably, the at least one altered carbohydrate is selected from the group consisting of
for liquid milk: D-allulose, D-mannose, galactose, glucose, fructose, and D-tagatose; and preferably D-allulose, D-mannose, galactose, glucose, and D-tagatose; and more preferably D-allulose, D-mannose, and D-tagatose; and even more preferably D-allulose, and D-tagatose; and most preferably D-allulose; and/or
for extracted fruit juice: nigerose, kojibiose, D-allulose, D-mannose, glucose, fructose, cellobiose, trehalose, IMO, GlucOS, isomaltulose, and DFA III; and preferably nigerose, kojibiose, D-allulose, D-mannose, glucose, fructose cellobiose, IMO, GlucOS, and DFA III; and more preferably nigerose, kojibiose, D-allulose, D-mannose, cellobiose, and DFA III; and most preferably nigerose, kojibiose, and D-allulose; and/or
for a food preparation: DFA III, nigerose, kojibiose, D-allulose, D-tagatose, D-mannose, IMO, GlucOS, isomaltulose, cellobiose, trehalose, galactose, glucose, and fructose; and preferably DFA III, nigerose, kojibiose, D-allulose, D-tagatose, D-mannose, isomaltulose, cellobiose, IMO, GlucOS, and trehalose; and more preferably DFA III, nigerose, kojibiose, D-allulose, D-tagatose, D-mannose, and isomaltulose; and even more preferably DFA III, nigerose, kojibiose, D-allulose, and D-tagatose; and most preferably DFA III, nigerose, kojibiose, D-allulose, D-tagatose, D-mannose, and isomaltulose, and most preferably DFA III, kojibiose, D-allulose.

Preferably, the invention relates to a method for the enzymatic processing of a virgin liquid nutrient comprising one or more initial carbohydrates into a processed liquid nutrient, wherein the altered carbohydrate created is not fructose as the sole conversion product. Equally preferably, the invention relates to a method, in which fructose is a first altered carbohydrate, which undergoes further treatment with one or more enzymes to be completely or partially converted into a second altered carbohydrate.

Preferably, the altered carbohydrate is a disaccharide, selected from the group consisting of
for extracted fruit juice: nigerose, kojibiose, DFA III, cellobiose, trehalose, isomaltose, and isomaltulose; and preferably nigerose, kojibiose, DFA III, cellobiose, and isomaltulose; and more preferably nigerose, kojibiose, cellobiose, and isomaltulose; and most preferably nigerose, and kojibiose; and/or
for a food preparation: DFA III, nigerose, kojibiose, isomaltulose, cellobiose, isomaltose, and trehalose; and preferably DFA III, nigerose, kojibiose, isomaltulose, and cellobiose; and more preferably DFA III, nigerose, kojibiose, and isomaltulose; and even more preferably DFA III, nigerose, and kojibiose; and most preferably DFA III and kojibiose.

Preferably, the enzyme-treated, processed liquid nutrient is characterized
by a reduced glycemic index of at least 5% up to 100%; and/or
by a reduced calorie count of at least 5% up to 100%; and/or
in a comparable textural sensation, and preferably in an identical textural sensation; and/or
in a comparable viscosity or viscoelasticity conferred by the carbohydrates, and preferably in an identical viscosity or viscoelasticity conferred by the carbohydrates; and/or
in a comparable crystallinity conferred by the carbohydrates, and preferably in an identical crystallinity conferred by the carbohydrates
   each and all in comparison to the virgin liquid nutrient.

Preferably, the enzyme-treated, processed liquid nutrient is characterized by
a glycemic index which is reduced by at least 5% up to 100%, at least 10% up to 100%, at least 15% up to 100%, at least 20% up to 100%, at least 25% up to 100%, at least 30% up to 100%, at least 35% up to 100%, at least 40% up to 100%, at least 45% up to 100%, at least 50% up to 100%, at least 55% up to 100%, at least 60% up to 100%, at least 65% up to 100%, at least 70% up to 100%, at least 75% up to 100%, at least 80% up to 100%, or reduced by at least 5% up to 90%, at least 10% up to 90%, at least 15% up to 90%, at least 20% up to 90%, at least 25% up to 90%, at least 30% up to 90%, at least 35% up to 90%, at least 40% up to 90%, at least 45% up to 90%, at least 50% up to 90%, at least 55% up to 90%, at least 60% up to 90%, at least 65% up to 90%, at least 70% up to 90% or reduced by at least 5% up to 80%, at least 10% up to 80%, at least 15% up to 80%, at least 20% up to 80%, at least 25% up to 80%, at least 30% up to 80%, at least 35% up to 80%, at least 40% up to 80%, at least 45% up to 80%, at least 50% up to 80%, at least 55% up to 80%, at least 60% up to 80%, or reduced by at least 5% up to 70%, at least 10% up to 70%, at least 15% up to 70%, at least 20% up to 70%, at least 25% up to 70%, at least 30% up to 70%, at least 35% up to 70%, at least 40% up to 70%, at least 45% up to 70%, at least 50% up to 70%; and/or
a calorie count which is reduced by at least 5% up to 100%, at least 10% up to 100%, at least 15% up to 100%, at least 20% up to 100%, at least 25% up to 100%, at least 30% up to 100%, at least 35% up to 100%, at least 40% up to 100%, at least 45% up to 100%, at least 50% up to 100%, at least 55% up to 100%, at least 60% up to 100%, at least 65% up to 100%, at least 70% up to 100%, at least 75% up to 100%, at least 80% up to 100%, or reduced by at least 5% up to 90%, at least 10% up to 90%, at least 15% up to 90%, at least 20% up to 90%, at least 25% up to 90%, at least 30% up to 90%, at least 35% up to 90%, at least 40% up to 90%, at least 45% up to 90%, at least 50% up to 90%, at least 55% up to 90%, at least 60% up to 90%, at least 65% up to 90%, at least 70% up to 90% or reduced by at least 5% up to 80%, at least 10% up to 80%, at least 15% up to 80%, at least 20% up to 80%, at least 25% up to 80%, at least 30% up to 80%, at least 35% up to 80%, at least 40% up to 80%, at least 45% up to 80%, at least 50% up to 80%, at least 55% up to 80%, at least 60% up to 80%, or reduced by at least 5% up to 70%, at least 10% up to 70%, at least 15% up to 70%, at least 20% up to 70%, at least 25% up to 70%, at least 30% up to 70%, at least 35% up to 70%, at least 40% up to 70%, at least 45% up to 70%, at least 50% up to 70%;
in a comparable textural sensation, and preferably in an identical textural sensation; and/or
in a comparable viscosity or viscoelasticity conferred by the carbohydrates, and preferably in an identical viscosity or viscoelasticity conferred by the carbohydrates; and/or
in a comparable crystallinity conferred by the carbohydrates, and preferably in an identical crystallinity conferred by the carbohydrates
   each and all in comparison to the virgin liquid nutrient.

Preferably, the at least one altered carbohydrate is characterized by at least one, preferably two properties selected from the group consisting of
a glycemic index of from 0% up to 72%, from 0% up to 68%, from 0% up to 60%, from 0% up to 55%, from 0% up to 50%, from 0% up to 45%, from 0% up to 40%, from 0% up to 35%, from 0% up to 32%, from 0% up to 30%, from 0% up to 25%, from 0% up to 20%, from 0% up to 19%, from 0% up to 15%, from 0% up to 10%, from 0% up to 5%, from 0% up to 3%, or from 0% up to 72%, from 3% up to 68%, from 3% up to 60%, from 3% up to 55%, from 3% up to 50%, from 3% up to 45%, from 3% up to 40%, from 3% up to 35%, from 3% up to 32%, from 3% up to 30%, from 3% up to 25%, from 3% up to 20%, from 3% up to 19%, from 3% up to 15%, from 3% up to 10%, from 3% up to 5%, and preferably of from 0% up to 15%, from 0% up to 10%, from 0% up to 5%, from 0% up to 3%, or from 3% up to 15%, from 3% up to 10%, from 3% up to 5%, and most preferably of below 10%; and/or
a calorie count of from 0 kcal/g up 4 kcal/g, from 0 kcal/g up 3.9 kcal/g, from 0 kcal/g up 3.5 kcal/g, from 0 kcal/g up 3 kcal/g, from 0 kcal/g up 2.5 kcal/g, from 0 kcal/g up 2 kcal/g, from 0 kcal/g up 1.7 kcal/g, from 0 kcal/g up 1.5 kcal/g, from 0 kcal/g up 0.3 kcal/g, or from 0.2 kcal/g up 4 kcal/g, from 0.2 kcal/g up 3.9 kcal/g, from 0.2 kcal/g up 3.5 kcal/g, from 0.2 kcal/g up 3 kcal/g, from 0.2 kcal/g up 2.5 kcal/g, from 0.2 kcal/g up 2 kcal/g, from 0.2 kcal/g up 1.7 kcal/g, from 0.2 kcal/g up 1.5 kcal/g, from 0.2 kcal/g up 0.3 kcal/g.

Preferably, the at least one altered carbohydrate is characterized by the following combinations of properties:
a glycemic index of from 0% up to 15%, from 0% up to 10%, from 0% up to 5%, or from 3% up to 15%, from 3% up to 10%, from 3% up to 5%, and most preferably of from 0% up to 5%; and
a calorie count of from 0 kcal/g up 2 kcal/g, from 0 kcal/g up 1.7 kcal/g, from 0 kcal/g up 1.5 kcal/g, from 0 kcal/g up 0.3 kcal/g, or from 0.2 kcal/g up 2 kcal/g, from 0.2 kcal/g up 1.7 kcal/g, from 0.2 kcal/g up 1.5 kcal/g, from 0.2 kcal/g up 0.3 kcal/g.

Preferably, the at least one altered carbohydrate is selected from the group consisting of D-allulose, D-tagatose, nigerose, kojibiose, cellobiose, isomaltose, and/or DFA III.

Preferably, the method is characterized in that in step (iv) the treatment of the virgin liquid nutrient into a processed liquid nutrient with the one or more enzymes occurs
(a) in a one-step process upon simultaneous adding of the one or more enzymes and without interim purification of the partially processed liquid nutrient intermediate; or
(b) in a one-step process upon sequential adding of the one or more enzymes and without interim purification of the partially processed liquid nutrient intermediate; or
(c) in a multi-step process upon sequential adding of the one or more enzymes and with interim purification of the partially processed liquid nutrient intermediate.

Step (ii) of the method according to the invention is optional and may involve adjusting (ii-a) pH value and/or (ii-b) temperature of the virgin liquid nutrient. In preferred embodiments of the invention, the method only involves adjusting (ii-a) pH value, or only involves adjusting (ii-b) temperature, or involves both adjusting (ii-a) pH value and adjusting (ii-b) temperature.

Preferably, the pH value is adjusted to any pH value selected from the group consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, or 9.9; and more preferably selected from the group consisting of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, and even more preferably selected from the group consisting of pH 5.0 to 7.5, pH 3.0 to pH 6.0 pH 4.0 to 7.0, pH 3.5 to 6.5, pH 4.0 to 6.5, and pH 4.5 to 6.5. It is contemplated that after the method the pH value may be readjusted to the original pH value of the virgin liquid nutrient or to another pH value

Suitable additives that may be used in order to adjust the pH value of the virgin liquid nutrient are physiologically acceptable acids and bases including but not limited to mineral acids such as sulfuric acid, phosphorous acid and hydrochloric acid; organic carboxylic acids such as citric acid, ascorbic acid, and lactic acid; inorganic bases such as sodium carbonate, sodium bicarbonate, sodium hydroxide and potassium hydroxide.

Suitable temperatures depend upon the enzymatic conversion and the type of enzymes that are employed. Typical temperatures are within that range of from 5° C. to 70° C.

Step (iii) of the method according to the invention is optional and may involve supplementing

(iii-a) inorganic phosphate and/or
(iii-b) cofactors such as
salts of metal cations (e.g. Fe²⁺, Fe³⁺, Mg²⁺, Mn²⁺, Mn³⁺, Ca²⁺, Co²⁺, Co³⁺, Cu²⁺, Zn²⁺, or Mo²⁺) that are soluble in the virgin liquid nutrient to the virgin liquid nutrient; and/or
ATP, ADP, NAD, NADP, FAD, pyridoxal phosphate, tetrahydrofolic acid, cobalamine, ascorbic acid, coenzyme A, coenzyme Q10, or alpha-liponic acid; and/or
   (iii-c) one or more initial carbohydrates.

In preferred embodiments of the invention, the method only involves supplementing (iii-a) inorganic phosphate, or only involves supplementing (iii-b) cofactors, or only involves supplementing (iii-c) one or more initial carbohydrates.

In preferred embodiments of the invention, the method involves supplementing (iii-a) inorganic phosphate as well as supplementing (iii-b) cofactors, but not supplementing (iii-c) one or more initial carbohydrates; or supplementing (iii-a) inorganic phosphate as well as supplementing (iii-c) one or more initial carbohydrates, but not supplementing (iii-b) cofactors; or supplementing (iii-b) cofactors as well supplementing (iii-c) one or more initial carbohydrates as, but not supplementing (iii-a) inorganic phosphate.

In a preferred embodiment of the invention, the method involves all, supplementing (iii-a) inorganic phosphate as well as supplementing (iii-b) cofactors as well as supplementing (iii-c) one or more initial carbohydrates.

Preferably, inorganic phosphate is supplemented to a final concentration in the virgin liquid nutrient of from 1 mM to 500 mM, from 1 mM to 450 mM, from 1 mM to 400 mM, from 1 mM to 350 mM, from 1 mM to 300 mM, from 1 mM to 250 mM, from 1 mM to 200 mM, from 1 mM to 150 mM, and preferably from 10 mM to 150 mM.

Preferably, inorganic phosphate is supplemented for the formation of the altered carbohydrates trehalose and/or cellobiose. Gentle techniques for removal of inorganic phosphate after completion of the conversion are known in the art, for example removal of inorganic phosphate by electro dialysis.

Preferably, certain mineral salts are supplemented to the virgin liquid nutrient in step (iii-b). Enzymes may require sufficient amounts of certain mineral salts for proper catalytic activity in the course of the method according to the invention. Typically, mineral salts containing for example magnesium ions, manganese ions, cobalt ions, calcium ions, or zinc ions may be supplemented. Preferably, mineral salt ions are supplemented to a final concentration in the virgin liquid nutrient of from 0.01 mM to 25 mM, from 0.1 mM to 10 mM, from 0.5 mM to 10 mM, and preferably from 1 mM to 10 mM.

Preferably, the one or more initial carbohydrates are supplemented in step (iii-c) to the liquid virgin nutrient, e.g. in order to increase the final yield of altered carbohydrate in the obtained processed liquid nutrient. Preferably, supplementation one or more initial carbohydrates is achieved by adding suitable amounts of carbohydrate compositions containing the one or more initial carbohydrates such as honey or syrup, wherein syrup is preferably derived from starch, grain, rice or vegetable processing (e.g. high fructose corn syrup, rice syrup, grain syrup, barley syrup, or the like).

Preferably, in step (iv) the treating of the virgin liquid nutrient with one or more enzymes occurs at a temperature and for reaction times, which are required to convert the virgin liquid nutrient into a processed liquid nutrient, and preferably at a temperature and for reaction times, which are required to reach or approach the thermodynamic equilibrium of the reaction. The thermodynamic equilibrium of the conversion is deemed to be reached, when the composition of initial and altered carbohydrates in the virgin liquid nutrient remains unchanged, despite all enzymes are still catalytically active. It is known by the person skilled in the art how to adjust enzyme activity supplemented to the reaction with reaction time requirements.

Preferably, the method is characterized in treating of the virgin liquid nutrient, i.e. of the at least one initial carbohydrate contained therein, with one or more enzymes in step (iv)
by adding the one and more enzymes to the virgin liquid nutrient, which after conversion of the one or more initial carbohydrates into one or more altered carbohydrates remain part of the processed liquid nutrient and the foodstuff product derived therefrom; and/or
by adding the one and more enzymes to the virgin liquid nutrient, which after conversion of the one or more initial carbohydrates into one or more altered carbohydrates are removed from the processed liquid nutrient or from the foodstuff product derived therefrom; and/or
by adding the one and more enzymes in an immobilized formulation to the virgin liquid nutrient, which after conversion of the one or more initial carbohydrates into one or more altered carbohydrates is removed from the processed liquid nutrient and the foodstuff product derived therefrom by means of column separation; and/or
by contacting the one and more enzymes in an immobilized formulation with the virgin liquid nutrient, for example by column technologies, wherein after conversion of the one or more initial carbohydrates into one or more altered carbohydrates, the processed liquid nutrient and the foodstuff product derived therefrom are released eluted from the column.

In a preferred embodiment of the invention, the one or more enzymes employed in step (iv) are not immobilized.

The principles of enzyme-based processes in industrial technology are well described in the state of the art, as well as knowledge how to modify and design reaction conditions depending on the virgin liquid nutrient to be processed according to the invention. The processes described herein for the production of processed liquid nutrients containing significantly levels of functional carbohydrates and reduced levels of lactose, sucrose, or inulin by treating the virgin liquid nutrient with certain enzymes is carried out for a time, at a pH, and at a temperature effective for converting lactose, sucrose, or inulin as well as the free monosaccharides glucose, and galactose or fructose present as substrates in the virgin liquid nutrient to functional carbohydrates, such as at a temperature of about 10 to about 75° C. for about 0.5 to about 48 hours, preferably at about 30 to about 65° C. for about 0.5 to about 6 hours. The concentration of the initial carbohydrates may vary, but having at least 3 wt.-% lactose if the virgin liquid nutrient is derived from milk. However, if the virgin liquid nutrient is derived from extracted fruit juice the concentration of the disaccharide sucrose and the monosaccharides fructose and glucose may vary over a broad range. For a person skilled in the art the choice and combination of enzymes to be used concomitantly will be driven by the initial carbohydrate composition of the enzymatically untreated virgin liquid nutrient and its foreseen application in the food industry (Table 4).

As will be understood to one of ordinary skill in the art, the order of the steps in the processes described herein, in particular the sets of enzymes applied in combination, can be modified and still obtain a processed liquid nutrient satisfactorily fortified. The processed liquid nutrients fortified according to the invention and products derived thereof also provide health benefits not provided by conventional products. Without intending to limit the scope of the processes described herein, enzyme(s) having functional carbohydrate forming activity may be incorporated into a variety of processes as generally described below. Enzymes useful in the methods described herein include any enzyme preparations known in the art, such as crude preparations, purified enzymes (partially or entirely purified), dried preparations, or enzymes provided as immobilized preparation to enable their easy recovery and recycling. In a preferred aspect, the enzyme treatment is carried out at about 30 to about 65° C. for about 0.5 to about 6 hours. Generally, enzyme(s) having functional carbohydrate forming activity are used at about 25 to about 5000 enzyme units per 100 grams virgin liquid nutrient, preferably about 100 to about 2000 units per 100 grams virgin liquid nutrient. Of course, lesser or greater amounts of enzyme can be used, if desired, and the reaction times may have to be adjusted, as will be readily ascertained by one of ordinary skill in the art, to achieve the desired conversion of the initial carbohydrates to said functional altered carbohydrates. Preferably, the amount of enzymes added to the mixture is selected as an amount that balances the cost of the enzyme and the expense of prolonged enzyme treatment periods.
If the virgin liquid nutrient is derived from milk it can be—after being treated according to the invention—acidified by using any method known in the art either by addition of organic acids or by treating with a lactic acid-producing cultures. Generally, the pH of the milk derived virgin liquid nutrient is lowered to a level of about 4.3 to about 5.2. In addition to the lactose consumed by the functional carbohydrate forming enzymes, a lactose-fermenting culture would also require some lactose as substrate which is consumed by the action of the culture to convert lactose into lactic acid. Therefore, enzyme amount to be used has to be adjusted depending of the targeted dairy product. The same counts if the virgin liquid nutrient is derived from fruits—also here the enzyme amount has to be adjusted depending of the targeted final food product. In the event that the virgin liquid nutrient is a mixture of liquid milk (e.g. yogurt or UHT milk) and extracted fruit juice (e.g. concentrates), the pH of the milk may be lowered to levels of about 3.5 to about 4.0. Depending on the set of enzymes used for the in-situ fortification of the virgin liquid nutrient it can be necessary, especially in case of liquids derived from fruit, to adjust the pH to a range where the selected set of enzymes will be effective.

When the enzyme is mixed with virgin liquid nutrients there is an upper limit to how much enzyme can be used without affecting the sensory properties of the resulting food product. In some preferred embodiments of the invention the enzyme is added in an amount in the range of 1-10,000 ppm. For example, the enzyme may be added to the virgin liquid nutrient in an amount in the range of 1-1000 ppm. The enzyme may e.g. be added to the virgin liquid nutrients in an amount in the range of 1-100 ppm. However, if the enzyme forms part of an enzyme reactor for instance if being immobilized when contacted with the virgin liquid nutrient, a very high enzyme activity may be used, and in such cases the duration of the contact from the enzyme and the virgin liquid nutrient may e.g. be in the range of only 0.1-4 hours. For example, the duration of the contact from the enzyme and the virgin liquid nutrient may be in the range of 0.2-1.5 hours. Alternatively, the duration of the contact from the enzyme and the virgin liquid nutrient may be in the range of 0.1-1 hours, such as in the range of 0.2-0.8 hours. The duration of the contact from the enzyme and the virgin liquid nutrient is preferably sufficient to convert at least 20% of the initial carbohydrates, reducing the starting amount of initial carbohydrates to 80 wt.-%. For example, if the initial carbohydrate content of the virgin liquid nutrient provided was 5.0%, it is thus preferred that at least 20% has been converted into said altered carbohydrates and that at most 80%, corresponding to 4.0 (w/w) initial carbohydrates remain after the enzymatic treatment. Even higher level of initial carbohydrate conversion may be preferred, thus, the duration of the contact from the enzymes and the virgin liquid nutrient may be sufficient to convert at least 80% of the initial carbohydrate of the virgin liquid nutrients and substrates derived thereof. For example, the duration of the contact from the deliberately selected enzymes and the virgin liquid nutrient may e.g. be sufficient to convert at least 90% of the initial carbohydrates. Alternatively, the duration of the contact from the enzymes and the virgin liquid nutrient may e.g. be sufficient to convert at least 95% of the initial carbohydrates. The cooling of the enzyme does not stop its enzymatic activity and that the prolonged storage at low temperature of the virgin liquid nutrient products containing active enzymes may lead to further modifications of the composition. The enzymes may for example be inactivated by heat inactivation e.g. by heating the functional carbohydrate containing milk-derived composition to a temperature of at least 90° C. for at least 10 minutes. Alternatively, further modifications of the composition can be avoided if immobilized enzymes are used. There is plenty of prior art for the immobilization of enzymes and also for the enzymes that are subject matter of the present invention.

It was surprisingly found that the concomitant and/or subsequent combination of certain enzymes in-situ during the gentle processing of virgin liquid nutrients helps to fortify these processed liquid nutrients by the transformation of the main constituting initial carbohydrates,
(i) which in case of milk-based virgin liquid nutrient and liquids derived thereof, like whey, are the disaccharide lactose and the constituent monosaccharides glucose and galactose, and
(ii) which in case of extracted fruit juice as virgin liquid nutrient are the disaccharides sucrose and maltose and the constituent monosaccharides glucose and fructose, and the polysaccharide starch and the constituent monosaccharide glucose, and which
(iii) in case of food preparations are the typically added ingredients sucrose, maltose, fructose, glucose, inulin, starch, and lactose;
   into the altered monomeric and/or dimeric carbohydrates, preferably into D-allulose, D-tagatose, D-mannose, kojibiose, isomaltulose, isomaltose, cellobiose, trehalose, and difructose anhydride (DFA III, alpha-D-fructofuranose, beta-D-fructofuranose 1,2′:2,3′-dianhydride). Moreover, since certain fruits like for example, but not limited to, banana, are rich in the initial carbohydrate inulin, the in-situ use of a fructofuranosidase in such virgin liquid nutrient is suitable to convert inulin into DFA III. Possible initial carbohydrate and altered carbohydrates according to the invention are provided in Table 2 and Table 3. The resulting processed liquid nutrients keep or develop the desired sweetness while the calorie count, and/or glycemic index is significantly reduced, while the textural sensation—mainly characterized by crystallinity and viscosity/viscoelasticity—are maintained to the greatest possible extent. Depending on the enzymes concomitantly and/or subsequently used for the in-situ treatment of the virgin liquid nutrients the resulting processed liquid nutrient and foodstuffs may feature specific ratios of certain altered carbohydrates.

For the purpose of this invention, the processing of a virgin liquid nutrient by one or more enzymes for the conversion of one or more initial carbohydrates into one or more altered carbohydrates according to the invention herein also is briefly referred to as in-situ modification and in-situ fortification of liquid nutrients.

Due to the definition of low-processed foods as well as—in its best meaning—of natural foods the numbers of degrees of freedom in manufacturing are limited. A fortification with functional food ingredients is not obvious by definition and deprives health-conscious consumers from the benefits of such functional ingredients when choosing low-processed foods. Concomitantly, such food cannot be lowered in calories as it would be contradictory to its definition or the targeted market perception. The removal of the fully-caloric carbohydrates, prevailing in the respective food raw materials, would require heavy processing in form of an extraction thereof and substitution of its functionality in terms of taste and texture by admixing certain functional ingredients.

A remedy might be in-situ modification and in-situ fortification of liquid nutrients characterized by the concomitant and combinatorial in-situ use of specific enzymes what offers a new, non-obvious solution to this particular technical problem in the food industry. Its technical character is related to both the underlying process and the obtained products. The processes described herein meet longstanding needs in the art discussed above.

For example, the processes meet the important need for providing significantly reduced calorie count and significantly reduced glycemic index, as well as providing the health benefits of functional carbohydrates with desirable sweet flavor while maintaining desirable organoleptic properties in the final product. The methods described herein reduce the lactose, inulin, fructose, and/or sucrose levels in processed liquid nutrients and products derived thereof by at least about 10%, about 25%, preferably about 30%, more preferably about 35%, more preferably about 40%, yet more preferably at least about 50%, and most preferably even up to about 99%. Even though some prior art exists on this subject matter, the overall state of the art is not very sophisticated. A true remedy would require the combinatorial, concomitant and/or subsequent in-situ use of (engineered) enzymes that would lower the calorie count and/or glycemic index while transforming the naturally contained carbohydrates into altered carbohydrates with significantly lower calorie count and/or significantly lower glycemic index, but still providing a pleasant taste and pleasant textural sensation due to connatural organoleptic properties conferred by the altered carbohydrates if compared to the organoleptic properties conferred by all initial carbohydrates contained in the virgin liquid nutrient.

In this respect, in case of milk or whey as virgin liquid nutrient, lactose and its constituting monosaccharides can be transformed by a more sophisticated and inventive approach into D-allulose, D-tagatose, and/or D-mannose as per layout in FIG. 1.

In this respect, in case of virgin liquid nutrients obtained by the extraction of fruits, the initial carbohydrates sucrose and maltose and their constituting monosaccharides (if not present free) glucose and fructose can be transformed by a more sophisticated and inventive approach into the altered carbohydrates D-allulose, D-mannose, kojibiose, trehalose, cellobiose, IMOs, GlucOS, and isomaltulose as per layout in FIG. 2. Certain fruits like for example, but not limited to, banana, are rich in prebiotic but non-sweet carbohydrate inulin which can be converted by the use of a fructofuranosidase into the low-intensity but very low-caloric sweetener difructose anhydride (DFA III). DFA III is naturally occurring in fructose-rich foodstuff in very low concentrations. DFA III is expected to provide a number of health benefits (Saito and Tomita (2000): Difructose Anhydrides: Their Mass-Production and Physiological Functions. Biosci. Biotechnol. Biochem., Vol. 64(7): 1321-27) why—due to its sweetness and very low calorie count—it is a functional ingredient with high potential.

In case of a food preparation as liquid nutrient all reaction patterns outlined in FIG. 1 and FIG. 2 are applicable. This also applies in case that the starting material of the method is a mixture of one or more virgin liquid nutrients, or a mixture of one or more virgin liquid nutrients with one or more well-defined starting materials, resulting in a “combined” virgin liquid nutrient starting material.

In preferred embodiments of the invention, the virgin liquid nutrient is treated with one enzyme catalyzing one conversion of initial carbohydrates into one or more altered carbohydrates selected from the group consisting of conversions
initial carbohydrate glucose into altered carbohydrate fructose; and/or
initial carbohydrate glucose into altered carbohydrate D-mannose; and/or
initial carbohydrate fructose into altered carbohydrate glucose; and/or
initial carbohydrate fructose into altered carbohydrate D-allulose; and/or
initial carbohydrate fructose into altered carbohydrate D-mannose; and/or
initial carbohydrate inulin into altered carbohydrate DFA III; and/or
initial carbohydrate sucrose into altered carbohydrates fructose and glucose; and/or
initial carbohydrate sucrose into altered carbohydrate kojibiose; and/or
initial carbohydrate sucrose into altered carbohydrate nigerose; and/or
initial carbohydrate sucrose into altered carbohydrate IMO; and/or
initial carbohydrate sucrose into altered carbohydrate GlucOS; and/or
initial carbohydrate sucrose into altered carbohydrate isomaltose; and/or
initial carbohydrate sucrose into altered carbohydrate glucose-1-phosphate; and/or
initial carbohydrate sucrose into altered carbohydrate isomaltulose; and/or
initial carbohydrate galactose into altered carbohydrate D-tagatose; and/or
initial carbohydrate lactose into altered carbohydrates galactose and glucose.

In preferred embodiments of the invention, the virgin liquid nutrient is treated with a first enzyme catalyzing one conversion of initial carbohydrates into one or more first altered carbohydrates, and wherein the one or more first altered carbohydrates is concomitantly treated with one or more additional enzymes catalyzing one or more conversions into a second altered carbohydrate selected from the group consisting of conversions
first altered carbohydrate glucose into second altered carbohydrate D-fructose; and/or
first altered carbohydrate glucose into second altered carbohydrate D-mannose; and/or
first altered carbohydrate fructose into second altered carbohydrate glucose; and/or
first altered carbohydrate fructose into second altered carbohydrate D-allulose; and/or
first altered carbohydrate fructose into second altered carbohydrate D-mannose; and/or
first altered carbohydrate maltose into second altered carbohydrate IMO; and/or
first altered carbohydrate galactose into second altered carbohydrate D-tagatose; and/or
first altered carbohydrate glucose-1-phosphate into second altered carbohydrate cellobiose; and/or
first altered carbohydrate glucose-1-phosphate into second altered carbohydrate cellobiose.

In preferred embodiments of the invention, the one or more first altered carbohydrates is subsequently treated with one or more additional enzymes catalyzing one or more conversions into a second altered carbohydrate selected from the group consisting of conversions of one or more embodiments of the first aspect of this invention. It is within the scope of the invention that the step of converting an initial carbohydrate into a first altered carbohydrate and the second step of converting a first altered carbohydrate into a second altered carbohydrate can be accomplished
(i) in a one-step process upon simultaneous adding of the one or more enzymes for both steps without interim purification of the partially processed liquid nutrient intermediate; or
(ii) in a one-step process upon sequential adding of the one or more enzymes for both steps and without interim purification of the partially processed liquid nutrient intermediate; or
(iii) in a multi-step process upon sequential adding of the one or more enzymes for both steps with interim purification of the partially processed liquid nutrient intermediate.

For the purpose of the invention, fructose is preferably a first altered carbohydrate, which undergoes further at least partial conversion to a second altered carbohydrate. Preferably, the second altered carbohydrate is D-allulose.

In preferred embodiments of the invention, the virgin liquid nutrient is treated with two enzymes catalyzing the conversion of one initial carbohydrate into two or more altered carbohydrates selected from the group consisting of conversions
initial carbohydrate fructose into altered carbohydrates glucose and D-allulose, preferably by use of a glucose-isomerase and a D-psicose-3-epimerase; and/or
initial carbohydrate fructose into altered carbohydrates glucose and D-mannose, preferably by use of a glucose-isomerase and a cellobiose-2-epimerase; and/or
initial carbohydrate fructose into altered carbohydrates D-allulose and D-mannose, preferably by use of a mannose-isomerase and a D-psicose-3-epimerase; and/or
initial carbohydrate lactose into altered carbohydrates galactose and glucose and D-tagatose, preferably by use of a mannose-isomerase and a D-psicose-3-epimerase; and/or
initial carbohydrate sucrose into altered carbohydrates cellobiose and fructose, preferably by use of a sucrose phosphorylase and a cellobiose phosphorylase; and/or
initial carbohydrate sucrose into altered carbohydrates trehalose and fructose, preferably by use of a sucrose phosphorylase and a trehalose phosphorylase; and/or
initial carbohydrate sucrose into altered carbohydrates glucose and D-allulose and fructose, preferably by use of an invertase and a D-psicose-3-epimerase; and/or
initial carbohydrate sucrose into altered carbohydrates glucose and D-mannose and fructose, preferably by use of an invertase and a mannose-isomerase; and/or
initial carbohydrate sucrose into altered carbohydrates fructose and kojibiose, preferably by use of a sucrose phosphorylase and a glucose-isomerase; and/or
initial carbohydrate sucrose into altered carbohydrates fructose and nigerose, preferably by use of a sucrose phosphorylase and a glucose-isomerase
initial carbohydrate sucrose into altered carbohydrates IMOs and D-allulose, preferably by use of a dextransucrase and a D-psicose-3-epimerase; and/or
initial carbohydrate sucrose into altered carbohydrates IMOS and mannose, preferably by use of a dextransucrase and a mannose-isomerase.

In preferred embodiments of the invention, the virgin liquid nutrient is treated with two enzymes catalyzing the conversion of two or more initial carbohydrates into two or more altered carbohydrates selected from the group consisting of conversions
initial carbohydrates fructose and inulin into altered carbohydrates D-allulose and DFA III, preferably by use of a D-psicose-3-epimerase and an inulin fructofuranosidase; and/or
initial carbohydrates fructose and inulin into altered carbohydrates D-mannose and DFA III, preferably by use of a mannose isomerase and an inulin fructofuranosidase; and/or
initial carbohydrates sucrose and inulin into altered carbohydrates isomaltulose and DFA III, preferably by use of a isomaltulose synthase and an inulin fructofuranosidase; and/or
initial carbohydrates sucrose and inulin into altered carbohydrates kojibiose and DFA III, preferably by use of a sucrose phosphorylase and an inulin fructofuranosidase; and/or
initial carbohydrates sucrose and inulin into altered carbohydrates nigerose and DFA III, preferably by use of a sucrose phosphorylase and an inulin fructofuranosidase; and/or
initial carbohydrates sucrose and fructose into altered carbohydrates isomaltulose and D-allulose, preferably by use of an isomaltulose synthase and an D-psicose-3-epimerase; and/or
initial carbohydrates sucrose and fructose into altered carbohydrates kojibiose and D-allulose, preferably by use of a sucrose phosphorylase and an D-psicose-3-epimerase; and/or
initial carbohydrates sucrose and fructose into altered carbohydrates nigerose and D-allulose preferably by use of a sucrose phosphorylase and an D-psicose-3-epimerase; and/or
initial carbohydrates sucrose and fructose into altered carbohydrates isomaltulose and D-mannose, preferably by use of an isomaltulose synthase and a maltose isomerase; and/or
initial carbohydrates sucrose and fructose into altered carbohydrates kojibiose and D-mannose, preferably by use of a sucrose phosphorylase and a maltose isomerase; and/or
initial carbohydrates sucrose and fructose into altered carbohydrates nigerose and D-mannose, preferably by use of a sucrose phosphorylase and a maltose isomerase; and/or
initial carbohydrates sucrose and glucose into altered carbohydrates isomaltulose and fructose, preferably by use of a glucose isomerase and an isomaltose synthase; and/or
initial carbohydrates sucrose and glucose into altered carbohydrates kojibiose and fructose, preferably by use of a sucrose phosphorylase and an isomaltose synthase; and/or
initial carbohydrates sucrose and glucose into altered carbohydrates nigerose and fructose, preferably by use of a sucrose phosphorylase and an isomaltose synthase; and/or
initial carbohydrates sucrose and inulin into altered carbohydrates IMOs and DFA III, preferably by use of a dextransucrase and an inulin fructofuranosidase; and/or
initial carbohydrates lactose and glucose into altered carbohydrates galactose and D-tagatose; and/or
initial carbohydrates lactose and galactose into altered carbohydrates glucose and fructose; and/or
initial carbohydrates glucose and fructose and inulin into altered carbohydrates D-allulose and DFA III; and/or
initial carbohydrates glucose and fructose and inulin into altered carbohydrates D-mannose and DFA III; and/or
initial carbohydrates sucrose and fructose and inulin into altered carbohydrates D-allulose and DFA III; and/or
initial carbohydrates sucrose and fructose and inulin into altered carbohydrates D-mannose and DFA III; and/or
initial carbohydrates sucrose and fructose and inulin into altered carbohydrates isomaltulose and DFA III; and/or
initial carbohydrates sucrose and fructose and inulin into altered carbohydrates kojibiose and DFA III; and/or
initial carbohydrates sucrose and fructose and inulin into altered carbohydrates nigerose and DFA III; and/or
initial carbohydrates sucrose and fructose and inulin into altered carbohydrates isomaltulose and D-allulose; and/or
initial carbohydrates sucrose and fructose and inulin into altered carbohydrates kojibiose and D-allulose; and/or
initial carbohydrates sucrose and fructose and inulin into altered carbohydrates nigerose and D-allulose; and/or
initial carbohydrates sucrose and fructose and inulin into altered carbohydrates isomaltulose and D-mannose; and/or
initial carbohydrates sucrose and fructose and inulin into altered carbohydrates kojibiose and D-mannose; and/or
initial carbohydrates sucrose and fructose and inulin into altered carbohydrates nigerose and D-mannose; and/or
initial carbohydrates sucrose and glucose and inulin into altered carbohydrates isomaltulose and DFA III; and/or
initial carbohydrates sucrose and glucose and inulin into altered carbohydrates kojibiose and DFA III; and/or
initial carbohydrates sucrose and glucose and inulin into altered carbohydrates nigerose and DFA III; and/or
initial carbohydrates sucrose and fructose and glucose into altered carbohydrates isomaltulose and D-allulose; and/or
initial carbohydrates sucrose and fructose and glucose into altered carbohydrates kojibiose and D-allulose; and/or
initial carbohydrates sucrose and fructose and glucose into altered carbohydrates nigerose and D-allulose; and/or
initial carbohydrates sucrose and fructose and glucose into altered carbohydrates isomaltulose and D-mannose; and/or
initial carbohydrates sucrose and fructose and glucose into altered carbohydrates kojibiose and D-mannose; and/or
initial carbohydrates sucrose and fructose and glucose into altered carbohydrates nigerose and D-mannose.

In preferred embodiments of the invention, the virgin liquid nutrient is treated with three and more enzymes catalyzing the conversion of one or more initial carbohydrates into one or more altered carbohydrates selected from the group consisting of conversions
initial carbohydrate sucrose into altered carbohydrates fructose, glucose, D-mannose and D-allulose; and/or
initial carbohydrate sucrose into altered carbohydrates cellobiose and glucose and fructose; and/or
initial carbohydrate sucrose into altered carbohydrates trehalose and glucose and fructose; and/or
initial carbohydrate sucrose into altered carbohydrates kojibiose and D-allulose; and/or
initial carbohydrate sucrose into altered carbohydrates kojibiose and D-mannose; and/or
initial carbohydrate sucrose into altered carbohydrates kojibiose and D-allulose and D-mannose; and/or
initial carbohydrate sucrose into altered carbohydrates nigerose and D-allulose; and/or
initial carbohydrate sucrose into altered carbohydrates nigerose and D-mannose; and/or
initial carbohydrate sucrose into altered carbohydrates nigerose and D-allulose and D-mannose; and/or
initial carbohydrate starch into altered carbohydrate GlucOS; and/or
initial carbohydrate lactose into altered carbohydrates glucose and galactose and fructose and D-tagatose; and/or
initial carbohydrate lactose into altered carbohydrates glucose and galactose and fructose and D-tagatose and D-allulose; and/or
initial carbohydrate lactose into altered carbohydrates glucose and galactose and fructose and D-tagatose and D-mannose; and/or
initial carbohydrate lactose into altered carbohydrates glucose and galactose and fructose and D-tagatose and D-allulose and D-mannose; and/or
initial carbohydrate fructose into altered carbohydrates D-allulose and D-mannose; and/or
initial carbohydrate sucrose and fructose into altered carbohydrates glucose and D-allulose; and/or
initial carbohydrate sucrose and fructose into altered carbohydrates glucose and D-mannose; and/or
initial carbohydrate sucrose and fructose into altered carbohydrates glucose and D-allulose and D-mannose; and/or
initial carbohydrate sucrose and fructose into altered carbohydrates isomaltulose glucose and D-allulose; and/or
initial carbohydrate sucrose and fructose into altered carbohydrates isomaltulose glucose and D-mannose; and/or
initial carbohydrate sucrose and fructose into altered carbohydrates isomaltulose glucose and D-allulose and D-mannose; and/or
initial carbohydrate sucrose and fructose into altered carbohydrates cellobiose and glucose; and/or
initial carbohydrate sucrose and fructose into altered carbohydrates trehalose and glucose; and/or
initial carbohydrate sucrose and fructose into altered carbohydrates kojibiose and D-allulose; and/or
initial carbohydrate sucrose and fructose into altered carbohydrates kojibiose and D-mannose; and/or
initial carbohydrate sucrose and fructose into altered carbohydrates kojibiose and D-allulose and D-mannose; and/or
initial carbohydrate sucrose and fructose into altered carbohydrates nigerose and D-allulose; and/or
initial carbohydrate sucrose and fructose into altered carbohydrates nigerose and D-mannose; and/or
initial carbohydrate sucrose and fructose into altered carbohydrates nigerose and D-allulose and D-mannose; and/or
initial carbohydrate sucrose and glucose into altered carbohydrates fructose and D-allulose; and/or
initial carbohydrate sucrose and glucose into altered carbohydrates fructose and D-mannose; and/or
initial carbohydrate sucrose and glucose into altered carbohydrates fructose and D-allulose and D-mannose; and/or
initial carbohydrate sucrose and glucose into altered carbohydrates isomaltulose and fructose and D-allulose; and/or
initial carbohydrate sucrose and glucose into altered carbohydrates isomaltulose and fructose and D-mannose; and/or
initial carbohydrate sucrose and glucose into altered carbohydrates isomaltulose and fructose and D-allulose and D-mannose; and/or
initial carbohydrate sucrose and glucose into altered carbohydrates cellobiose and fructose; and/or
initial carbohydrate sucrose and glucose into altered carbohydrates trehalose and fructose; and/or
initial carbohydrate sucrose and glucose into altered carbohydrates kojibiose and D-allulose; and/or
initial carbohydrate sucrose and glucose into altered carbohydrates kojibiose and D-mannose; and/or
initial carbohydrate sucrose and glucose into altered carbohydrates kojibiose and D-allulose and D-mannose; and/or
initial carbohydrate sucrose and glucose into altered carbohydrates nigerose and D-allulose; and/or
initial carbohydrate sucrose and glucose into altered carbohydrates nigerose and D-mannose; and/or
initial carbohydrate sucrose and glucose into altered carbohydrates nigerose and D-allulose and D-mannose; and/or
initial carbohydrate sucrose and inulin into altered carbohydrate GlucOS, D-allulose and DFA III; and/or
initial carbohydrate sucrose into altered carbohydrate IMOs, D-allulose and DFA III; and/or
initial carbohydrate sucrose into altered carbohydrate IMOs, D-mannose and DFA III; and/or
initial carbohydrate sucrose and inulin into altered carbohydrates glucose and fructose and D-allulose and DFA III; and/or
initial carbohydrate sucrose and inulin into altered carbohydrates glucose and fructose and D-mannose and DFA III; and/or
initial carbohydrate sucrose and inulin into altered carbohydrates glucose and fructose and D-allulose and D-mannose and DFA III; and/or
initial carbohydrate sucrose and inulin into altered carbohydrates isomaltulose and glucose and fructose and D-allulose and DFA III; and/or
initial carbohydrate sucrose and inulin into altered carbohydrates isomaltulose and glucose and fructose and D-mannose and DFA III; and/or
initial carbohydrate sucrose and inulin into altered carbohydrates isomaltulose and glucose and fructose and D-allulose and D-mannose and DFA III; and/or
initial carbohydrate sucrose and inulin into altered carbohydrates cellobiose and glucose and fructose and DFA III; and/or
initial carbohydrate sucrose and inulin into altered carbohydrates trehalose and glucose and fructose and DFA III; and/or
initial carbohydrate sucrose and inulin into altered carbohydrates kojibiose and D-allulose and DFA III; and/or
initial carbohydrate sucrose and inulin into altered carbohydrates kojibiose and D-mannose and DFA III; and/or
initial carbohydrate sucrose and inulin into altered carbohydrates kojibiose and D-allulose and D-mannose and DFA III; and/or
initial carbohydrate sucrose and inulin into altered carbohydrates nigerose and D-allulose and DFA III; and/or
initial carbohydrate sucrose and inulin into altered carbohydrates nigerose and D-mannose and DFA III; and/or
initial carbohydrate sucrose and inulin into altered carbohydrates nigerose and D-allulose and D-mannose and DFA III; and/or
initial carbohydrate sucrose and inulin into altered carbohydrates kojibiose and fructose and DFA III; and/or
initial carbohydrate sucrose and inulin into altered carbohydrates nigerose and fructose and DFA III; and/or
initial carbohydrate sucrose and fructose and inulin into altered carbohydrates glucose and D-allulose and DFA III; and/or
initial carbohydrate sucrose and fructose and inulin into altered carbohydrates glucose and D-mannose and DFA III; and/or
initial carbohydrate sucrose and fructose and inulin into altered carbohydrates glucose and D-allulose and D-mannose and DFA III; and/or
initial carbohydrate sucrose and fructose and inulin into altered carbohydrates isomaltulose and D-allulose and DFA III; and/or
initial carbohydrate sucrose and fructose and inulin into altered carbohydrates isomaltulose and D-mannose and DFA III; and/or
initial carbohydrate sucrose and fructose and inulin into altered carbohydrates isomaltulose and D-allulose and D-mannose and DFA III; and/or
initial carbohydrate sucrose and fructose and inulin into altered carbohydrates cellobiose and glucose and DFA III; and/or
initial carbohydrate sucrose and fructose and inulin into altered carbohydrates trehalose and glucose and DFA III; and/or
initial carbohydrate sucrose and fructose and inulin into altered carbohydrates kojibiose and D-allulose and DFA III; and/or
initial carbohydrate sucrose and fructose and inulin into altered carbohydrates kojibiose and D-mannose and DFA III; and/or
initial carbohydrate sucrose and fructose and inulin into altered carbohydrates kojibiose and D-allulose and D-mannose and DFA III; and/or
initial carbohydrate sucrose and fructose and inulin into altered carbohydrates nigerose and D-allulose and DFA III; and/or
initial carbohydrate sucrose and fructose and inulin into altered carbohydrates nigerose and D-mannose and DFA III; and/or
initial carbohydrate sucrose and fructose and inulin into altered carbohydrates nigerose and D-allulose and D-mannose and DFA III; and/or
initial carbohydrate sucrose and fructose and inulin into altered carbohydrates kojibiose and glucose and DFA III; and/or
initial carbohydrate sucrose and fructose and inulin into altered carbohydrates nigerose and glucose and DFA III; and/or
initial carbohydrate sucrose and glucose and inulin into altered carbohydrates fructose and D-allulose and DFA III; and/or
initial carbohydrate sucrose and glucose and inulin into altered carbohydrates fructose and D-mannose and DFA III; and/or
initial carbohydrate sucrose and glucose and inulin into altered carbohydrates fructose and D-allulose and D-mannose and DFA III; and/or
initial carbohydrate sucrose and glucose and inulin into altered carbohydrates isomaltulose and fructose and D-allulose and DFA III; and/or
initial carbohydrate sucrose and glucose and inulin into altered carbohydrates cellobiose and fructose and DFA III; and/or
initial carbohydrate sucrose and glucose and inulin into altered carbohydrates trehalose and fructose and DFA III; and/or
initial carbohydrate sucrose and glucose and inulin into altered carbohydrates kojibiose and D-allulose and DFA III; and/or
initial carbohydrate sucrose and glucose and inulin into altered carbohydrates kojibiose and D-mannose and DFA III; and/or
initial carbohydrate sucrose and glucose and inulin into altered carbohydrates kojibiose and D-allulose and D-mannose and DFA III; and/or
initial carbohydrate sucrose and glucose and inulin into altered carbohydrates nigerose and D-allulose and DFA III; and/or
initial carbohydrate sucrose and glucose and inulin into altered carbohydrates nigerose and D-mannose and DFA III; and/or
initial carbohydrate sucrose and glucose and inulin into altered carbohydrates nigerose and D-allulose and D-mannose and DFA III; and/or
initial carbohydrate sucrose and glucose and inulin into altered carbohydrates kojibiose and fructose and DFA III; and/or
initial carbohydrate sucrose and glucose and inulin into altered carbohydrates nigerose and fructose and DFA III; and/or
initial carbohydrate lactose and galactose into altered carbohydrates glucose and fructose and D-tagatose; and/or
initial carbohydrate lactose and galactose into altered carbohydrates glucose and fructose and D-tagatose and D-allulose; and/or
initial carbohydrate lactose and galactose into altered carbohydrates glucose and fructose and D-tagatose and D-mannose; and/or
initial carbohydrate lactose and galactose into altered carbohydrates glucose and fructose and D-tagatose and D-allulose and D-mannose; and/or
initial carbohydrate lactose and glucose into altered carbohydrates galactose and fructose and D-tagatose; and/or
initial carbohydrate lactose and glucose into altered carbohydrates galactose and fructose and D-tagatose and D-allulose; and/or
initial carbohydrate lactose and glucose into altered carbohydrates galactose and fructose and D-tagatose and D-mannose; and/or
initial carbohydrate lactose and glucose into altered carbohydrates galactose and fructose and D-tagatose and D-allulose and D-mannose.

Lactose-containing liquid milk and other dairy substrates derived thereof are contacted with enzyme(s) having activities effective for converting the initial carbohydrates lactose, galactose and glucose to said altered functional carbohydrates. The processes described herein can reduce lactose in the dairy products to by far less than about 1 gram per serving, an amount that can be tolerated by most lactose-intolerant individuals. The products provided herein are nutritionally-enhanced products containing functional carbohydrates while at the same time still having excellent organoleptic properties with desired texture and flavor. Health promoting benefits are for example, but not limited to, due to a reduced glycemic index since the functional altered carbohydrates are more slowly absorbed and/or metabolized than lactose or its hydrolysis products and/or having less calories and/or are acting as prebiotics on the intestinal flora.

Preferably the lactose-containing liquid milk and/or other dairy substrates derived thereof are mixed with one or more other virgin liquid nutrients, or with certain well-defined starting materials such as carbohydrate compositions prior to the enzymatic treating according to the invention. In dairy product manufacturing, often sweeteners are admixed in order to increase the sweetness of the final product. Such added sweeteners can also include initial carbohydrates of the virgin liquid nutrient, and may be converted into altered carbohydrates in accordance with the invention. Admixed sweetener sources are carbohydrate compositions such as honey or syrup, wherein syrup is preferably derived from starch, grain, rice or vegetable processing (e.g. high fructose corn syrup, rice syrup, grain syrup, barley syrup, or the like).

In a preferred embodiment of the invention, the disaccharide lactose in liquid milk is in-situ hydrolyzed into its monosaccharides glucose and galactose by the use of enzymes named beta-galactosidase. If L-arabinose isomerase is in-situ applied in the virgin liquid nutrient, galactose is converted into D-tagatose. Additionally, or optionally, the released glucose gets in-situ isomerized to D-mannose by using enzymes like for example a cellobiose-2-epimerase. Additionally, or optionally, the released glucose gets in-situ isomerized to fructose and subsequently converted to D-allulose by using enzymes named glucose isomerase and D-psicose-3-epimerase and/or converted to D-mannose by using enzymes named glucose isomerase and mannose isomerase. The enzymes are used in-situ and concomitantly either as free or immobilized enzymes. In one preferred embodiment, the resulting product obtained according to the invention contains fructose, glucose, and D-allulose in a defined ratio of approx. 1.2:1.0:0.4. Levels of residual lactose are below 0.2 to 1.0%. The calorie count of the respective product is preferably reduced by approx. 5 to 10% compared to the calorie count of the starting material. The glycemic index of the respective product is also reduced by 10 to 15% compared to the glycemic index of the starting material. Preferably, additional sucrose, glucose, or fructose may be added to the liquid milk, increasing the ratio of D-allulose upon conversion according to the invention.

In a preferred embodiment of the invention, the resulting product obtained according to the invention contains galactose, D-tagatose, fructose, glucose, and D-allulose in a specified ratio of preferably approx. 1.3:0.9:1.2:1.0:0.4. Levels of residual lactose are below 0.5 to 1.0%. The calorie count of the respective product is preferably reduced by approx. 20 to 30% compared to the calorie count of the starting material. The glycemic index of the respective product is preferably reduced by approx. 20 to 40% compared to the glycemic index of the starting material. In an even more preferred embodiment, the resulting product obtained according to the invention contains galactose, D-tagatose, fructose, glucose, D-allulose, and D-mannose in a specified ratio of preferably approx. 1.5:1.3:0,9:0.9:1.0:0.3:0.3. Levels of residual lactose are below 0.5 to 1.0%. The calorie count of the respective product is preferably reduced by approx. 20 to 30% compared to the calorie count of the starting material. The glycemic index of the respective product is preferably reduced by 25 to 45% compared to the glycemic index of the starting material.

According to the invention, the initial carbohydrates sucrose, glucose, and fructose contained in virgin liquid nutrients obtained by extraction from fruit(s) can be converted in several functional carbohydrates by the in-situ use of enzymes according to the invention.

In a preferred embodiment of the invention, the disaccharide sucrose is hydrolyzed by enzymes named invertase to increase the levels of glucose and fructose. The released fructose is in-situ converted to D-allulose by using an enzyme like the D-psicose-3-epimerase. Additionally, and optionally, the resulting glucose gets in-situ isomerized to fructose and further converted to D-allulose by said D-psicose-3-epimerase. The resulting product obtained according to the invention contains, fructose and D-allulose in a specified ratio of preferably approx. 1.0:0.4. Levels of residual sucrose are preferably below 0.5%. The calorie count of the respective product is preferably reduced by approx. 15% compared to the calorie count of the starting material. The glycemic index of the respective product is preferably reduced by approx. 10 to 40% compared to the glycemic index of the starting material.

In a preferred embodiment of the invention, the disaccharide sucrose is hydrolyzed by enzymes named invertase to increase the levels of glucose and fructose. The released glucose is in-situ converted to D-mannose by using an enzyme like the cellobiose-2-epimerase. Additionally, and optionally, the released fructose gets in-situ isomerized to glucose and further converted to D-mannose by said cellobiose-2-epimerase. The resulting product obtained according to the invention contains, glucose and D-mannose in a specified ratio of preferably approx. 1.0:0.2. Levels of residual sucrose are preferably below 0.5%. The calorie count of the respective product is preferably reduced by approx. 5% compared to the calorie count of the starting material. The glycemic index of the respective product is preferably reduced by approx. 10% compared to the glycemic index of the starting material.

In a preferred embodiment of the invention, the disaccharide sucrose is hydrolyzed by enzymes named invertase to increase the levels of glucose and fructose. The released fructose is in-situ converted to D-allulose by using an enzyme like the D-psicose 3-epimerase. Additionally, and optionally, the released fructose gets in-situ isomerized to D-mannose by using an enzyme like mannose isomerase. The resulting product obtained according to the invention contains, fructose and D-allulose in a specified ratio of preferably approx. 1.0:0.4 fructose and D-mannose in a specified ratio of preferably approx. 1.0:0.4. Levels of residual sucrose are preferably below 0.5%. The calorie count of the respective product is preferably reduced by approx. 15 to 20% compared to the calorie count of the starting material. The glycemic index of the respective product is preferably reduced by approx. 10 to 40% compared to the glycemic index of the starting material.

It is known to the person skilled in the art, that the formation of D-allulose and/or D-mannose is also possible if no invertase or glucose isomerase enzymes are used in case that free fructose and/or glucose are present in the virgin liquid nutrient. If the hydrolysis of sucrose is omitted, the above-mentioned ratios from the monosaccharides are not affected. If no glucose isomerase is used, the ratio from fructose and glucose is ruled by their initial content and whether sucrose gets hydrolyzed or not, while the ratios from fructose/D-allulose and/or fructose/D-mannose and/or glucose/D-mannose remain constant. All of the above-mentioned enzymes are used in-situ and concomitantly either as free or immobilized enzymes.

If the initially contained sucrose is not or only partially hydrolyzed it can be used for the formation of functional carbohydrates, namely the disaccharides isomaltulose, trehalose, cellobiose, kojibiose, and/or nigerose.

In a preferred embodiment of the invention, the disaccharide sucrose converted by dextransucrase enzymes to increase the levels of IMOs and fructose in the processed liquid nutrient. Optionally, the released fructose is further in-situ converted into D-allulose by using an enzyme like the D-psicose 3-epimerase. Additionally, and optionally, the released fructose gets in-situ isomerized to D-mannose by using an enzyme like mannose isomerase.

In a preferred embodiment of the invention, the initial carbohydrate sucrose gets in-situ isomerized to isomaltulose by using an enzyme called isomaltulose synthase. Depending on the amount of sucrose in the starting virgin liquid nutrient and the amount of other carbohydrates contained, the glycemic index is preferably reduced by up to 50% while the calorie count and sweetness remain the same. Another preferred embodiment is given if the virgin liquid nutrient contains free fructose and if besides isomaltulose synthase also a D-psicose-3-epimerase is used. The calorie count and glycemic index can be further reduced by up to 30% related to the amount of free fructose due to the formation of D-allulose. A further preferred embodiment is given if the virgin liquid nutrient contains free fructose and free glucose and if besides isomaltulose synthase also a D-psicose-3-epimerase and a glucose isomerase is used. The calorie count and glycemic index can be further reduced by up to 30% related to the amount of free fructose due to the formation of D-allulose. The calorie count and glycemic index can be reduced by up to 15% related to the amount of free glucose due to the formation of fructose and subsequent formation of D-allulose.

In a preferred embodiment of the invention, the initial carbohydrate sucrose gets in-situ converted according to the invention to trehalose by using an enzyme called sucrose phosphorylase and trehalose phosphorylase. In one another preferred embodiment, the initial carbohydrate sucrose gets in-situ converted to cellobiose by using an enzyme called sucrose phosphorylase and cellobiose phosphorylase. The formation of trehalose and/or cellobiose requires the addition of inorganic phosphate in concentrations of 10 to 150 mM which after completion of the reaction is removed for instance by electrodialysis. Electrodialysis is a standard technique in food processing and highly suitable for the removal of inorganic phosphate (Mikhaylin and Bazinet (2009): Electrodialysis in Food Processing. Reference Module in Food Science, 1-6, Elsevier). If in a further preferred embodiment the aforementioned enzymes are combined with a glucose isomerase, an almost complete conversion of sucrose is possible. Related to the initial amount of sucrose in the virgin liquid nutrient the calorie count and glycemic index can be reduced up to 95% each. A further preferred embodiment is given if besides sucrose free fructose is contained and if besides the aforementioned enzymes a D-psicose-3-epimerase is used. The calorie count and glycemic index can be further reduced by up to 30% related to the amount of free fructose due to the formation of D-allulose. A further preferred embodiment is given if the virgin liquid nutrient contains free fructose and free glucose and if besides the aforementioned enzymes a glucose isomerase is used. The calorie count and glycemic index can be further reduced by up to 15% related to the amount of free glucose due to the formation of fructose and subsequent formation of D-allulose.

In a preferred embodiment of the invention, the initial carbohydrate sucrose and glucose get in-situ converted to the disaccharides kojibiose and/or nigerose by using a sucrose phosphorylase, which at low levels of inorganic phosphate but excess amounts of glucose, transfers the glucose moiety of sucrose to free glucose under formation of kojibiose and/or nigerose depending on the used enzyme. Depending on the amount of sucrose in the starting virgin liquid nutrient, the calorie count can be reduced by up to 50% and glycemic index is preferably reduced by up to. 50%. Depending on the amount of glucose in the starting virgin liquid nutrient, the calorie count can be further reduced by up to 50% and glycemic index is preferably reduced by up to. 50%. In principle, the enzymatic conversion of sucrose into kojibiose by the sucrose phosphorylase covers the transfer of a moiety of the sucrose onto a glucose co-substrate thereby releasing kojibiose and fructose as side product. Certain virgin liquid nutrients, such as extracted fruit juice, by nature contain sufficient amounts of glucose to enable efficient conversion, however, certain virgin liquid nutrients will need supplementation of glucose as additional initial carbohydrate.

In consideration of the enzymatic conversion of sucrose into kojibiose requiring appropriate concentrations of glucose and of inorganic phosphate, the enzymatic conversion in step (iv) according to the invention may require the adjustment of certain additional substrates or reaction conditions, such reaction conditions can be established by corresponding supplementation in step (iii) or the invention. Accordingly, in a preferred embodiment of the invention, the virgin liquid nutrient is supplemented in step (iii-c) with glucose from external source as a cofactor for enzymatic conversion of the initial carbohydrate sucrose into the altered carbohydrates kojibiose and the side product fructose. The concentration to be adjusted is described in literature and known to the person skilled in the art.

In a preferred embodiment of the invention, the initial carbohydrate sucrose and fructose get in-situ converted to the disaccharides kojibiose and/or nigerose by using a glucose isomerase which transforms fructose to glucose and a sucrose phosphorylase, which at low levels of inorganic phosphate but excess amounts of glucose, transfers the glucose moiety of sucrose to free glucose under formation of kojibiose and/or nigerose depending on the used enzyme. Depending on the amount of sucrose in the starting virgin liquid nutrient, the calorie count can be reduced by up to 50% and glycemic index is preferably reduced by up to. 50%. Depending on the amount of fructose in the starting virgin liquid nutrient, the calorie count can be further reduced by up to 25% and glycemic index is preferably reduced by up to. 25%.

In a preferred embodiment of the invention, the initial carbohydrate sucrose and fructose get in-situ converted to the disaccharides kojibiose and/or nigerose and D-allulose by using a glucose isomerase which transforms fructose to glucose and a mannose isomerase that transforms fructose to D-allulose and a sucrose phosphorylase which at low levels of inorganic phosphate but excess amounts of glucose transfers the glucose moiety of sucrose to free glucose under formation of kojibiose and/or nigerose depending on the used enzyme. Depending on the amount of sucrose in the starting virgin liquid nutrient, the calorie count can be reduced by up to 50% and glycemic index is preferably reduced by up to. 50%. Depending on the amount of fructose in the starting virgin liquid nutrient, the calorie count can be further reduced by up to 40% and glycemic index is preferably reduced by up to. 40%.

In a preferred embodiment of the invention, the initial carbohydrate sucrose and glucose get in-situ converted to the disaccharides kojibiose and/or nigerose and D-allulose by using a glucose isomerase which transforms fructose to glucose and a mannose isomerase that transforms fructose to D-allulose and a sucrose phosphorylase which at low levels of inorganic phosphate but excess amounts of glucose transfers the glucose moiety of sucrose to free glucose under formation of kojibiose and/or nigerose depending on the used enzyme. Depending on the amount of sucrose in the starting virgin liquid nutrient, the calorie count can be reduced by up to 50% and glycemic index is preferably reduced by up to. 50%. Depending on the amount of glucose in the starting virgin liquid nutrient, the calorie count can be further reduced by up to 50% and glycemic index is preferably reduced by up to. 50%.

According to the invention, in another preferred embodiment of the invention, the prebiotic poly- and oligosaccharide inulin naturally contained in fruits is in-situ converted into difructose anhydride (DFA III) by the use of the enzyme fructofuranosidase. The enzyme is used in-situ and concomitantly either as free or immobilized enzyme. In one preferred embodiment, more than 25% of the naturally contained inulin is transformed into difructose anhydride, in another preferred embodiment more than 50% of the contained inulin is transformed into difructose anhydride. The calorie count of the respective product remains almost the same while the sweetness is tremendously improved. If inulin is present with a concentration of 1 wt.-% and if 50% of it are converted into DFA III a sweetness enhancement is achieved that equals the addition of 0.35% sucrose (w/w).

In a preferred embodiment of the invention, the virgin liquid nutrient is liquid milk, wherein in step (iii-c) according to the invention fructose is supplemented as initial carbohydrate, and wherein step (iv) involves the enzymatic conversion of at least a portion of the fructose into D-allulose. Preferably, the supplemented fructose may be added as a purified or partially purified carbohydrate, or as a component in a mixture of an extracted fruit juice, or of a derivative thereof, or of a food preparation, e.g. from any syrup or honey. More preferably, other initial carbohydrates contained in the liquid milk, or in a supplemented mixture of an extracted fruit juice, of a derivative thereof, or of a food preparation, are also enzymatically converted into an altered carbohydrate, for example sucrose into kojibiose, or lactose into glucose and galactose and further secondary altered carbohydrates derived thereof.

In a preferred embodiment of the invention, the virgin liquid nutrient is extracted fruit juice, wherein the initial carbohydrate is sucrose, and wherein step (iv) involves the enzymatic conversion of at least a portion of the sucrose into kojibiose. Optionally, glucose may be supplemented in step (iii) of the invention as co-substrate of the enzymatic conversion. Preferably, the extracted fruit juice contains further fructose as initial carbohydrate, or additional fructose that is supplemented as a purified or partially purified carbohydrate in step (iii-c), or additional fructose added as a component in mixing the extracted fruit juice with a food preparation virgin liquid nutrient, e.g. with any syrup or honey, and wherein such added fructose is converted into an altered carbohydrate, i.e. into D-allulose.

In a preferred embodiment of the invention, the virgin liquid nutrient is a mixture of one or more virgin liquid nutrients selected from the group of liquid milk, extracted fruit juice, and food preparation, and preferably from liquid milk and extracted fruit juice, or from a food preparation and extracted juice.

In a preferred embodiment of the invention, the virgin liquid nutrient is a mixture of one or more virgin liquid nutrients selected from the group of liquid milk, extracted fruit juice, and food preparation, and preferably from liquid milk and extracted fruit juice, or from a food preparation and extracted juice, and the method is characterized by converting the at least one initial carbohydrate contained in such a mixture of virgin liquid nutrients with one or more enzymes in step (iv), wherein the one or more enzymes are not immobilized.

For all of the above-mentioned embodiments of the first aspect aiming at the alteration of the initial carbohydrate sucrose into a functional disaccharide, further embodiments can be deduced by the additional and optional use of enzymes that transform glucose to D-mannose and in order to further valorize the starting virgin liquid nutrient. In one another preferred embodiment of the invention the virgin liquid nutrient derived from extracted fruit juice is treated with isomaltulose-synthase and D-psicose-3-epimerase and cellobiose-2-epimerase resulting.

For a person skilled in the art the choice and combination of enzymes to be used concomitantly will be driven by the initial carbohydrate composition of the enzymatically untreated virgin liquid nutrient (Table 4). For a person skilled in the art it is also obvious that the processes described herein are also applicable if the starting virgin liquid nutrient is used as an ingredient for the preparation of higher processed foodstuff, for example, but not limited to, jam, yoghurt, dough, cereals, or bread. The invention also applies to mixtures of nutrients and ingredients in which lactose, sucrose, fructose and/or inulin are added as ingredients in order to obtain a food preparation for which all of the above and even more embodiments can be applied. Furthermore, the invention also applies to mixtures of a virgin liquid nutrient with additional virgin liquid nutrients and/or other well-defined ingredient starting materials e.g. carbohydrate composition such as honey or syrup, wherein syrup is preferably derived from starch, grain, rice or vegetable processing (e.g. high fructose corn syrup, rice syrup, grain syrup, barley syrup, or the like), increasing the ratio of a specific initial carbohydrate and after conversion according to the invention, resulting in an increased content of the corresponding altered carbohydrates for which all of the above and even more embodiments can be applied. Preferably, the invention applies for the mixing of liquid milk with extracted fruit juice and/or with additional preparations containing sucrose, glucose, or fructose (e.g. honey, high fructose corn syrup, rice syrup, grain syrup, or barley syrup), resulting in an increased D-allulose content upon conversion according to the invention.

In preferred embodiments of the invention, the enzymes for treatment of the virgin liquid nutrient in step (iv) are selected from the groups consisting of
enzymes from EC classes EC 5.1.3.30, EC 5.1.3.31, EC 5.3.1.4, EC 5.3.1.5, EC 3.2.1.1, EC 3.2.1.2, EC 3.2.1.41, EC 3.2.1.26, EC 3.2.1.11, EC 3.2.1.94, EC 5.3.1.7, EC 3.2.1.23, EC 2.4.1.5, EC 2.4.1.7, EC 2.4.1.64, EC 2.4.1.20, EC 2.4.1.24, EC 5.1.3.11, and EC 4.2.2.18; and/or
enzymes with the name D-psicose-3-epimerase (EC 5.1.3.30), D-tagatose-3-epimerase (EC 5.1.3.31), invertase (or beta-fructofuranosidase, EC 3.2.1.26), L-arabinose-isomerase (EC 5.3.1.4), dextransucrase (EC 2.4.1.5), glucansucrase (2.4.1.5, 2.4.1.140), alpha-amylase (EC 3.2.1.1), beta-amylase (EC 3.2.1.2), pullulanase (EC 3.2.1.41), alpha-transglucosidase (EC 2.4.1.24), dextranase (EC 3.2.1.11, EC 3.2.1.94), glucose-isomerase (EC 5.3.1.5), mannose-isomerase (EC 5.3.1.7), beta-galactosidase (EC 3.2.1.23), sucrose phosphorylase (EC 2.4.1.7), trehalose phosphorylase (EC 2.4.1.64), cellobiose phosphorylase (EC 2.4.1.20), cellobiose-2-epimerase (EC 5.1.3.11), and inulin fructotransferase (EC 4.2.2.18)
   and for each group enclosing both, the wild-type enzymes as well as improved enzyme variants obtained by improved enzyme obtained by engineering.

In a preferred embodiment of the invention, the enzymes for treatment of the virgin liquid nutrient in step (iv) are selected from the groups consisting of enzymes from EC classes EC 5.1.3.30, EC 5.1.3.31, EC 5.3.1.4, EC 5.3.1.5, EC 3.2.1.1, EC 3.2.1.2, EC 3.2.1.41, EC 3.2.1.26, EC 3.2.1.11, EC 3.2.1.94, EC 5.3.1.7, EC 3.2.1.23, EC 2.4.1.5, EC 2.4.1.7, EC 2.4.1.64, EC 2.4.1.20, EC 2.4.1.24, EC 5.1.3.11, and EC 4.2.2.18.

In a preferred embodiment of the invention, the enzymes for treatment of the virgin liquid nutrient in step (iv) are selected from the groups consisting of enzymes with the names D-psicose-3-epimerase (belonging to the enzyme group with EC class number EC 5.1.3.30), D-tagatose-3-epimerase (belonging to the enzyme group with EC class number EC 5.1.3.31), invertase (or beta-fructofuranosidase, belonging to the enzyme group with EC class number EC 3.2.1.26), L-arabinose-isomerase (belonging to the enzyme group with EC class number EC 5.3.1.4), dextransucrase (belonging to the enzyme group with EC class number EC 2.4.1.5), glucansucrase (belonging to the enzyme group with EC class numbers 2.4.1.5, or 2.4.1.140), alpha-amylase (belonging to the enzyme group with EC class number EC 3.2.1.1), beta-amylase (belonging to the enzyme group with EC class number EC 3.2.1.2), pullulanase (belonging to the enzyme group with EC class number EC 3.2.1.41), alpha-transglucosidase (belonging to the enzyme group with EC class number EC 2.4.1.24), dextranase (belonging to the enzyme group with EC class numbers EC 3.2.1.11, or EC 3.2.1.94), glucose-isomerase (belonging to the enzyme group with EC class number EC 5.3.1.5), mannose-isomerase (belonging to the enzyme group with EC class number EC 5.3.1.7), beta-galactosidase (belonging to the enzyme group with EC class number EC 3.2.1.23), sucrose phosphorylase (belonging to the enzyme group with EC class number EC 2.4.1.7), trehalose phosphorylase (belonging to the enzyme group with EC class number EC 2.4.1.64), cellobiose phosphorylase (belonging to the enzyme group with EC class number EC 2.4.1.20), cellobiose-2-epimerase (belonging to the enzyme group with EC class number EC 5.1.3.11), and inulin fructotransferase (belonging to the enzyme group with EC class number EC 4.2.2.18).

In a preferred embodiment, the enzymes for treatment of the virgin liquid nutrient in step (iv) are naturally occurring enzymes (also referred to as “wild-type enzymes”) and/or variants of naturally occurring enzymes obtained by engineering, which variants usually are characterized by improved enzyme characteristics.

The improvement of enzymes can be achieved by enzyme engineering. This technique involves the development of variants of a starting enzyme sequence with improved properties (for review: S. Lutz, U. T. Bornscheuer, Protein Engineering Handbook, Wiley VCH, Weinheim, 2009).

In a preferred embodiment, the enzymes for treatment of the virgin liquid nutrient in step (iv) are naturally occurring enzymes and/or variants thereof selected from the groups consisting of D-psicose-3-epimerase belonging to the EC class EC 5.1.3.30, and D-tagatose-3-epimerases belonging to the EC class EC 5.1.3.31, and catalyze the enzymatic conversion of fructose into allulose. It is known in the art, that enzyme candidates of the D-psicose-3-epipmerase enzyme family and the D-tagatose-3-epimerase enzyme family catalyze the epimerization of various ketoses at the C3 position, including the ketoses D-tagatose, D-ribulose, D-xylulose, D-sorbose, D-fructose, and D-psicose, however, each naturally occurring enzyme has a slightly different substrate spectrum for different ketoses, as described for example in Zhang L. et al., Biotechnol. Left (2009) 31:857-862. Examples of naturally occurring enzymes suitable for catalyzing the conversion of D-fructose into D-allulose include, without limitation, D-psicose-3-epimerase enzymes or D-tagatose-3-epimerases enzymes derived from the species Pseudomonas sp., Agrobacterium sp., Rhizobium sp., Clostridium sp., Flavonifractor sp., Ruminococcus sp., Anaerostipes sp., Thermotoga sp., Mesorhizobium sp., Desmospora sp., Rhodobactor sp., Arthobactor sp., Burkholderia sp. Specifically, naturally occurring enzymes suitable for catalyzing the conversion of D-fructose into D-allulose have been described from the organisms summarized in Table 6.1, which enzymes will be suitable for converting fructose into D-allulose during treatment of the virgin liquid nutrient in step (iv) according to the invention, which enzymes are herein made part of the disclosure of the invention. In addition, all naturally occurring enzymes and engineered variants thereof as being described in the patents or patent applications of Table 6.1 are herein made part of the disclosure of the invention.

Equally preferred, the enzymes for treatment of the virgin liquid nutrient in step (iv) are selected from the groups consisting of naturally occurring sucrose phosphorylases belonging to EC class EC 2.4.1.7 and/or variants thereof and catalyze the enzymatic conversion of sucrose into kojibiose. It is known in the art, that enzyme candidates of this sucrose phosphorylase family catalyze the conversion of sucrose into kojibiose, however, in many cases with a low efficiency only. Examples of naturally occurring enzymes suitable for catalyzing the conversion of sucrose into kojibiose include, without limitation, sucrose phosphorylases derived from the species Leuconostoc sp., and Bifidobacterium sp. Specifically, naturally occurring enzymes suitable for catalyzing the conversion of sucrose into kojibiose are described from Leconostoc mesenteroides, and Bifidobacterium adolescens. It is known in the art, that the efficiency of converting sucrose into kojibiose can be improved by enzyme engineering: for example, the European Patent EP 3224370 discloses certain sequence positions of the naturally occurring sucrose phosphorylase enzyme from Bifidobacterium adolescens, being disclosed as SEQ ID NO:1, which upon substitution increase the efficiency of the formation of kojibiose from sucrose. Table 6.2 summarizes naturally occurring enzymes and engineered variants derived thereof, which are known to be capable of converting sucrose into kojibiose during treatment of the virgin liquid nutrient in step (iv) according to the invention, which enzymes are herein made part of the disclosure of the invention. It is also within the scope of the invention, that variants of the naturally occurring enzymes of Table 6.2, which carry one or more of the following substitutions, corresponding to substitutions in sequence positions P134V, P134R, P134W, P134S, R135E, A193G, H234T, L341I, L343P, Y344R, Y344D, Y344V, Y344I, Q345S, Q345N of the SEQ ID NO:1 being disclosed in EP 3224370, are used in according to the invention. In addition, the sucrose phosphorylase enzyme variants described in EP 3224370 B1 as variants of the application's SEQ ID NO:1 with one or more of the following substitutions P134V, P134R, P134W, P134S, R135E, A193G, H234T, L341I, L343P, Y344R, Y344D, Y344V, Y344I, Q345S, Q345N outlined in Table 6.2 are made part of the disclosure of the invention.

In a preferred embodiment of the invention, the enzymes for treatment of the virgin liquid nutrient in step (iv) are enzymes, which comprise an amino acid sequence of at least 70% identity, more preferably at least 75% identity, still more preferably at least 80% identity, yet more preferably at least 85% identity, even more preferably at least 90% identity, or at least 91% identity, or at least 92% identity, or at least 93% identity, or at least 94% identity, most preferably at least 95% identity, or at least 96% identity, or at least 97% identity, and in particular at least 98% identity, or at least 99% identity to the naturally occurring enzymes and/or variants thereof selected from the groups consisting of D-psicose-3-epimerase belonging to the EC class EC 5.1.3.30, and D-tagatose-3-epimerases belonging to the EC class EC 5.1.3.31, being disclosed in Table 6.1, which catalyze the enzymatic conversion of fructose into allulose according to the invention.

In a preferred embodiment of the invention, the enzymes for treatment of the virgin liquid nutrient in step (iv) are enzymes, which comprise an amino acid sequence of at least 70% identity, more preferably at least 75% identity, still more preferably at least 80% identity, yet more preferably at least 85% identity, even more preferably at least 90% identity, or at least 91% identity, or at least 92% identity, or at least 93% identity, or at least 94% identity, most preferably at least 95% identity, or at least 96% identity, or at least 97% identity, and in particular at least 98% identity, or at least 99% identity to the naturally occurring enzymes and/or variants thereof selected from the sucrose phosphorylases belonging to EC class EC 2.4.1.7, being disclosed in Table 6.2, which catalyze the enzymatic conversion of sucrose into kojibiose according to the invention.

It is known how the identity and homology, respectively, of a polymer of amino acid residues is determined. For the purpose of this invention, homology and identity are understood as synonyms. Percent identity is calculated as: Sequence Identity [%]=number of Matches/L×100, wherein L is the number of aligned positions, i.e. identities and nonidentities (including gaps, if any). Identity is preferably calculated using BLASTP (see, for example, Altschul S F et al. (1997) “Gapped BLAST and PSI-BLAST: a new generation of protein database search programs”, Nucleic Acids Res. 25:3389-3402; or Altschul SF (2005) “Protein database searches using compositionally adjusted substitution matrices.” FEBS J. 272:5101-5109), preferably with the following algorithm parameters: Matrix: BLOSUM62; Gap Costs: Existence: 11 Extension: 1, Expect threshold: 10 and Word size: 6. Results are filtered for sequences with more than 35% query coverage. BlastP can be accessed online at the NCBI Homepage (https://blast.ncbi.nlm.nih.gov/Blast.cgi?PROGRAM=blastp&PAGE TYPE=BlastSearch&LINKLOC=blasthome). Other program setting can be adjusted as desired, for example using the following settings:
Field “Enter Query Sequence”: Query subrange: none
Field “Choose Search Set”: Database: non-redundant protein sequences (nr); optional parameters: none
Field “Program Selection”: Algorithm: blastp (protein-protein BLAST)
Algorithm parameters: Field “General parameters”: Max target sequences: 20000; Short queries: Automatically adjust parameters for short input sequences; Expect threshold: 10; Word size: 6; Max matches in a query range: 0
Algorithm parameters: Field “Scoring parameters”: Matrix: BLOSUM62; Gap Costs: Existence: 11 Extension: 1; Compositional adjustments: Conditional compositional score matrix adjustment
Algorithm parameters: Field “Filters and Masking”: Filter: none; Mask: none

TABLE 6.1
D-psicose-3-empimerase enzymes and D-tagatose-3-epimerase
enzymes suitable for enzymatic conversion of fructose
into D-allulose in step (iv) of the invention
Enzymes from Databases

| | Genbank number // |
|---|---|
| Organism | NCBI accession number |
| Desmospora sp. 8437 | EGK07060.1 |
| Agrobacterium tumefaciens | AAK88700 |
| Clostridium cellulolyticum H10 | ACL75304 |
| Clostridium scindens ATCC 35704 | EDS06411.1 |
| Clostridium hylemonae DSM 15053 | EDS06411.1 |
| Flavonifractor plautii | EHM40452.1 |
| Ensifer adhaerens | AUF32146.1 |
| Dorea sp. CAG317 | CDD07088.1 |
| Treponema primita | WP_010256447.1 |
| Clostridium sp. BNL1100 | AEY67409.1 |
| Ruminococcus sp. 5_1_39BFAA | EES75522.1 |
| Clostridium bolteae ATCC BAA-613 | EDP19602.1 |
| Pseudomonas cichorii | BAA24429 |
| Rhodobacter sphaeroides SK011 | ACO59490 |
| Arthrobacter globiformis M30 | AB981957 |
| Arthrobacter sp. ATCC 31749 | AECL01000014.1 |
| Rhizobium leguminosarum | AFO04175.1 |
| Burkholderia multivorans CGD1 | EEE02543.1 |
| Anaerostipes caccae | EDR98778.1 |
| Thermotoga maritima MSB8 | AAD35501.1 |
| Mesorhizobium loti | BAB50456.1 |

Enzymes from Patent Literature

WO2011/040708, WO 2013/027999, WO 2014/049373, WO 2015/
032761, WO 2016/191267, WO 2017/081666, WO 2018/116266,
US 2011/0275138, U.S. Pat. No. 9,988,618 B2, EP 2990483,
CN 106148311, CN 108239632, CN 108239633, CN 108018278,
CN 106418427, CN 103849612, CN 103849613, CN 106350498,
CN 103710330, CN 103789378, CN 103789377

TABLE 6.2
Sucrose phosphorylase enzymes suitable for enzymatic conversion
of sucrose into kojibiose in step (iv) of the invention
Enzymes from Databases

| | Genebank number // |
|---|---|
| Organism | NCBI accession number |
| Leuconostoc mesenteroides | AAX33736.1 GI:60678803 |
| Bifidobacterium adolescentis | WP_011742626.1 |

Enzymes from Patent Literature

Sucrose phosphorylase enzyme variants described in EP 3224370 B1
as variants of the application's SEQ ID NO: 1 carrying one or
more of the following substitutions P134V, P134R, P134W, P134S,
R135E, A193G, H234T, L341I, L343P, Y344R, Y344D, Y344V,
Y344I, Q345S, Q345N are made part of the disclosure of the invention.

Another aspect of the invention pertains to the use of an enzyme as described herein in the method according to the invention, i.e. for the enzymatic processing of a virgin liquid nutrient comprising one or more initial carbohydrates into a processed liquid nutrient comprising one or more altered carbohydrates.

In a preferred embodiment of the invention, the virgin liquid nutrient in step (iv) is treated with one or more enzymes for the conversion of one or more initial carbohydrates into one or more altered carbohydrates, wherein the enzyme is characterized by one or more functional features (A), (B), (C), (D), (E),
(A) a catalytic activity for carbohydrate forming in the virgin liquid nutrient of at least 1 to 5000 enzyme units per 100 grams virgin liquid nutrient, at least 25 to 5000 enzyme units per 100 grams virgin liquid nutrient, and preferably about 100 to about 2000 units per 100 grams virgin liquid nutrient;
(B) a high catalytic activity at the pH of the virgin liquid nutrient selected from the group consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, or 9.9; and more preferably selected from the group consisting of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, and even more preferably selected from the group consisting of pH 5.0 to 7.5, pH 3.0 to pH 6.0, pH 4.0 to 7.0, pH 3.5 to 6.5, pH 4.0 to 6.5, and pH 4.5 to 6.5;
(C) a high process stability in the environment of the virgin liquid nutrient expressed at thermal stability for from 1 hour up to 672 hours, from 1 hour up to 500 hours, from 1 hour up to 400 hours, from 1 hour up to 300 hours, from 1 hour up to 200 hours, from 1 hour up to 168 hours, from 1 hour up to 144 hours, from 1 hour up to 120 hours, from 1 hour up to 96 hours, from 1 hour up to 72 hours, from 1 hour up to 48 hours, from 1 hour up to 24 hours, from 1 hour up to 12 hours, or from 1 hour up to 6 hours;
(D) a high activity at high concentrations of one or more initial carbohydrates of from 0.5 to 70 wt.-%, from 0.5 to 65 wt.-%, from 0.5 to 60 wt.-%, from 0.5 to 55 wt.-%, from 0.5 to 50 wt.-%, from 0.5 to 45 wt.-%, from 0.5 to 40 wt.-%, from 0.5 to 35 wt.-%, from 0.5 to 30 wt.-%, from 0.5 to 25 wt.-%, from 0.5 to 20 wt.-%, or from 0.5 to 15 wt.-%; or from 1 to 70 wt.-%, from 1 to 65 wt.-%, from 1 to 60 wt.-%, from 1 to 55 wt.-%, from 1 to 50 wt.-%, from 1 to 45 wt.-%, from 1 to 40 wt.-%, from 1 to 35 wt.-%, from 1 to 30 wt.-%, from 1 to 25 wt.-%, from 1 to 20 wt.-%, or from 1 to 15 wt.-%; or from 3 to 70 wt.-%, from 3 to 65 wt.-%, from 3 to 60 wt.-%, from 3 to 55 wt.-%, from 3 to 50 wt.-%, from 3 to 45 wt.-%, from 3 to 40 wt.-%, from 3 to 35 wt.-%, from 3 to 30 wt.-%, from 3 to 25 wt.-%, from 3 to 20 wt.-%, or from 3 to 15 wt.-%;
(E) a high activity at high concentrations of one or more altered carbohydrates of from 5 to 70 wt.-%, from 5 to 65 wt.-%, from 5 to 60 wt.-%, from 5 to 55 wt.-%, from 5 to 50 wt.-%, from 5 to 45 wt.-%, from 5 to 40 wt.-%, from 5 to 35 wt.-%, from 5 to 30 wt.-%, from 5 to 25 wt.-%, from 5 to 20 wt.-%, or from 5 to 15 wt.-%; or from 10 to 70 wt.-%, from 10 to 65 wt.-%, from 10 to 60 wt.-%, from 10 to 55 wt.-%, from 10 to 50 wt.-%, from 10 to 45 wt.-%, from 10 to 40 wt.-%, from 10 to 35 wt.-%, from 10 to 30 wt.-%, from 10 to 25 wt.-%, from 10 to 20 wt.-%, or from 10 to 15 wt.-%; or from 15 to 70 wt.-%, from 15 to 65 wt.-%, from 15 to 60 wt.-%, from 15 to 55 wt.-%, from 15 to 50 wt.-%, from 15 to 45 wt.-%, from 15 to 40 wt.-%, from 15 to 35 wt.-%, from 15 to 30 wt.-%, from 15 to 25 wt.-%, or from 15 to 20 wt.-%.

In a preferred embodiment of the invention, the virgin liquid nutrient in step (iv) is treated with one or more engineered enzymes for the conversion of one or more initial carbohydrates into one or more altered carbohydrates, wherein the enzyme is characterized by an improvement compared to the corresponding wild-type enzyme on one or more functional features (A), (B), (C), (D), (E),
(A) a catalytic activity for carbohydrate forming in the virgin liquid nutrient of at least 1 to 5000 enzyme units per 100 grams virgin liquid nutrient, at least 25 to 5000 enzyme units per 100 grams virgin liquid nutrient, and preferably about 100 to about 2000 units per 100 grams virgin liquid nutrient;
(B) a high catalytic activity at the pH of the virgin liquid nutrient selected from the group consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, or 9.9; and more preferably selected from the group consisting of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, and even more preferably selected from the group consisting of pH 5.0 to 7.5, pH 3.0 to pH 6.0, pH 4.0 to 7.0, pH 3.5 to 6.5, pH 4.0 to 6.5, and pH 4.5 to 6.5;
(C) a high process stability in the environment of the virgin liquid nutrient expressed at thermal stability for from 1 hour up to 672 hours, from 1 hour up to 500 hours, from 1 hour up to 400 hours, from 1 hour up to 300 hours, from 1 hour up to 200 hours, from 1 hour up to 168 hours, from 1 hour up to 144 hours, from 1 hour up to 120 hours, from 1 hour up to 96 hours, from 1 hour up to 72 hours, from 1 hour up to 48 hours, from 1 hour up to 24 hours, from 1 hour up to 12 hours, or from 1 hour up to 6 hours;
(D) a high activity at high concentrations of one or more initial carbohydrates of from 0.5 to 70 wt.-%, from 0.5 to 65 wt.-%, from 0.5 to 60 wt.-%, from 0.5 to 55 wt.-%, from 0.5 to 50 wt.-%, from 0.5 to 45 wt.-%, from 0.5 to 40 wt.-%, from 0.5 to 35 wt.-%, from 0.5 to 30 wt.-%, from 0.5 to 25 wt.-%, from 0.5 to 20 wt.-%, or from 0.5 to 15 wt.-%; or from 1 to 70 wt.-%, from 1 to 65 wt.-%, from 1 to 60 wt.-%, from 1 to 55 wt.-%, from 1 to 50 wt.-%, from 1 to 45 wt.-%, from 1 to 40 wt.-%, from 1 to 35 wt.-%, from 1 to 30 wt.-%, from 1 to 25 wt.-%, from 1 to 20 wt.-%, or from 1 to 15 wt.-%; or from 3 to 70 wt.-%, from 3 to 65 wt.-%, from 3 to 60 wt.-%, from 3 to 55 wt.-%, from 3 to 50 wt.-%, from 3 to 45 wt.-%, from 3 to 40 wt.-%, from 3 to 35 wt.-%, from 3 to 30 wt.-%, from 3 to 25 wt.-%, from 3 to 20 wt.-%, or from 3 to 15 wt.-%;
(E) a high activity at high concentrations of one or more altered carbohydrates of from 5 to 70 wt.-%, from 5 to 65 wt.-%, from 5 to 60 wt.-%, from 5 to 55 wt.-%, from 5 to 50 wt.-%, from 5 to 45 wt.-%, from 5 to 40 wt.-%, from 5 to 35 wt.-%, from 5 to 30 wt.-%, from 5 to 25 wt.-%, from 5 to 20 wt.-%, or from 5 to 15 wt.-%; or from 10 to 70 wt.-%, from 10 to 65 wt.-%, from 10 to 60 wt.-%, from 10 to 55 wt.-%, from 10 to 50 wt.-%, from 10 to 45 wt.-%, from 10 to 40 wt.-%, from 10 to 35 wt.-%, from 10 to 30 wt.-%, from 10 to 25 wt.-%, from 10 to 20 wt.-%, or from 10 to 15 wt.-%; or from 15 to 70 wt.-%, from 15 to 65 wt.-%, from 15 to 60 wt.-%, from 15 to 55 wt.-%, from 15 to 50 wt.-%, from 15 to 45 wt.-%, from 15 to 40 wt.-%, from 15 to 35 wt.-%, from 15 to 30 wt.-%, from 15 to 25 wt.-%, or from 15 to 20 wt.-%.

In a preferred embodiment of the invention, the virgin liquid nutrient in step (iv) is liquid milk and is treated with one or more enzymes characterized by one or more functional features (A), (B), (C), (D),
(A) high catalytic activity in the environment of the liquid milk, namely (i) being active in biphasic milk emulsion, and/or (ii) being active at calcium ion (Ca²) concentrations of 10 mg/100 mL up to 200 mg/mL, or 20 mg/100 mL up to 200 mg/mL, or 40 mg/100 mL up to 200 mg/mL, or 60 mg/100 mL up to 200 mg/mL, or 80 mg/100 mL up to 200 mg/mL, or 100 mg/100 mL up to 200 mg/mL, or 125 mg/100 mL up to 200 mg/mL, or 150 mg/100 mL up to 200 mg/mL;
(B) high catalytic activity at the pH of the liquid milk, namely at pH from 3.5 to 8.5, from pH from 4.0 to 8.5, from 4.5 to 8.0, from 5.0 to 8.0, and preferably from pH 5.0 to 7.5;
(C) high process stability in the environment of the liquid milk, namely being stable in biphasic milk emulsions;
(D) high activity at low concentrations of one or more initial carbohydrates, namely at lactose concentration of at least 3.0 wt.-%, and glucose and/or galactose concentrations of at least 1.0 wt.-% each.

In a preferred embodiment of the invention, the virgin liquid nutrient in step (iv) is extracted fruit juice and is treated with one or more enzymes characterized by one or more functional features (A), (B), (C), (D), (E),
(A) high catalytic activity in the environment of the extracted fruit juice;
(B) high catalytic activity at the pH of the extracted fruit juice, namely from pH 1.0 to pH 8.0, from pH 2.0 to pH 7.0, from pH 2.5 to pH 7.5, and preferably from pH 3.0 to pH 6.0;
(C) high process stability in the environment of the extracted fruit juice;
(D) high activity at low concentrations of one or more initial carbohydrates, namely sucrose, glucose, fructose concentration of at least 1.0 wt.-%;
(E) high activity at high concentrations of one or more altered carbohydrates, namely at concentrations higher 10 wt.-% of sucrose, fructose, or glucose as they occur naturally or in concentrated extracted fruit juice.

In a preferred embodiment of the invention, the virgin liquid nutrient in step (iv) is a food preparation and is treated with one or more enzymes characterized by one or more functional features (A), (B), (C), (D), (E),
(A) high catalytic activity in the environment of the food preparation;
(B) high catalytic activity at the pH of the food preparation, namely pH from 3.5 to 8.5, from pH from 4.0 to 8.5, from 4.5 to 8.0, from 5.0 to 8.0, from pH 5.0 to 7.5, and preferably from pH 4.0 to 7.0;
(C) high process stability in the environment of the food preparation;
(D) high activity at low concentrations of one or more initial carbohydrates, namely sucrose, glucose, fructose concentration of at least 1.0 wt.-%;
(E) high activity at high concentrations of one or more altered carbohydrates, namely at concentrations higher 10 wt.-% of sucrose, fructose, glucose, and/or inulin as they occur naturally in ingredients used in the food preparation or added as such to the food preparation.

In a preferred embodiment of the invention, the one or more enzymes that after treatment of the virgin liquid nutrient in step (iv) are contained in the processed liquid nutrient and are inactivated; preferably (a) by heat treatment of the processed liquid nutrient; (b) by shift of the pH value to a pH in which the enzymes are inactive, (c) by treatment of the processed liquid nutrient with protease enzymes; and/or (d) by supplementation of inhibitory chemical substances, preferably mineral salts, into the processed liquid nutrient. Specific methods for the inactivation of enzymes in complex mixtures are well known to persons skilled in the art. Heat inactivation is often realized by short-term incubation at 95° C., however, for sensitive processed liquid nutrients, inactivation may be accomplished at lower temperature and longer incubation time. In addition, a shift in pH of the processed virgin liquid may help to reduce or eliminate the enzymatic activity. Proteases could be used for degradation of the enzymes according to the invention; preferably, proteases that are being used and allowed as food-compatible processing aids could be used for this purpose. Supplementation of inhibitory compounds according to the invention must be in accordance with regulatory requirements of the processed liquid nutrient; examples for possibly non-critical inhibitors may be mineral salts, like calcium or magnesium ions, which reduce or abolish the catalytic activity. According to prevailing legislation, an inactivation of the enzymes may not be required, but may be desired in order to avoid further conversions in the processed liquid nutrient or any subsequent product derived therefrom.

In a preferred embodiment of the invention, the virgin liquid nutrient in step (iv) is treated with one or more enzymes described in any one of the previous embodiments herein.

In a preferred embodiment of the invention, the processed liquid nutrient is used as an ingredient for mixing with other food ingredients, further processing or confectioning, or for preparation of a food preparation.

In a preferred embodiment of the invention, which is also an embodiment of any previous embodiments, the processed liquid nutrient is characterized by
a glycemic index of all initial carbohydrates and altered carbohydrates contained in the processed liquid nutrient, which is lower than the glycemic index of all initial carbohydrates contained in the virgin liquid nutrient; and/or
a calorie count of all initial carbohydrates and altered carbohydrates contained in the processed liquid nutrient, which is lower than the calorie count of all initial carbohydrates contained in the virgin liquid nutrient; and/or
the textural sensation conferred by all initial carbohydrates and altered carbohydrates contained in the processed liquid nutrient are connatural compared to the textural sensation conferred by all initial carbohydrates contained in the virgin liquid nutrient; and/or
the sweetness conferred by all initial carbohydrates and altered carbohydrates contained in the processed liquid nutrient are connatural compared to the sweetness conferred by all initial carbohydrates contained in the virgin liquid nutrient.

In a preferred embodiment of the invention, which is also an embodiment of any previous embodiments, the processed liquid nutrient is characterized by
a glycemic index which is reduced by at least 5% up to 100%, at least 10% up to 100%, at least 15% up to 100%, at least 20% up to 100%, at least 25% up to 100%, at least 30% up to 100%, at least 35% up to 100%, at least 40% up to 100%, at least 45% up to 100%, at least 50% up to 100%, at least 55% up to 100%, at least 60% up to 100%, at least 65% up to 100%, at least 70% up to 100%, at least 75% up to 100%, at least 80% up to 100%, or reduced by at least 5% up to 90%, at least 10% up to 90%, at least 15% up to 90%, at least 20% up to 90%, at least 25% up to 90%, at least 30% up to 90%, at least 35% up to 90%, at least 40% up to 90%, at least 45% up to 90%, at least 50% up to 90%, at least 55% up to 90%, at least 60% up to 90%, at least 65% up to 90%, at least 70% up to 90% or reduced by at least 5% up to 80%, at least 10% up to 80%, at least 15% up to 80%, at least 20% up to 80%, at least 25% up to 80%, at least 30% up to 80%, at least 35% up to 80%, at least 40% up to 80%, at least 45% up to 80%, at least 50% up to 80%, at least 55% up to 80%, at least 60% up to 80%, or reduced by at least 5% up to 70%, at least 10% up to 70%, at least 15% up to 70%, at least 20% up to 70%, at least 25% up to 70%, at least 30% up to 70%, at least 35% up to 70%, at least 40% up to 70%, at least 45% up to 70%, at least 50% up to 70%; and/or
a calorie count which is reduced by at least 5% up to 100%, at least 10% up to 100%, at least 15% up to 100%, at least 20% up to 100%, at least 25% up to 100%, at least 30% up to 100%, at least 35% up to 100%, at least 40% up to 100%, at least 45% up to 100%, at least 50% up to 100%, at least 55% up to 100%, at least 60% up to 100%, at least 65% up to 100%, at least 70% up to 100%, at least 75% up to 100%, at least 80% up to 100%, or reduced by at least 5% up to 90%, at least 10% up to 90%, at least 15% up to 90%, at least 20% up to 90%, at least 25% up to 90%, at least 30% up to 90%, at least 35% up to 90%, at least 40% up to 90%, at least 45% up to 90%, at least 50% up to 90%, at least 55% up to 90%, at least 60% up to 90%, at least 65% up to 90%, at least 70% up to 90% or reduced by at least 5% up to 80%, at least 10% up to 80%, at least 15% up to 80%, at least 20% up to 80%, at least 25% up to 80%, at least 30% up to 80%, at least 35% up to 80%, at least 40% up to 80%, at least 45% up to 80%, at least 50% up to 80%, at least 55% up to 80%, at least 60% up to 80%, or reduced by at least 5% up to 70%, at least 10% up to 70%, at least 15% up to 70%, at least 20% up to 70%, at least 25% up to 70%, at least 30% up to 70%, at least 35% up to 70%, at least 40% up to 70%, at least 45% up to 70%, at least 50% up to 70%;
in a comparable textural sensation, and preferably in an identical textural sensation; and/or
in a comparable viscosity or viscoelasticity conferred by the carbohydrates, and preferably in an identical viscosity or viscoelasticity conferred by the carbohydrates; and/or
in a comparable crystallinity conferred by the carbohydrates, and preferably in an identical crystallinity conferred by the carbohydrates
   each and all in comparison to the virgin liquid nutrient.

In a second aspect, the invention relates to a processed liquid nutrient which is obtainable by the method according to the invention as described herein, i.e. manufactured according to the first aspect of the invention and/or any of the embodiments of the first aspect, wherein preferably
a glycemic index of all initial carbohydrates and altered carbohydrates contained in the processed liquid nutrient, is lower than the glycemic index of all initial carbohydrates contained in the virgin liquid nutrient; and/or
a calorie count of all initial carbohydrates and altered carbohydrates contained in the processed liquid nutrient, is lower than the calorie count of all initial carbohydrates contained in the virgin liquid nutrient; and/or
a textural sensation conferred by all initial carbohydrates and altered carbohydrates contained in the processed liquid nutrient, is connatural compared to the textural sensation conferred by all initial carbohydrates contained in the virgin liquid nutrient; and/or
a sweetness conferred by all initial carbohydrates and altered carbohydrates contained in the processed liquid nutrient, is connatural compared to the sweetness conferred by all initial carbohydrates contained in the virgin liquid nutrient.

In a preferred embodiment of the invention, the textural sensation conferred by all initial carbohydrates and altered carbohydrates contained in the processed liquid nutrient and the textural sensation conferred by all initial carbohydrates contained in the virgin liquid nutrient are expressed as
the viscosity or viscoelasticity conferred by all the carbohydrates, wherein the viscosity or viscoelasticity conferred by all initial carbohydrates and altered carbohydrates of the processed liquid nutrient in comparison to the viscosity or viscoelasticity conferred by all initial carbohydrates contained in the virgin liquid nutrient is connatural; and/or
the crystallinity conferred by all the carbohydrates, wherein the crystallinity conferred by all initial carbohydrates and altered carbohydrates of the processed liquid nutrient in comparison to the crystallinity conferred by all initial carbohydrates contained in the virgin liquid nutrient is connatural.

In a preferred embodiment of the invention, the processed liquid nutrient is characterized by
a glycemic index which is reduced by at least 5% up to 100%, at least 10% up to 100%, at least 15% up to 100%, at least 20% up to 100%, at least 25% up to 100%, at least 30% up to 100%, at least 35% up to 100%, at least 40% up to 100%, at least 45% up to 100%, at least 50% up to 100%, at least 55% up to 100%, at least 60% up to 100%, at least 65% up to 100%, at least 70% up to 100%, at least 75% up to 100%, at least 80% up to 100%, or reduced by at least 5% up to 90%, at least 10% up to 90%, at least 15% up to 90%, at least 20% up to 90%, at least 25% up to 90%, at least 30% up to 90%, at least 35% up to 90%, at least 40% up to 90%, at least 45% up to 90%, at least 50% up to 90%, at least 55% up to 90%, at least 60% up to 90%, at least 65% up to 90%, at least 70% up to 90% or reduced by at least 5% up to 80%, at least 10% up to 80%, at least 15% up to 80%, at least 20% up to 80%, at least 25% up to 80%, at least 30% up to 80%, at least 35% up to 80%, at least 40% up to 80%, at least 45% up to 80%, at least 50% up to 80%, at least 55% up to 80%, at least 60% up to 80%, or reduced by at least 5% up to 70%, at least 10% up to 70%, at least 15% up to 70%, at least 20% up to 70%, at least 25% up to 70%, at least 30% up to 70%, at least 35% up to 70%, at least 40% up to 70%, at least 45% up to 70%, at least 50% up to 70%; and/or
a calorie count which is reduced by at least 5% up to 100%, at least 10% up to 100%, at least 15% up to 100%, at least 20% up to 100%, at least 25% up to 100%, at least 30% up to 100%, at least 35% up to 100%, at least 40% up to 100%, at least 45% up to 100%, at least 50% up to 100%, at least 55% up to 100%, at least 60% up to 100%, at least 65% up to 100%, at least 70% up to 100%, at least 75% up to 100%, at least 80% up to 100%, or reduced by at least 5% up to 90%, at least 10% up to 90%, at least 15% up to 90%, at least 20% up to 90%, at least 25% up to 90%, at least 30% up to 90%, at least 35% up to 90%, at least 40% up to 90%, at least 45% up to 90%, at least 50% up to 90%, at least 55% up to 90%, at least 60% up to 90%, at least 65% up to 90%, at least 70% up to 90% or reduced by at least 5% up to 80%, at least 10% up to 80%, at least 15% up to 80%, at least 20% up to 80%, at least 25% up to 80%, at least 30% up to 80%, at least 35% up to 80%, at least 40% up to 80%, at least 45% up to 80%, at least 50% up to 80%, at least 55% up to 80%, at least 60% up to 80%, or reduced by at least 5% up to 70%, at least 10% up to 70%, at least 15% up to 70%, at least 20% up to 70%, at least 25% up to 70%, at least 30% up to 70%, at least 35% up to 70%, at least 40% up to 70%, at least 45% up to 70%, at least 50% up to 70%;
a comparable textural sensation, and preferably by an identical textural sensation; and/or
by a comparable viscosity or viscoelasticity conferred by the carbohydrates, and preferably by an identical viscosity or viscoelasticity conferred by the carbohydrates; and/or
by a comparable crystallinity conferred by the carbohydrates, and preferably by an identical crystallinity conferred by the carbohydrates
   each and all in comparison to the virgin liquid nutrient.

In a preferred embodiment of the invention, the processed liquid nutrient is characterized by containing one or more altered carbohydrates selected from the group consisting of
for liquid milk: D-allulose, D-mannose, galactose, glucose, fructose, and D-tagatose, and preferably D-allulose, D-mannose, galactose, glucose, and D-tagatose, and more preferably D-allulose, D-mannose, and D-tagatose, and even more preferably D-allulose, and D-tagatose and most preferably D-allulose; and/or
for extracted fruit juice: nigerose, kojibiose, D-allulose, D-mannose, glucose, fructose cellobiose, trehalose, isomaltulose, IMOs, GlucOS, isomaltose, and DFA III, and preferably nigerose, kojibiose, D-allulose, D-mannose, glucose, fructose cellobiose, IMOs, GlucOS, isomaltose, and DFA III, and more preferably nigerose, kojibiose, D-allulose, D-mannose, cellobiose, and DFA III, and most preferably nigerose, kojibiose, and D-allulose and preferably nigerose, kojibiose, D-mannose, D-allulose, DFA III, cellobiose, trehalose, and isomaltulose and; and/or
for a food preparation: DFA III, nigerose, kojibiose, D-allulose, D-tagatose, D-mannose, isomaltulose, cellobiose, trehalose, galactose, glucose, and fructose, IMOs, GlucOS, isomaltose, and preferably DFA III, nigerose, kojibiose, D-allulose, D-tagatose, D-mannose, isomaltulose, IMOs, GlucOS, isomaltose, cellobiose, and trehalose, and more preferably DFA III, nigerose, kojibiose, D-allulose, D-tagatose, D-mannose, and isomaltulose, and even more preferably DFA III, nigerose, kojibiose, D-allulose, and D-tagatose, and most preferably DFA III, nigerose, kojibiose, D-allulose, D-tagatose, D-mannose, and isomaltulose, and most preferably DFA III, kojibiose, D-allulose.

In a preferred embodiment of the invention, the processed liquid nutrient is characterized by containing one or more altered carbohydrates wherein the altered carbohydrate is a disaccharide, selected from the group consisting of
for extracted fruit juice: nigerose, kojibiose, DFA III, cellobiose, trehalose, isomaltose, and isomaltulose, and preferably nigerose, kojibiose, DFA III, cellobiose, and isomaltulose, and more preferably nigerose, kojibiose, cellobiose, and isomaltulose, and even most preferably nigerose, and kojibiose; and/or
for a food preparation: DFA III, nigerose, kojibiose, isomaltose, isomaltulose, cellobiose, and trehalose, and preferably DFA III, nigerose, kojibiose, isomaltulose, and cellobiose, and more preferably DFA III, nigerose, kojibiose, and isomaltulose, and even more preferably DFA III, nigerose, and kojibiose, and most preferably DFA III and kojibiose.

In a preferred embodiment of the invention, the processed liquid nutrient is liquid milk and is characterized by containing one or more altered carbohydrates wherein the altered carbohydrate is D-allulose, D-mannose, galactose, glucose, fructose, and/or D-tagatose and is obtained from an initial carbohydrate at a conversion rate of 5 to 100%, of 5 to 95%, of 5 to 90%, of 5 to 85%, of 5 to 80%, of 5 to 75%, of 5 to 70%, of 5 to 65%, of 5 to 60%, of 5 to 55%, of 5 to 50%, of 5 to 45%, of 5 to 40%, of 5 to 35%, of 5 to 30%, of 5 to 25%, or of 5 to 20%, and preferably at a conversion rate of 10 to 100%, of 10 to 95%, of 10 to 90%, of 10 to 85%, of 10 to 80%, of 10 to 75%, of 10 to 70%, of 10 to 65%, of 10 to 60%, of 10 to 55%, of 10 to 50%, of 10 to 45%, of 10 to 40%, of 10 to 35%, of 10 to 30%, of 10 to 25%, or of 10 to 20%, and more preferably at a conversion rate of 15 to 100%, of 15 to 95%, of 15 to 90%, of 15 to 85%, of 15 to 80%, of 15 to 75%, of 15 to 70%, of 15 to 65%, of 15 to 60%, of 15 to 55%, of 15 to 50%, of 15 to 45%, of 15 to 40%, of 15 to 35%, of 15 to 30%, of 15 to 25%, or of 15 to 20%.

In a preferred embodiment of the invention, the processed liquid nutrient is extracted fruit juice and is characterized by containing one or more altered carbohydrates wherein the altered carbohydrate is nigerose, kojibiose, D-allulose, cellobiose, trehalose, isomaltulose, IMOs, GlucOS, isomaltose, DFA III, D-mannose, galactose, fructose, and/or glucose and is obtained from an initial carbohydrate at a conversion rate of 5 to 100%, of 5 to 95%, of 5 to 90%, of 5 to 85%, of 5 to 80%, of 5 to 75%, of 5 to 70%, of 5 to 65%, of 5 to 60%, of 5 to 55%, of 5 to 50%, of 5 to 45%, of 5 to 40%, of 5 to 35%, of 5 to 30%, of 5 to 25%, or of 5 to 20%, and preferably at a conversion rate of 10 to 100%, of 10 to 95%, of 10 to 90%, of 10 to 85%, of 10 to 80%, of 10 to 75%, of 10 to 70%, of 10 to 65%, of 10 to 60%, of 10 to 55%, of 10 to 50%, of 10 to 45%, of 10 to 40%, of 10 to 35%, of 10 to 30%, of 10 to 25%, or of 10 to 20%, and more preferably at a conversion rate of 15 to 100%, of 15 to 95%, of 15 to 90%, of 15 to 85%, of 15 to 80%, of 15 to 75%, of 15 to 70%, of 15 to 65%, of 15 to 60%, of 15 to 55%, of 15 to 50%, of 15 to 45%, of 15 to 40%, of 15 to 35%, of 15 to 30%, of 15 to 25%, or of 15 to 20%.

In a preferred embodiment of the invention, the processed liquid nutrient is a food preparation and is characterized by containing one or more altered carbohydrates wherein the altered carbohydrate is nigerose, kojibiose, D-allulose, cellobiose, trehalose, isomaltulose, IMOs, GlucOS, isomaltose, DFA III, D-mannose, D-tagatose, galactose, fructose, and/or glucose and is obtained from an initial carbohydrate at a conversion rate of 5 to 100%, of 5 to 95%, of 5 to 90%, of 5 to 85%, of 5 to 80%, of 5 to 75%, of 5 to 70%, of 5 to 65%, of 5 to 60%, of 5 to 55%, of 5 to 50%, of 5 to 45%, of 5 to 40%, of 5 to 35%, of 5 to 30%, of 5 to 25%, or of 5 to 20%, and preferably at a conversion rate of 10 to 100%, of 10 to 95%, of 10 to 90%, of 10 to 85%, of 10 to 80%, of 10 to 75%, of 10 to 70%, of 10 to 65%, of 10 to 60%, of 10 to 55%, of 10 to 50%, of 10 to 45%, of 10 to 40%, of 10 to 35%, of 10 to 30%, of 10 to 25%, or of 10 to 20%, and more preferably at a conversion rate of 15 to 100%, of 15 to 95%, of 15 to 90%, of 15 to 85%, of 15 to 80%, of 15 to 75%, of 15 to 70%, of 15 to 65%, of 15 to 60%, of 15 to 55%, of 15 to 50%, of 15 to 45%, of 15 to 40%, of 15 to 35%, of 15 to 30%, of 15 to 25%, or of 15 to 20%.

Preferably, the processed liquid nutrient contains the one or more altered carbohydrates in a concentration of at least, 0.01 wt.-%, or al least 0.03 wt.-%, or al least 0.05 wt.-%, or al least 0.08 wt.-%, or at least 0.1 wt.-%, or al least 0.3 wt.-%, or at least 0.5 wt.-%, or al least 0.8 wt.-%, or at least 1.0 wt.-%, or al least 3.0 wt.-%, or at least 5.0 wt.-%, in each case based on the total weight of all altered carbohydrates and relative to the total weight of the processed liquid nutrient.

In a preferred embodiment of the invention, the processed liquid nutrient is liquid milk and is characterized by containing one or more altered carbohydrates selected from the group consisting of
D-allulose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.5 wt.-%, of 0.1 to 0.3 wt.-% of 0.1 to 0.1 wt.-%; and/or
D-mannose in a concentration of of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.5 wt.-%, of 0.1 to 0.3 wt.-% of 0.1 to 0.1 wt.-%; and/or
D-tagatose in a concentration of from 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%;
   in each case relative to the total weight of the liquid milk.

In a preferred embodiment of the invention, the processed liquid nutrient is a non-concentrated, extracted fruit juice and is characterized by containing one or more altered carbohydrates selected from the group consisting of
D-allulose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2.5 wt.-%, of 0.01 to 2 wt.-%, of 0.01 to 1.5 wt.-%, 0.01 to 1.3 wt.-%, of 0.01 to 1 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2.3 wt.-%, of 0.05 to 2 wt.-%, of 0.05 to 1.5 wt.-%, of 0.05 to 1. wt.-%, of 0.05 to 1 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2.3 wt.-%, of 0.1 to 2 wt.-%, of 0.1 to 1.5 wt.-%, of 0.1 to 1.3 wt.-%, of 0.1 to 1 wt.-%, of 0.1 to 0.5 wt.-%, of 0.1 to 0.3 wt.-% of 0.1 to 0.1 wt.-%; and/or
D-mannose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-% of 0.1 to 0.1 wt.-%; and/or
D-tagatose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%; and/or
nigerose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.6 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.6 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.6 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%; and/or
kojibiose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.6 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.6 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.6 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%; and/or
IMO in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2.5 wt.-%, of 0.01 to 2 wt.-%, of 0.01 to 1.5 wt.-%, 0.01 to 1.3 wt.-%, of 0.01 to 1 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2.3 wt.-%, of 0.05 to 2 wt.-%, of 0.05 to 1.5 wt.-%, of 0.05 to 1. wt.-%, of 0.05 to 1 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2.3 wt.-%, of 0.1 to 2 wt.-%, of 0.1 to 1.5 wt.-%, of 0.1 to 1.3 wt.-%, of 0.1 to 1 wt.-%, of 0.1 to 0.5 wt.-%, of 0.1 to 0.3 wt.-% of 0.1 to 0.1 wt.-%; and/or
GlucOS in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2.5 wt.-%, of 0.01 to 2 wt.-%, of 0.01 to 1.5 wt.-%, 0.01 to 1.3 wt.-%, of 0.01 to 1 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2.3 wt.-%, of 0.05 to 2 wt.-%, of 0.05 to 1.5 wt.-%, of 0.05 to 1. wt.-%, of 0.05 to 1 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2.3 wt.-%, of 0.1 to 2 wt.-%, of 0.1 to 1.5 wt.-%, of 0.1 to 1.3 wt.-%, of 0.1 to 1 wt.-%, of 0.1 to 0.5 wt.-%, of 0.1 to 0.3 wt.-% of 0.1 to 0.1 wt.-%; and/or
Isomaltose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2.5 wt.-%, of 0.01 to 2 wt.-%, of 0.01 to 1.5 wt.-%, 0.01 to 1.3 wt.-%, of 0.01 to 1 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2.3 wt.-%, of 0.05 to 2 wt.-%, of 0.05 to 1.5 wt.-%, of 0.05 to 1. wt.-%, of 0.05 to 1 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2.3 wt.-%, of 0.1 to 2 wt.-%, of 0.1 to 1.5 wt.-%, of 0.1 to 1.3 wt.-%, of 0.1 to 1 wt.-%, of 0.1 to 0.5 wt.-%, of 0.1 to 0.3 wt.-% of 0.1 to 0.1 wt.-%; and/or
trehalose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.6 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.6 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.6 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%; and/or
cellobiose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.6 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.6 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.6 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%; and/or
isomaltulose in a concentration of 0.01 to 20 wt.-%, of 0.01 to 18 wt.-%, of 0.01 to 16 wt.-%, of 0.01 to 14 wt.-%, of 0.01 to 12 wt.-%, of 0.01 to 11 wt.-%, of 0.01 to 10 wt.-%, of 0.01 to 9 wt.-%, of 0.01 to 8 wt.-%, of 0.01 to 7 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.6 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from of 0.05 to 20 wt.-%, of 0.05 to 18 wt.-%, of 0.05 to 16 wt.-%, of 0.05 to 14 wt.-%, of 0.05 to 12 wt.-%, of 0.05 to 11 wt.-%, of 0.05 to 10 wt.-%, of 0.05 to 9 wt.-%, of 0.05 to 8 wt.-%, of 0.05 to 7 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.6 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably of 0.1 to 20 wt.-%, of 0.1 to 18 wt.-%, of 0.1 to 16 wt.-%, of 0.1 to 14 wt.-%, of 0.1 to 12 wt.-%, of 0.1 to 11 wt.-%, of 0.1 to 10 wt.-%, of 0.1 to 9 wt.-%, of 0.1 to 8 wt.-%, of 0.1 to 7 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.6 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.1 wt.-%; and/or
DFA III in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.6 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.6 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.6 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%;
   in each case relative to the total weight of the non-concentrated, extracted fruit juice.

In a preferred embodiment of the invention, the processed liquid nutrient is a concentrated extracted fruit juice and is characterized by containing one or more altered carbohydrates selected from the group consisting of
D-allulose in a concentration of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or
D-mannose in a of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or
D-tagatose in a concentration of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or
nigerose in a concentration of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or
kojibiose in a of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or
IMO in a of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or
GlucOS in a of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or
isomaltose in a of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or
trehalose in a concentration of of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or
cellobiose in a concentration of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or
isomaltulose in a concentration of 0.05 to 90 wt.-%, 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or
DFA III in a concentration of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%;
   in each case relative to the total weight of the concentrated extracted fruit juice.

In a preferred embodiment of the invention, the processed liquid nutrient is a food preparation and is characterized by containing one or more altered carbohydrates selected from the group consisting of
D-allulose in a concentration of 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or
D-mannose in a concentration of 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or
D-tagatose in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or
Nigerose in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or
Kojibiose in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or
IMO in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or
GlucOS in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or
isomaltose in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or
Trehalose in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or
Cellobiose in a 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or
Isomaltulose in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or
DFA III in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%;
   in each case relative to the total weight of the food preparation.

It is within the scope of this invention that any and all embodiments of the first aspect are also embodiments of any other embodiment of the invention, and that any and all embodiments of the second aspect are also embodiments of any other embodiment of the second aspect.

It is still a common belief that the use of enzymes is expensive. However, a number of applications have proven the opposite. Given the enzyme amounts used according to this invention someone skilled in the art of enzymology and enzyme production may recognize the economic feasibility of the invention. In the context of the present invention the term “carbohydrate” encompasses both small carbohydrates, such as monosaccharides and disaccharides, and larger saccharides, such as polysaccharides and oligosaccharides. In the context of the present invention, when a composition is said to comprise, contain or have a certain percentage of X wt.-% of a specified component, the weight percentage of the specified component is calculated relative to the total weight of the composition unless it is stated otherwise.

Particularly preferred embodiments 1 to 57 of the invention are summarized hereinafter: Embodiment 1: A method for the enzymatic processing of a virgin liquid nutrient comprising one or more initial carbohydrates into a processed liquid nutrient, the method comprising the steps of (i) providing a virgin liquid nutrient which comprises at least one initial carbohydrate, (ii) optionally adjusting pH value and/or temperature of the virgin liquid nutrient, (iii) optionally supplementing inorganic phosphate to the virgin liquid nutrient, and (iv) treating the virgin liquid nutrient with one or more enzymes, thereby converting at least a portion of the at least one initial carbohydrate into one or more altered carbohydrates and thus obtaining the processed liquid nutrient, wherein the processed liquid nutrient is preferably characterized by—a glycemic index of all initial carbohydrates and altered carbohydrates contained in the processed liquid nutrient, which is lower than the glycemic index of all initial carbohydrates contained in the virgin liquid nutrient; and/or—a caloric value of all initial carbohydrates and altered carbohydrates contained in the processed liquid nutrient, which is lower than the caloric value of all initial carbohydrates contained in the virgin liquid nutrient; and/or—a textural sensation conferred by all initial carbohydrates and altered carbohydrates contained in the processed liquid nutrient, which is connatural compared to the textural sensation conferred by all initial carbohydrates contained in the virgin liquid nutrient; and/or—a sweetness conferred by all initial carbohydrates and altered carbohydrates contained in the processed liquid nutrient, which is connatural compared to the sweetness conferred by all initial carbohydrates contained in the virgin liquid nutrient; wherein the method is preferably for preparing an edible processed liquid nutrient by enzymatic in-situ conversion of a virgin liquid nutrient, the method comprising the steps of (i) providing a virgin liquid nutrient which comprises one or more initial carbohydrates selected from the group consisting of sucrose, inulin, lactose, glucose, galactose, and fructose; (ii) optionally, adjusting pH value and/or temperature of the virgin liquid nutrient; (iii) optionally, supplementing inorganic phosphate to the virgin liquid nutrient; and (iv) treating the virgin liquid nutrient with one or more enzymes, thereby converting at least a portion of the one or more initial carbohydrates into one or more altered carbohydrates selected from the group consisting of kojibiose, nigerose, trehalose, cellobiose, alpha-D-fructofuranose beta-D-fructofuranose 1,2′:2,3′-dianhydride (DFA III), D-allulose, D-tagatose, isomaltulose, and D-mannose; thus obtaining the processed liquid nutrient. Embodiment 2: The method of embodiment 1, wherein the textural sensation conferred by all initial carbohydrates and altered carbohydrates contained in the processed liquid nutrient and the textural sensation conferred by all initial carbohydrates contained in the virgin liquid nutrient are expressed as—the viscosity or viscoelasticity conferred by all the carbohydrates, wherein the viscosity or viscoelasticity conferred by all initial carbohydrates and altered carbohydrates of the processed liquid nutrient in comparison to the viscosity or viscoelasticity conferred by all initial carbohydrates contained in the virgin liquid nutrient is connatural; and/or—the crystallinity conferred by all the carbohydrates, wherein the crystallinity conferred by all initial carbohydrates and altered carbohydrates of the processed liquid nutrient in comparison to the crystallinity conferred by all initial carbohydrates contained in the virgin liquid nutrient is connatural. Embodiment 3: The method of any previous embodiments, wherein the at least one altered carbohydrates are selected from the group consisting of monosaccharides and/or disaccharides. Embodiment 4: The method of any previous embodiments, wherein the at least one altered carbohydrates are disaccharides. Embodiment 5: The method of any previous embodiments, wherein the at least one altered carbohydrates are natural carbohydrates. Embodiment 6: The method of any of the preceding embodiments, wherein the at least one initial carbohydrates are selected from the group consisting of monosaccharides, disaccharides, oligosaccharides and/or polysaccharides. Embodiment 7: The method of any of the proceeding embodiments, wherein the virgin liquid nutrient is selected from the group consisting of—liquid milk; and/or—extracted fruit juice; and/or—a food preparation. Embodiment 8: The method of any of the preceding embodiments, wherein the at least one initial carbohydrate is selected from the group consisting of—for liquid milk: lactose, galactose, and glucose; and/or—for extracted fruit juice: sucrose, Inulin, glucose, and fructose; and/or—for a food preparation: lactose, sucrose, Inulin, glucose, galactose and fructose. Embodiment 9: The method of any of the preceding embodiments, wherein the at least one altered carbohydrate is selected from the group consisting of—for liquid milk: D-allulose, D-mannose, galactose, glucose, fructose, and D-tagatose; and preferably D-allulose, D-mannose, galactose, glucose, and D-tagatose; and more preferably D-allulose, D-mannose, and D-tagatose; and even more preferably D-allulose, and D-tagatose; and most preferably D-allulose; and/or—for extracted fruit juice: nigerose, kojibiose, D-allulose, D-mannose, glucose, fructose cellobiose, trehalose, isomaltulose, and DFA III; and preferably nigerose, kojibiose, D-allulose, D-mannose, glucose, fructose cellobiose, and DFA III; and more preferably nigerose, kojibiose, D-allulose, D-mannose, cellobiose, and DFA III; and most preferably nigerose, kojibiose, and D-allulose; and/or—for a food preparation: DFA III, nigerose, kojibiose, D-allulose, D-tagatose, D-mannose, isomaltulose, cellobiose, trehalose, galactose, glucose, and fructose; and preferably DFA III, nigerose, kojibiose, D-allulose, D-tagatose, D-mannose, isomaltulose, cellobiose, and trehalose; and more preferably DFA III, nigerose, kojibiose, D-allulose, D-tagatose, D-mannose, and isomaltulose; and even more preferably DFA III, nigerose, kojibiose, D-allulose, and D-tagatose; and most preferably DFA III, nigerose, kojibiose, D-allulose, D-tagatose, D-mannose, and isomaltulose, and most preferably DFA III, kojibiose, D-allulose. Embodiment 10: The method of any of the preceding embodiments, wherein the at least one altered carbohydrate is a disaccharide, selected from the group consisting of—for extracted fruit juice: nigerose, kojibiose, DFA III, cellobiose, trehalose, and isomaltulose; and preferably nigerose, kojibiose, DFA III, cellobiose, and isomaltulose; and more preferably nigerose, kojibiose, cellobiose, and isomaltulose; and most preferably nigerose, and kojibiose; and/or—for a food preparation: DFA III, nigerose, kojibiose, isomaltulose, cellobiose, and trehalose; and preferably DFA III, nigerose, kojibiose, isomaltulose, and cellobiose; and more preferably DFA III, nigerose, kojibiose, and isomaltulose; and even more preferably DFA III, nigerose, and kojibiose; and most preferably DFA III and kojibiose. Embodiment 11: The method of any of the preceding embodiments, wherein the enzyme-treated virgin liquid nutrient is characterized by—by a reduced glycemic index of at least 5% up to 100%; and/or—a reduced calorie count of at least 5% up to 100%; and/or—in a comparable textural sensation, and preferably in an identical textural sensation; and/or—in a comparable viscosity or viscoelasticity conferred by the carbohydrates, and preferably in an identical viscosity or viscoelasticity conferred by the carbohydrates; and/or—in a comparable crystallinity conferred by the carbohydrates, and preferably in an identical crystallinity conferred by the carbohydrates each and all in comparison to the virgin liquid nutrient. Embodiment 12: The method of any of the preceding embodiments, wherein the enzyme-treated, processed liquid nutrient is characterized by—a glycemic index which is reduced by at least 5% up to 100%, at least 10% up to 100%, at least 15% up to 100%, at least 20% up to 100%, at least 25% up to 100%, at least 30% up to 100%, at least 35% up to 100%, at least 40% up to 100%, at least 45% up to 100%, at least 50% up to 100%, at least 55% up to 100%, at least 60% up to 100%, at least 65% up to 100%, at least 70% up to 100%, at least 75% up to 100%, at least 80% up to 100%, or reduced by at least 5% up to 90%, at least 10% up to 90%, at least 15% up to 90%, at least 20% up to 90%, at least 25% up to 90%, at least 30% up to 90%, at least 35% up to 90%, at least 40% up to 90%, at least 45% up to 90%, at least 50% up to 90%, at least 55% up to 90%, at least 60% up to 90%, at least 65% up to 90%, at least 70% up to 90% or reduced by at least 5% up to 80%, at least 10% up to 80%, at least 15% up to 80%, at least 20% up to 80%, at least 25% up to 80%, at least 30% up to 80%, at least 35% up to 80%, at least 40% up to 80%, at least 45% up to 80%, at least 50% up to 80%, at least 55% up to 80%, at least 60% up to 80%, or reduced by at least 5% up to 70%, at least 10% up to 70%, at least 15% up to 70%, at least 20% up to 70%, at least 25% up to 70%, at least 30% up to 70%, at least 35% up to 70%, at least 40% up to 70%, at least 45% up to 70%, at least 50% up to 70%; and/or—a calorie count which is reduced by at least 5% up to 100%, at least 10% up to 100%, at least 15% up to 100%, at least 20% up to 100%, at least 25% up to 100%, at least 30% up to 100%, at least 35% up to 100%, at least 40% up to 100%, at least 45% up to 100%, at least 50% up to 100%, at least 55% up to 100%, at least 60% up to 100%, at least 65% up to 100%, at least 70% up to 100%, at least 75% up to 100%, at least 80% up to 100%, or reduced by at least 5% up to 90%, at least 10% up to 90%, at least 15% up to 90%, at least 20% up to 90%, at least 25% up to 90%, at least 30% up to 90%, at least 35% up to 90%, at least 40% up to 90%, at least 45% up to 90%, at least 50% up to 90%, at least 55% up to 90%, at least 60% up to 90%, at least 65% up to 90%, at least 70% up to 90 or reduced by at least 5% up to 80%, at least 10% up to 80%, at least 15% up to 80%, at least 20% up to 80%, at least 25% up to 80%, at least 30% up to 80%, at least 35% up to 80%, at least 40% up to 80%, at least 45% up to 80%, at least 50% up to 80%, at least 55% up to 80%, at least 60% up to 80%, or reduced by at least 5% up to 70%, at least 10% up to 70%, at least 15% up to 70%, at least 20% up to 70%, at least 25% up to 70%, at least 30% up to 70%, at least 35% up to 70%, at least 40% up to 70%, at least 45% up to 70%, at least 50% up to 70%;—in a comparable textural sensation, and preferably in an identical textural sensation; and/or—in a comparable viscosity or viscoelasticity conferred by the carbohydrates, and preferably in an identical viscosity or viscoelasticity conferred by the carbohydrates; and/or—in a comparable crystallinity conferred by the carbohydrates, and preferably in an identical crystallinity conferred by the carbohydrates each and all in comparison to the virgin liquid nutrient. Embodiment 13: The method according to any one of the preceding embodiments, wherein the at least one altered carbohydrate is characterized by a at least one, preferably two properties selected from the group consisting of—a glycemic index of from 0% up to 72%, from 0% up to 68%, from 0% up to 60%, from 0% up to 55%, from 0% up to 50%, from 0% up to 45%, from 0% up to 40%, from 0% up to 35%, from 0% up to 32%, from 0% up to 30%, from 0% up to 25%, from 0% up to 20%, from 0% up to 19%, from 0% up to 15%, from 0% up to 10%, from 0% up to 5%, from 0% up to 3%, or from 0% up to 72%, from 3% up to 68%, from 3% up to 60%, from 3% up to 55%, from 3% up to 50%, from 3% up to 45%, from 3% up to 40%, from 3% up to 35%, from 3% up to 32%, from 3% up to 30%, from 3% up to 25%, from 3% up to 20%, from 3% up to 19%, from 3% up to 15%, from 3% up to 10%, from 3% up to 5%, and preferably of from 0% up to 15%, from 0% up to 10%, from 0% up to 5%, from 0% up to 3%, or from 3% up to 15%, from 3% up to 10%, from 3% up to 5%, and most preferably of below 10%; and/or—a calorie count of from 0 kcal/g up 4 kcal/g, from 0 kcal/g up 3.9 kcal/g, from 0 kcal/g up 3.5 kcal/g, from 0 kcal/g up 3 kcal/g, from 0 kcal/g up 2.5 kcal/g, from 0 kcal/g up 2 kcal/g, from 0 kcal/g up 1.7 kcal/g, from 0 kcal/g up 1.5 kcal/g, from 0 kcal/g up 0.3 kcal/g, or from 0.2 kcal/g up 4 kcal/g, from 0.2 kcal/g up 3.9 kcal/g, from 0.2 kcal/g up 3.5 kcal/g, from 0.2 kcal/g up 3 kcal/g, from 0.2 kcal/g up 2.5 kcal/g, from 0.2 kcal/g up 2 kcal/g, from 0.2 kcal/g up 1.7 kcal/g, from 0.2 kcal/g up 1.5 kcal/g, from 0.2 kcal/g up 0.3 kcal/g. Embodiment 14: The method of any of the preceding embodiments, wherein the at least one altered carbohydrate is characterized by the following combinations of properties:—a glycemic index of from 0% up to 15%, from 0% up to 10%, from 0% up to 5%, or from 3% up to 15%, from 3% up to 10%, from 3% up to 5%, and most preferably of from 0% up to 5%; and—a calorie count of from 0 kcal/g up 2 kcal/g, from 0 kcal/g up 1.7 kcal/g, from 0 kcal/g up 1.5 kcal/g, from 0 kcal/g up 0.3 kcal/g, or from 0.2 kcal/g up 2 kcal/g, from 0.2 kcal/g up 1.7 kcal/g, from 0.2 kcal/g up 1.5 kcal/g, from 0.2 kcal/g up 0.3 kcal/g. Embodiment 15: The method according to any one of the preceding embodiments, wherein the at least one altered carbohydrate is selected from the group consisting of D-allulose, D-tagatose, nigerose, kojibiose, cellobiose, and/or DFA III. Embodiment 16: The method of any of the preceding embodiments, wherein in step (iv) the treatment of the virgin liquid nutrient into a processed liquid nutrient with the one or more enzymes occurs (i) in a one-step process upon simultaneous adding of the one or more enzymes and without interim purification of the partially processed liquid nutrient intermediate; or (ii) in a one-step process upon sequential adding of the one or more enzymes and without interim purification of the partially processed liquid nutrient intermediate; or (iii) in a multi-step process upon sequential adding of the one or more enzymes and with interim purification of the partially processed liquid nutrient intermediate. Embodiment 17: The method of any of the preceding embodiments, wherein method is characterized by the adjustment of the pH value of the virgin liquid nutrient in step (ii), and wherein preferably, the pH value is adjusted to any pH value selected from the group consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, or 9.9; and more preferably selected from the group consisting of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, and even more preferably selected from the group consisting of pH 5.0 to 7.5, pH 3.0 to pH 6.0, pH 4.0 to 7.0, pH 3.5 to 6.5, pH 4.0 to 6.5, and pH 4.5 to 6.5. Embodiment 18: The method of any of the preceding embodiments, wherein the method is characterized by the supplementing inorganic phosphate to the virgin liquid nutrient in step (iii). Preferably, inorganic phosphate is supplemented to a final concentration in the virgin liquid nutrient of from 1 mM and 500 mM, from 1 mM and 450 mM, from 1 mM and 400 mM, from 1 mM and 350 mM, from 1 mM and 300 mM, from 1 mM and 250 mM, from 1 mM and 200 mM, from 1 mM and 150 mM, and preferably from 10 mM and 150 mM. Embodiment 19: The method of any of the preceding embodiments, wherein the method is characterized by the supplementing of inorganic phosphate for the formation of the altered carbohydrates trehalose and/or cellobiose. Embodiment 20: The method of any of the preceding embodiments, wherein the method is characterized in that in step (iv) the treating of the virgin liquid nutrient with one or more enzymes occurs at a temperature and for reaction times, which are required convert the virgin liquid nutrient into a processed liquid nutrient, an preferably at a temperature and for reaction times, which are required to reach or approach the thermodynamic equilibrium of the reaction. Embodiment 21: The method of any of the preceding embodiments, wherein the method is characterized in treating the virgin liquid nutrient with one or more enzymes in step (iv) by adding the one and more enzymes to the virgin liquid nutrient, which after conversion of the one or more initial carbohydrates into one or more altered carbohydrates remain part of the processed liquid nutrient and the foodstuff product derived therefrom; and/or—by adding the one and more enzymes to the virgin liquid nutrient, which after conversion of the one or more initial carbohydrates into one or more altered carbohydrates are removed from the processed liquid nutrient or from the foodstuff product derived therefrom; and/or—by adding the one and more enzymes in an immobilized formulation to the virgin liquid nutrient, which after conversion of the one or more initial carbohydrates into one or more altered carbohydrates is removed from the processed liquid nutrient and the foodstuff product derived therefrom by means of column separation; and/or—by contacting the one and more enzymes in an immobilized formulation with the virgin liquid nutrient, for example by column technologies, wherein after conversion of the one or more initial carbohydrates into one or more altered carbohydrates the processed liquid nutrient and the foodstuff product derived therefrom are released eluted from the column. Embodiment 22: The method of any of the preceding embodiments, wherein the virgin liquid nutrient is treated with one enzyme catalyzing one conversions of initial carbohydrates into one or more altered carbohydrate selected from the group consisting of conversions—initial carbohydrate glucose into altered carbohydrate fructose; and/or—initial carbohydrate glucose into altered carbohydrate D-mannose; and/or—initial carbohydrate fructose into altered carbohydrate glucose; and/or—initial carbohydrate fructose into altered carbohydrate D-allulose; and/or—initial carbohydrate fructose into altered carbohydrate D-mannose; and/or—initial carbohydrate inulin into altered carbohydrate DFA; and/or—initial carbohydrate sucrose into altered carbohydrates fructose and glucose; and/or—initial carbohydrate sucrose into altered carbohydrate kojibiose; and/or—initial carbohydrate sucrose into altered carbohydrate nigerose; and/or—initial carbohydrate sucrose into altered carbohydrate glucose-1-phosphate; and/or—initial carbohydrate sucrose into altered carbohydrate isomaltulose; and/or—initial carbohydrate galactose into altered carbohydrate D-tagatose; and/or—initial carbohydrate lactose into altered carbohydrates galactose and glucose. Embodiment 23: The method of any of the preceding embodiments, wherein the virgin liquid nutrient is treated with a first enzyme catalyzing one conversion of initial carbohydrates into one or more first altered carbohydrates, and wherein the one or more first altered carbohydrates is concomitantly treated with one or more additional enzymes catalyzing one or more conversions into a second altered carbohydrate selected from the group consisting of conversions—first altered carbohydrate glucose into second altered carbohydrate D-fructose; and/or—first altered carbohydrate glucose into second altered carbohydrate D-mannose; and/or—first altered carbohydrate fructose into second altered carbohydrate glucose; and/or—first altered carbohydrate fructose into second altered carbohydrate D-allulose; and/or—first altered carbohydrate fructose into second altered carbohydrate D-mannose; and/or—first altered carbohydrate galactose into second altered carbohydrate D-tagatose; and/or—first altered carbohydrate glucose-1-phosphate into second altered carbohydrate cellobiose; and/or—first altered carbohydrate glucose-1-phosphate into second altered carbohydrate cellobiose. Embodiment 24: The method of embodiment 23, wherein the one or more first altered carbohydrates is subsequently treated with one or more additional enzymes catalyzing one or more conversions into a second altered carbohydrate selected from the group consisting of conversions of embodiment 22. Embodiment 25: The method of embodiment 24, wherein the first step of converting an initial carbohydrate into a first altered carbohydrate and the second step of converting a first altered carbohydrate into a second altered carbohydrate can be accomplished (i) in a one-step process upon simultaneous adding of the one or more enzymes for both steps without interim purification of the partially processed liquid nutrient intermediate; or (ii) in a one-step process upon sequential adding of the one or more enzymes for both steps and without interim purification of the partially processed liquid nutrient intermediate; or (iii) in a multi-step process upon sequential adding of the one or more enzymes for both steps with interim purification of the partially processed liquid nutrient intermediate. Embodiment 26: The method of any of the preceding embodiments, wherein the virgin liquid nutrient is treated with two enzymes catalyzing the conversion of one initial carbohydrate into two or more altered carbohydrates selected from the group consisting of conversions—initial carbohydrate fructose into altered carbohydrates glucose and D-allulose; and/or—initial carbohydrate fructose into altered carbohydrates glucose and D-mannose; and/or—initial carbohydrate fructose into altered carbohydrates D-allulose and D-mannose; and/or—initial carbohydrate lactose into altered carbohydrates galactose and glucose and D-tagatose; and/or—initial carbohydrate sucrose into altered carbohydrates cellobiose and fructose; and/or—initial carbohydrate sucrose into altered carbohydrates trehalose and fructose; and/or—initial carbohydrate sucrose into altered carbohydrates glucose and D-allulose and fructose; and/or—initial carbohydrate sucrose into altered carbohydrates glucose and D-mannose and fructose; and/or—initial carbohydrate sucrose into altered carbohydrates fructose and kojibiose; and/or—initial carbohydrate sucrose into altered carbohydrates fructose and nigerose. Embodiment 27: The method of any of the preceding embodiments, wherein the virgin liquid nutrient is treated with two enzymes catalyzing the conversion of two or more initial carbohydrates into two or altered carbohydrates selected from the group consisting of conversions—initial carbohydrates fructose and inulin into altered carbohydrates D-allulose and DFA; and/or—initial carbohydrates fructose and inulin into altered carbohydrates D-mannose and DFA; and/or—initial carbohydrates sucrose and inulin into altered carbohydrates isomaltulose and DFA; and/or—initial carbohydrates sucrose and inulin into altered carbohydrates kojibiose and DFA; and/or—initial carbohydrates sucrose and inulin into altered carbohydrates nigerose and DFA; and/or—initial carbohydrates sucrose and fructose into altered carbohydrates isomaltulose and D-allulose; and/or—initial carbohydrates sucrose and fructose into altered carbohydrates kojibiose and D-allulose; and/or—initial carbohydrates sucrose and fructose into altered carbohydrates nigerose and D-allulose; and/or—initial carbohydrates sucrose and fructose into altered carbohydrates isomaltulose and D-mannose; and/or—initial carbohydrates sucrose and fructose into altered carbohydrates kojibiose and D-mannose; and/or—initial carbohydrates sucrose and fructose into altered carbohydrates nigerose and D-mannose; and/or—initial carbohydrates sucrose and glucose into altered carbohydrates isomaltulose and fructose; and/or—initial carbohydrates sucrose and glucose into altered carbohydrates kojibiose and fructose; and/or—initial carbohydrates sucrose and glucose into altered carbohydrates nigerose and fructose; and/or—initial carbohydrates lactose and glucose into altered carbohydrates galactose and D-tagatose; and/or—initial carbohydrates lactose and galactose into altered carbohydrates glucose and fructose; and/or—initial carbohydrates glucose and fructose and inulin into altered carbohydrates D-allulose and DFA; and/or—initial carbohydrates glucose and fructose and inulin into altered carbohydrates D-mannose and DFA; and/or—initial carbohydrates sucrose and fructose and inulin into altered carbohydrates D-allulose and DFA; and/or—initial carbohydrates sucrose and fructose and inulin into altered carbohydrates D-mannose and DFA; and/or—initial carbohydrates sucrose and fructose and inulin into altered carbohydrates isomaltulose and DFA; and/or—initial carbohydrates sucrose and fructose and inulin into altered carbohydrates kojibiose and DFA; and/or—initial carbohydrates sucrose and fructose and inulin into altered carbohydrates nigerose and DFA; and/or—initial carbohydrates sucrose and fructose and inulin into altered carbohydrates isomaltulose and D-allulose; and/or—initial carbohydrates sucrose and fructose and inulin into altered carbohydrates kojibiose and D-allulose; and/or—initial carbohydrates sucrose and fructose and inulin into altered carbohydrates nigerose and D-allulose; and/or—initial carbohydrates sucrose and fructose and inulin into altered carbohydrates isomaltulose and D-mannose; and/or—initial carbohydrates sucrose and fructose and inulin into altered carbohydrates kojibiose and D-mannose; and/or—initial carbohydrates sucrose and fructose and inulin into altered carbohydrates nigerose and D-mannose; and/or—initial carbohydrates sucrose and glucose and inulin into altered carbohydrates isomaltulose and DFA; and/or—initial carbohydrates sucrose and glucose and inulin into altered carbohydrates kojibiose and DFA; and/or—initial carbohydrates sucrose and glucose and inulin into altered carbohydrates nigerose and DFA; and/or—initial carbohydrates sucrose and fructose and glucose into altered carbohydrates isomaltulose and D-allulose; and/or—initial carbohydrates sucrose and fructose and glucose into altered carbohydrates kojibiose and D-allulose; and/or—initial carbohydrates sucrose and fructose and glucose into altered carbohydrates nigerose and D-allulose; and/or—initial carbohydrates sucrose and fructose and glucose into altered carbohydrates isomaltulose and D-mannose; and/or—initial carbohydrates sucrose and fructose and glucose into altered carbohydrates kojibiose and D-mannose; and/or—initial carbohydrates sucrose and fructose and glucose into altered carbohydrates nigerose and D-mannose. Embodiment 28: The method of any of the preceding embodiments, wherein the virgin liquid nutrient is treated with three and more enzymes catalyzing the conversion of one or more initial carbohydrates into one or more altered carbohydrates selected from the group consisting of conversions—initial carbohydrate sucrose into altered carbohydrates fructose, glucose, D-mannose and D-allulose; and/or—initial carbohydrate sucrose into altered carbohydrates cellobiose and glucose and fructose; and/or—initial carbohydrate sucrose into altered carbohydrates trehalose and glucose and fructose; and/or—initial carbohydrate sucrose into altered carbohydrates kojibiose and D-allulose; and/or—initial carbohydrate sucrose into altered carbohydrates kojibiose and D-mannose; and/or—initial carbohydrate sucrose into altered carbohydrates kojibiose and D-allulose and D-mannose; and/or—initial carbohydrate sucrose into altered carbohydrates nigerose and D-allulose; and/or—initial carbohydrate sucrose into altered carbohydrates nigerose and D-mannose; and/or—initial carbohydrate sucrose into altered carbohydrates nigerose and D-allulose and D-mannose; and/or—initial carbohydrate lactose into altered carbohydrates glucose and galactose and fructose and D-tagatose; and/or—initial carbohydrate lactose into altered carbohydrates glucose and galactose and fructose and D-tagatose and D-allulose; and/or—initial carbohydrate lactose into altered carbohydrates glucose and galactose and fructose and D-tagatose and D-mannose; and/or—initial carbohydrate lactose into altered carbohydrates glucose and galactose and fructose and D-tagatose and D-allulose and D-mannose; and/or—initial carbohydrate fructose into altered carbohydrates D-allulose and D-mannose; and/or—initial carbohydrate sucrose and fructose into altered carbohydrates glucose and D-allulose; and/or—initial carbohydrate sucrose and fructose into altered carbohydrates glucose and D-mannose; and/or—initial carbohydrate sucrose and fructose into altered carbohydrates glucose and D-allulose and D-mannose; and/or—initial carbohydrate sucrose and fructose into altered carbohydrates isomaltulose glucose and D-allulose; and/or—initial carbohydrate sucrose and fructose into altered carbohydrates isomaltulose glucose and D-mannose; and/or—initial carbohydrate sucrose and fructose into altered carbohydrates isomaltulose glucose and D-allulose and D-mannose; and/or—initial carbohydrate sucrose and fructose into altered carbohydrates cellobiose and glucose; and/or—initial carbohydrate sucrose and fructose into altered carbohydrates trehalose and glucose; and/or—initial carbohydrate sucrose and fructose into altered carbohydrates kojibiose and D-allulose; and/or—initial carbohydrate sucrose and fructose into altered carbohydrates kojibiose and D-mannose; and/or—initial carbohydrate sucrose and fructose into altered carbohydrates kojibiose and D-allulose and D-mannose; and/or—initial carbohydrate sucrose and fructose into altered carbohydrates nigerose and D-allulose; and/or—initial carbohydrate sucrose and fructose into altered carbohydrates nigerose and D-mannose; and/or—initial carbohydrate sucrose and fructose into altered carbohydrates nigerose and D-allulose and D-mannose; and/or—initial carbohydrate sucrose and glucose into altered carbohydrates fructose and D-allulose; and/or—initial carbohydrate sucrose and glucose into altered carbohydrates fructose and D-mannose; and/or—initial carbohydrate sucrose and glucose into altered carbohydrates fructose and D-allulose and D-mannose; and/or—initial carbohydrate sucrose and glucose into altered carbohydrates isomaltulose and fructose and D-allulose; and/or—initial carbohydrate sucrose and glucose into altered carbohydrates isomaltulose and fructose and D-mannose; and/or—initial carbohydrate sucrose and glucose into altered carbohydrates isomaltulose and fructose and D-allulose and D-mannose; and/or—initial carbohydrate sucrose and glucose into altered carbohydrates cellobiose and fructose; and/or—initial carbohydrate sucrose and glucose into altered carbohydrates trehalose and fructose; and/or—initial carbohydrate sucrose and glucose into altered carbohydrates kojibiose and D-allulose; and/or—initial carbohydrate sucrose and glucose into altered carbohydrates kojibiose and D-mannose; and/or—initial carbohydrate sucrose and glucose into altered carbohydrates kojibiose and D-allulose and D-mannose; and/or—initial carbohydrate sucrose and glucose into altered carbohydrates nigerose and D-allulose; and/or—initial carbohydrate sucrose and glucose into altered carbohydrates nigerose and D-mannose; and/or—initial carbohydrate sucrose and glucose into altered carbohydrates nigerose and D-allulose and D-mannose; and/or—initial carbohydrate sucrose and inulin into altered carbohydrates glucose and fructose and D-allulose and DFA; and/or—initial carbohydrate sucrose and inulin into altered carbohydrates glucose and fructose and D-mannose and DFA; and/or—initial carbohydrate sucrose and inulin into altered carbohydrates glucose and fructose and D-allulose and D-mannose and DFA; and/or—initial carbohydrate sucrose and inulin into altered carbohydrates isomaltulose and glucose and fructose and D-allulose and DFA; and/or—initial carbohydrate sucrose and inulin into altered carbohydrates isomaltulose and glucose and fructose and D-mannose and DFA; and/or—initial carbohydrate sucrose and inulin into altered carbohydrates isomaltulose and glucose and fructose and D-allulose and D-mannose and DFA; and/or—initial carbohydrate sucrose and inulin into altered carbohydrates cellobiose and glucose and fructose and DFA; and/or—initial carbohydrate sucrose and inulin into altered carbohydrates trehalose and glucose and fructose and DFA; and/or—initial carbohydrate sucrose and inulin into altered carbohydrates kojibiose and D-allulose and DFA; and/or—initial carbohydrate sucrose and inulin into altered carbohydrates kojibiose and D-mannose and DFA; and/or—initial carbohydrate sucrose and inulin into altered carbohydrates kojibiose and D-allulose and D-mannose and DFA; and/or—initial carbohydrate sucrose and inulin into altered carbohydrates nigerose and D-allulose and DFA; and/or—initial carbohydrate sucrose and inulin into altered carbohydrates nigerose and D-mannose and DFA; and/or—initial carbohydrate sucrose and inulin into altered carbohydrates nigerose and D-allulose and D-mannose and DFA; and/or—initial carbohydrate sucrose and inulin into altered carbohydrates kojibiose and fructose and DFA; and/or—initial carbohydrate sucrose and inulin into altered carbohydrates nigerose and fructose and DFA; and/or—initial carbohydrate sucrose and fructose and inulin into altered carbohydrates glucose and D-allulose and DFA; and/or—initial carbohydrate sucrose and fructose and inulin into altered carbohydrates glucose and D-mannose and DFA; and/or—initial carbohydrate sucrose and fructose and inulin into altered carbohydrates glucose and D-allulose and D-mannose and DFA; and/or—initial carbohydrate sucrose and fructose and inulin into altered carbohydrates isomaltulose and D-allulose and DFA; and/or—initial carbohydrate sucrose and fructose and inulin into altered carbohydrates isomaltulose and D-mannose and DFA; and/or—initial carbohydrate sucrose and fructose and inulin into altered carbohydrates isomaltulose and D-allulose and D-mannose and DFA; and/or—initial carbohydrate sucrose and fructose and inulin into altered carbohydrates cellobiose and glucose and DFA; and/or—initial carbohydrate sucrose and fructose and inulin into altered carbohydrates trehalose and glucose and DFA; and/or—initial carbohydrate sucrose and fructose and inulin into altered carbohydrates kojibiose and D-allulose and DFA; and/or—initial carbohydrate sucrose and fructose and inulin into altered carbohydrates kojibiose and D-mannose and DFA; and/or—initial carbohydrate sucrose and fructose and inulin into altered carbohydrates kojibiose and D-allulose and D-mannose and DFA; and/or—initial carbohydrate sucrose and fructose and inulin into altered carbohydrates nigerose and D-allulose and DFA; and/or—initial carbohydrate sucrose and fructose and inulin into altered carbohydrates nigerose and D-mannose and DFA; and/or—initial carbohydrate sucrose and fructose and inulin into altered carbohydrates nigerose and D-allulose and D-mannose and DFA; and/or—initial carbohydrate sucrose and fructose and inulin into altered carbohydrates kojibiose and glucose and DFA; and/or—initial carbohydrate sucrose and fructose and inulin into altered carbohydrates nigerose and glucose and DFA; and/or—initial carbohydrate sucrose and glucose and inulin into altered carbohydrates fructose and D-allulose and DFA; and/or—initial carbohydrate sucrose and glucose and inulin into altered carbohydrates fructose and D-mannose and DFA; and/or—initial carbohydrate sucrose and glucose and inulin into altered carbohydrates fructose and D-allulose and D-mannose and DFA; and/or—initial carbohydrate sucrose and glucose and inulin into altered carbohydrates isomaltulose and fructose and D-allulose and DFA; and/or—initial carbohydrate sucrose and glucose and inulin into altered carbohydrates cellobiose and fructose and DFA; and/or—initial carbohydrate sucrose and glucose and inulin into altered carbohydrates trehalose and fructose and DFA; and/or—initial carbohydrate sucrose and glucose and inulin into altered carbohydrates kojibiose and D-allulose and DFA; and/or—initial carbohydrate sucrose and glucose and inulin into altered carbohydrates kojibiose and D-mannose and DFA; and/or—initial carbohydrate sucrose and glucose and inulin into altered carbohydrates kojibiose and D-allulose and D-mannose and DFA; and/or—initial carbohydrate sucrose and glucose and inulin into altered carbohydrates nigerose and D-allulose and DFA; and/or—initial carbohydrate sucrose and glucose and inulin into altered carbohydrates nigerose and D-mannose and DFA; and/or—initial carbohydrate sucrose and glucose and inulin into altered carbohydrates nigerose and D-allulose and D-mannose and DFA; and/or—initial carbohydrate sucrose and glucose and inulin into altered carbohydrates kojibiose and fructose and DFA; and/or—initial carbohydrate sucrose and glucose and inulin into altered carbohydrates nigerose and fructose and DFA; and/or—initial carbohydrate lactose and galactose into altered carbohydrates glucose and fructose and D-tagatose; and/or—initial carbohydrate lactose and galactose into altered carbohydrates glucose and fructose and D-tagatose and D-allulose; and/or—initial carbohydrate lactose and galactose into altered carbohydrates glucose and fructose and D-tagatose and D-mannose; and/or—initial carbohydrate lactose and galactose into altered carbohydrates glucose and fructose and D-tagatose and D-allulose and D-mannose; and/or—initial carbohydrate lactose and glucose into altered carbohydrates galactose and fructose and D-tagatose; and/or—initial carbohydrate lactose and glucose into altered carbohydrates galactose and fructose and D-tagatose and D-allulose; and/or—initial carbohydrate lactose and glucose into altered carbohydrates galactose and fructose and D-tagatose and D-mannose; and/or—initial carbohydrate lactose and glucose into altered carbohydrates galactose and fructose and D-tagatose and D-allulose and D-mannose. Embodiment 29: The method of any of the preceding embodiments, wherein the enzymes for treatment of the virgin liquid nutrient in step (iv) are selected from the group consisting of—enzymes from EC classes EC 5.1.3.30, EC 5.3.1.4, EC 3.2.1.26, EC 5.3.1.5, EC 5.3.1.7, EC 3.2.1.23, EC 2.4.1.7, EC 2.4.1.64, EC 2.4.1.20, EC 5.1.3.11, and EC 4.2.2.18; and/or—enzymes with the name D-psicose-3-epimerase (EC 5.1.3.30), L-arabinose-isomerase (EC 5.3.1.4), invertase (or beta-fructofuranosidase, EC 3.2.1.26), glucose-isomerase (EC 5.3.1.5), mannose-isomerase (EC 5.3.1.7), beta-galactosidase (EC 3.2.1.23), sucrose phosphorylase (EC 2.4.1.7), trehalose phosphorylase (EC 2.4.1.64), cellobiose phosphorylase (EC 2.4.1.20), cellobiose-2-epimerase (EC 5.1.3.11), and inulin fructotransferase (EC 4.2.2.18) and for each group enclosing both, the wild-type enzymes as well as improved enzyme variants obtained by improved enzyme obtained by engineering. Embodiment 30: The method of any of the preceding embodiments, wherein virgin liquid nutrient in step (iv) is treated with one or more enzymes characterized by one or more functional features (A), (B), (C), (D), (E), (A) a catalytic activity for carbohydrate forming in the virgin liquid nutrient of at least 1 to 5000 enzyme units per 100 grams virgin liquid nutrient, at least 25 to 5000 enzyme units per 100 grams virgin liquid nutrient, and preferably about 100 to about 2000 units per 100 grams virgin liquid nutrient; (B) a high catalytic activity at the pH of the virgin liquid nutrient selected from the group consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, or 9.9; and more preferably selected from the group consisting of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, and even more preferably selected from the group consisting of pH 5.0 to 7.5, pH 3.0 to pH 6.0, pH 4.0 to 7.0, pH 3.5 to 6.5, pH 4.0 to 6.5, and pH 4.5 to 6.5; (C) a high process stability in the environment of the virgin liquid nutrient expressed at thermal stability for from 1 hour up to 672 hours, from 1 hour up to 500 hours, from 1 hour up to 400 hours, from 1 hour up to 300 hours, from 1 hour up to 200 hours, from 1 hour up to 168 hours, from 1 hour up to 144 hours, from 1 hour up to 120 hours, from 1 hour up to 96 hours, from 1 hour up to 72 hours, from 1 hour up to 48 hours, from 1 hour up to 24 hours, from 1 hour up to 12 hours, or from 1 hour up to 6 hours; (D) a high activity at high concentrations of one or more initial carbohydrates of from 0.5 to 70 wt.-%, from 0.5 to 65 wt.-%, from 0.5 to 60 wt.-%, from 0.5 to 55 wt.-%, from 0.5 to 50 wt.-%, from 0.5 to 45 wt.-%, from 0.5 to 40 wt.-%, from 0.5 to 35 wt.-%, from 0.5 to 30 wt.-%, from 0.5 to 25 wt.-%, from 0.5 to 20 wt.-%, or from 0.5 to 15 wt.-%; or from 1 to 70 wt.-%, from 1 to 65 wt.-%, from 1 to 60 wt.-%, from 1 to 55 wt.-%, from 1 to 50 wt.-%, from 1 to 45 wt.-%, from 1 to 40 wt.-%, from 1 to 35 wt.-%, from 1 to 30 wt.-%, from 1 to 25 wt.-%, from 1 to 20 wt.-%, or from 1 to 15 wt.-%; or from 3 to 70 wt.-%, from 3 to 65 wt.-%, from 3 to 60 wt.-%, from 3 to 55 wt.-%, from 3 to 50 wt.-%, from 3 to 45 wt.-%, from 3 to 40 wt.-%, from 3 to 35 wt.-%, from 3 to 30 wt.-%, from 3 to 25 wt.-%, from 3 to 20 wt.-%, or from 3 to 15 wt.-%; (E) a high activity at high concentrations of one or more altered carbohydrates of from 5 to 70 wt.-%, from 5 to 65 wt.-%, from 5 to 60 wt.-%, from 5 to 55 wt.-%, from 5 to 50 wt.-%, from 5 to 45 wt.-%, from 5 to 40 wt.-%, from 5 to 35 wt.-%, from 5 to 30 wt.-%, from 5 to 25 wt.-%, from 5 to 20 wt.-%, or from 5 to 15 wt.-%; or from 10 to 70 wt.-%, from 10 to 65 wt.-%, from 10 to 60 wt.-%, from 10 to 55 wt.-%, from 10 to 50 wt.-%, from 10 to 45 wt.-%, from 10 to 40 wt.-%, from 10 to 35 wt.-%, from 10 to 30 wt.-%, from 10 to 25 wt.-%, from 10 to 20 wt.-%, or from 10 to 15 wt.-%; or from 15 to 70 wt.-%, from 15 to 65 wt.-%, from 15 to 60 wt.-%, from 15 to 55 wt.-%, from 15 to 50 wt.-%, from 15 to 45 wt.-%, from 15 to 40 wt.-%, from 15 to 35 wt.-%, from 15 to 30 wt.-%, from 15 to 25 wt.-%, or from 15 to 20 wt.-%. Embodiment 31: The method of any of the preceding embodiments, wherein liquid milk in step (iv) is treated with one or more enzymes characterized by one or more functional features (A), (B), (C), (D), (A) high catalytic activity in the environment of the liquid milk, namely being active in biphasic milk emulsion; (B) high catalytic activity at the pH of the liquid milk, namely at pH from 4.0 to 8.5, from 4.5 to 8.0, from 5.0 to 8.0, and preferably from pH 5.0 to 7.5; (C) high process stability in the environment of the liquid milk, namely being stable in biphasic milk emulsions; (D) high activity at low concentrations of one or more initial carbohydrates, namely at lactose concentration of at least 3.0 wt.-%, and glucose and/or galactose concentrations of at least 1.0 wt.-% each. Embodiment 32: The method of any of the preceding embodiments, wherein extracted fruit juice in step (iv) is treated with one or more enzymes characterized by one or more functional features (A), (B), (C), (D), (E), (A) high catalytic activity in the environment of the extracted fruit juice; (B) high catalytic activity at the pH of the extracted fruit juice, namely from pH 1.0 to pH 8.0, from pH 2.0 to pH 7.0, from pH 2.5 to pH 7.5, and preferably from pH 3.0 to pH 6.0; (C) high process stability in the environment of the extracted fruit juice; (D) high activity at low concentrations of one or more initial carbohydrates, namely sucrose, glucose, fructose concentration of at least 1.0 wt.-%; (E) high activity at high concentrations of one or more altered carbohydrates, namely at concentrations higher 10 wt.-% of sucrose, fructose, or glucose as they occur naturally or in concentrated extracted fruit juice. Embodiment 33: The method of any of the preceding embodiments, wherein a food preparation in step (iv) is treated with one or more enzymes characterized by one or more functional features (A), (B), (C), (D), (E), (A) high catalytic activity in the environment of the food preparation; (B) high catalytic activity at the pH of the food preparation, namely pH 4.0 to 7.0; (C) high process stability in the environment of the food preparation; (D) high activity at low concentrations of one or more initial carbohydrates, namely sucrose, glucose, fructose concentration of at least 1.0 wt.-%; (E) high activity at high concentrations of one or more altered carbohydrates, namely at concentrations higher 10 wt.-% of sucrose, fructose, glucose, and/or inulin as they occur naturally in ingredients used in the food preparation or added as such to the food preparation. Embodiment 34: The method of any of the preceding embodiments, wherein the virgin liquid nutrient in step (iv) is treated with improved enzymes of any one of embodiments 29 to 33. Embodiment 35: The method of any of the preceding embodiments, wherein the processed liquid nutrient is used as an ingredient for mixing with other food ingredients, further processing or confectioning, or for preparation of a food preparation. Embodiment 36: The method according to any of the preceding embodiments, wherein the processed liquid nutrient is preferably characterized by—a glycemic index of all initial carbohydrates and altered carbohydrates contained in the processed liquid nutrient, which is lower than the glycemic index of all initial carbohydrates contained in the virgin liquid nutrient; and/or—a calorie count of all initial carbohydrates and altered carbohydrates contained in the processed liquid nutrient, which is lower than the calorie count of all initial carbohydrates contained in the virgin liquid nutrient; and/or—the textural sensation conferred by all initial carbohydrates and altered carbohydrates contained in the processed liquid nutrient are connatural compared to the textural sensation conferred by all initial carbohydrates contained in the virgin liquid nutrient; and/or—the sweetness conferred by all initial carbohydrates and altered carbohydrates contained in the processed liquid nutrient are connatural compared to the sweetness conferred by all initial carbohydrates contained in the virgin liquid nutrient. Embodiment 37: The method according to any of the preceding embodiments, wherein the processed liquid nutrient is preferably characterized by—a glycemic index which is reduced by at least 5% up to 100%, at least 10% up to 100%, at least 15% up to 100%, at least 20% up to 100%, at least 25% up to 100%, at least 30% up to 100%, at least 35% up to 100%, at least 40% up to 100%, at least 45% up to 100%, at least 50% up to 100%, at least 55% up to 100%, at least 60% up to 100%, at least 65% up to 100%, at least 70% up to 100%, at least 75% up to 100%, at least 80% up to 100%, or reduced by at least 5% up to 90%, at least 10% up to 90%, at least 15% up to 90%, at least 20% up to 90%, at least 25% up to 90%, at least 30% up to 90%, at least 35% up to 90%, at least 40% up to 90%, at least 45% up to 90%, at least 50% up to 90%, at least 55% up to 90%, at least 60% up to 90%, at least 65% up to 90%, at least 70% up to 90% or reduced by at least 5% up to 80%, at least 10% up to 80%, at least 15% up to 80%, at least 20% up to 80%, at least 25% up to 80%, at least 30% up to 80%, at least 35% up to 80%, at least 40% up to 80%, at least 45% up to 80%, at least 50% up to 80%, at least 55% up to 80%, at least 60% up to 80%, or reduced by at least 5% up to 70%, at least 10% up to 70%, at least 15% up to 70%, at least 20% up to 70%, at least 25% up to 70%, at least 30% up to 70%, at least 35% up to 70%, at least 40% up to 70%, at least 45% up to 70%, at least 50% up to 70%; and/or—a calorie count which is reduced by at least 5% up to 100%, at least 10% up to 100%, at least 15% up to 100%, at least 20% up to 100%, at least 25% up to 100%, at least 30% up to 100%, at least 35% up to 100%, at least 40% up to 100%, at least 45% up to 100%, at least 50% up to 100%, at least 55% up to 100%, at least 60% up to 100%, at least 65% up to 100%, at least 70% up to 100%, at least 75% up to 100%, at least 80% up to 100%, or reduced by at least 5% up to 90%, at least 10% up to 90%, at least 15% up to 90%, at least 20% up to 90%, at least 25% up to 90%, at least 30% up to 90%, at least 35% up to 90%, at least 40% up to 90%, at least 45% up to 90%, at least 50% up to 90%, at least 55% up to 90%, at least 60% up to 90%, at least 65% up to 90%, at least 70% up to 90% or reduced by at least 5% up to 80%, at least 10% up to 80%, at least 15% up to 80%, at least 20% up to 80%, at least 25% up to 80%, at least 30% up to 80%, at least 35% up to 80%, at least 40% up to 80%, at least 45% up to 80%, at least 50% up to 80%, at least 55% up to 80%, at least 60% up to 80%, or reduced by at least 5% up to 70%, at least 10% up to 70%, at least 15% up to 70%, at least 20% up to 70%, at least 25% up to 70%, at least 30% up to 70%, at least 35% up to 70%, at least 40% up to 70%, at least 45% up to 70%, at least 50% up to 70%;—in a comparable textural sensation, and preferably in an identical textural sensation; and/or—in a comparable viscosity or viscoelasticity conferred by the carbohydrates, and preferably in an identical viscosity or viscoelasticity conferred by the carbohydrates; and/or—in a comparable crystallinity conferred by the carbohydrates, and preferably in an identical crystallinity conferred by the carbohydrates. Embodiment 38: The method according to any of the preceding embodiments, comprising the steps of (i) providing a virgin liquid nutrient which comprises (a) one or more initial carbohydrates selected from the group consisting of sucrose, inulin, lactose, glucose, galactose, starch, maltose, and fructose; and (b) one or more additional ingredients selected from the group consisting of lipids, proteins, vitamins, metabolites (e.g. organic acids like citric, lactic, oxalic, acetic acids), colloids or colloidal particles, phytochemicals (e.g. carotenoids and polyphenols such as phenolic acids, flavonoids or stilbenes/lignans), fibers, and polysaccharides other than starch; (ii) optionally, adjusting pH value and/or temperature of the virgin liquid nutrient; (iii) optionally, supplementing inorganic phosphate to the virgin liquid nutrient; and (iv) treating the virgin liquid nutrient with one or more enzymes, thereby converting at least a portion of the one or more initial carbohydrates into one or more altered carbohydrates selected from the group consisting of kojibiose, nigerose, trehalose, cellobiose, alpha-D-fructofuranose beta-D-fructofuranose 1,2′:2,3′-dianhydride (DFA III), D-allulose, D-tagatose, isomaltulose, isomaltose, isomalto-oligosaccharides (IMO), gluco-oligosaccharides (GlucOS), and D-mannose; thus obtaining the processed liquid nutrient. Embodiment 39: The method according to any of the preceding embodiments, wherein the total content of said one or more additional ingredients is at least 0.1 wt.-%, preferably at least 0.5 wt.-%, more preferably at least 1.0 wt.-%, relative to the total weight of said virgin liquid nutrient. Embodiment 40: The method according to any of the preceding embodiments, wherein said one or more initial carbohydrates originate from a natural source; and wherein said one or more additional ingredients originate from the same natural source as said one or more initial carbohydrates. Embodiment 41: The method according to any of the preceding embodiments, wherein said virgin liquid nutrient comprises at least three additional ingredients independently of one another selected from the group consisting of lipids, proteins, vitamins, metabolites (e.g. organic acids like citric, lactic, oxalic, acetic acids), colloids or colloidal particles, phytochemicals (e.g. carotenoids and polyphenols such as phenolic acids, flavonoids or stilbenes/lignans), fibers, and polysaccharides other than starch. Embodiment 42: The method according to any of the preceding embodiments, wherein said virgin liquid nutrient is a complex mixture comprising at least 10 different substances including said one or more initial carbohydrates and including said one or more additional ingredients. Embodiment 43: A processed liquid nutrient obtainable by the method according to any of the previous embodiments. Embodiment 44: A processed liquid nutrient manufactured according to any of the previous embodiments that is characterized by—a glycemic index of all initial carbohydrates and altered carbohydrates contained in the processed liquid nutrient, which is lower than the glycemic index of all initial carbohydrates contained in the virgin liquid nutrient; and/or—a calorie count of all initial carbohydrates and altered carbohydrates contained in the processed liquid nutrient, which is lower than the calorie count of all initial carbohydrates contained in the virgin liquid nutrient; and/or—the textural sensation conferred by all initial carbohydrates and altered carbohydrates contained in the processed liquid nutrient are connatural compared to the textural sensation conferred by all initial carbohydrates contained in the virgin liquid nutrient; and/or—the sweetness conferred by all initial carbohydrates and altered carbohydrates contained in the processed liquid nutrient are connatural compared to the sweetness conferred by all initial carbohydrates contained in the virgin liquid nutrient. Embodiment 45: The processed liquid nutrient according to embodiment 43 or 44, wherein the textural sensation conferred by all initial carbohydrates and altered carbohydrates contained in the processed liquid nutrient and the textural sensation conferred by all initial carbohydrates contained in the virgin liquid nutrient are expressed as—the viscosity or viscoelasticity conferred by all the carbohydrates, wherein the viscosity or viscoelasticity conferred by all initial carbohydrates and altered carbohydrates of the processed liquid nutrient in comparison to the viscosity or viscoelasticity conferred by all initial carbohydrates contained in the virgin liquid nutrient is connatural; and/or—the crystallinity conferred by all the carbohydrates, wherein the crystallinity conferred by all initial carbohydrates and altered carbohydrates of the processed liquid nutrient in comparison to the crystallinity conferred by all initial carbohydrates contained in the virgin liquid nutrient is connatural. Embodiment 46: The processed liquid nutrient according to any one of embodiments 43 to 45, wherein the processed liquid nutrient is preferably characterized by—a glycemic index which is reduced by at least 5% up to 100%, at least 10% up to 100%, at least 15% up to 100%, at least 20% up to 100%, at least 25% up to 100%, at least 30% up to 100%, at least 35% up to 100%, at least 40% up to 100%, at least 45% up to 100%, at least 50% up to 100%, at least 55% up to 100%, at least 60% up to 100%, at least 65% up to 100%, at least 70% up to 100%, at least 75% up to 100%, at least 80% up to 100%, or reduced by at least 5% up to 90%, at least 10% up to 90%, at least 15% up to 90%, at least 20% up to 90%, at least 25% up to 90%, at least 30% up to 90%, at least 35% up to 90%, at least 40% up to 90%, at least 45% up to 90%, at least 50% up to 90%, at least 55% up to 90%, at least 60% up to 90%, at least 65% up to 90%, at least 70% up to 90% or reduced by at least 5% up to 80%, at least 10% up to 80%, at least 15% up to 80%, at least 20% up to 80%, at least 25% up to 80%, at least 30% up to 80%, at least 35% up to 80%, at least 40% up to 80%, at least 45% up to 80%, at least 50% up to 80%, at least 55% up to 80%, at least 60% up to 80%, or reduced by at least 5% up to 70%, at least 10% up to 70%, at least 15% up to 70%, at least 20% up to 70%, at least 25% up to 70%, at least 30% up to 70%, at least 35% up to 70%, at least 40% up to 70%, at least 45% up to 70%, at least 50% up to 70%; and/or—a calorie count which is reduced by at least 5% up to 100%, at least 10% up to 100%, at least 15% up to 100%, at least 20% up to 100%, at least 25% up to 100%, at least 30% up to 100%, at least 35% up to 100%, at least 40% up to 100%, at least 45% up to 100%, at least 50% up to 100%, at least 55% up to 100%, at least 60% up to 100%, at least 65% up to 100%, at least 70% up to 100%, at least 75% up to 100%, at least 80% up to 100%, or reduced by at least 5% up to 90%, at least 10% up to 90%, at least 15% up to 90%, at least 20% up to 90%, at least 25% up to 90%, at least 30% up to 90%, at least 35% up to 90%, at least 40% up to 90%, at least 45% up to 90%, at least 50% up to 90%, at least 55% up to 90%, at least 60% up to 90%, at least 65% up to 90%, at least 70% up to 90% or reduced by at least 5% up to 80%, at least 10% up to 80%, at least 15% up to 80%, at least 20% up to 80%, at least 25% up to 80%, at least 30% up to 80%, at least 35% up to 80%, at least 40% up to 80%, at least 45% up to 80%, at least 50% up to 80%, at least 55% up to 80%, at least 60% up to 80%, or reduced by at least 5% up to 70%, at least 10% up to 70%, at least 15% up to 70%, at least 20% up to 70%, at least 25% up to 70%, at least 30% up to 70%, at least 35% up to 70%, at least 40% up to 70%, at least 45% up to 70%, at least 50% up to 70%;—a comparable textural sensation, and preferably by an identical textural sensation; and/or—by a comparable viscosity or viscoelasticity conferred by the carbohydrates, and preferably by an identical viscosity or viscoelasticity conferred by the carbohydrates; and/or—by a comparable crystallinity conferred by the carbohydrates, and preferably by an identical crystallinity conferred by the carbohydrates each and all in comparison to the virgin liquid nutrient. Embodiment 47: The processed liquid nutrient according to any one of embodiments 43 to 46, wherein the processed liquid nutrient is preferably characterized by containing one or more altered carbohydrates selected from the group consisting of—for liquid milk: D-allulose, D-mannose, galactose, glucose, fructose, and D-tagatose, and preferably D-allulose, D-mannose, galactose, glucose, and D-tagatose, and more preferably D-allulose, D-mannose, and D-tagatose, and even more preferably D-allulose, and D-tagatose and most preferably D-allulose; and/or—for extracted fruit juice: nigerose, kojibiose, D-allulose, D-mannose, glucose, fructose cellobiose, trehalose, isomaltulose, and DFA III, and preferably nigerose, kojibiose, D-allulose, D-mannose, glucose, fructose cellobiose, and DFA III, and more preferably nigerose, kojibiose, D-allulose, D-mannose, cellobiose, and DFA III, and most preferably nigerose, kojibiose, and D-allulose and preferably nigerose, kojibiose, D-mannose, D-allulose, DFA III, cellobiose, trehalose, and isomaltulose and; and/or—for a food preparation: DFA III, nigerose, kojibiose, D-allulose, D-tagatose, D-mannose, isomaltulose, cellobiose, trehalose, galactose, glucose, and fructose, and preferably DFA III, nigerose, kojibiose, D-allulose, D-tagatose, D-mannose, isomaltulose, cellobiose, and trehalose, and more preferably DFA III, nigerose, kojibiose, D-allulose, D-tagatose, D-mannose, and isomaltulose, and even more preferably DFA III, nigerose, kojibiose, D-allulose, and D-tagatose, and most preferably DFA III, nigerose, kojibiose, D-allulose, D-tagatose, D-mannose, and isomaltulose, and most preferably DFA III, kojibiose, D-allulose. Embodiment 48: The processed liquid nutrient according to any one of embodiments 43 to 47, wherein the processed liquid nutrient is preferably characterized by containing one or more altered carbohydrates wherein the altered carbohydrate is a disaccharide, selected from the group consisting of—for extracted fruit juice: nigerose, kojibiose, DFA III, cellobiose, trehalose, and isomaltulose, and preferably nigerose, kojibiose, DFA III, cellobiose, and isomaltulose, and more preferably nigerose, kojibiose, cellobiose, and isomaltulose, and even most preferably nigerose, and kojibiose; and/or—for a food preparation: DFA III, nigerose, kojibiose, isomaltulose, cellobiose, and trehalose, and preferably DFA III, nigerose, kojibiose, isomaltulose, and cellobiose, and more preferably DFA III, nigerose, kojibiose, and isomaltulose, and even more preferably DFA III, nigerose, and kojibiose, and most preferably DFA III and kojibiose. Embodiment 49: The processed liquid nutrient according to any one of embodiments 43 to 48, wherein processed liquid nutrient is liquid milk and is characterized by containing one or more altered carbohydrates wherein the altered carbohydrate is D-allulose, D-mannose, galactose, glucose, fructose, and/or D-tagatose and is obtained from an initial carbohydrate at a conversion rate of 5 to 100%, of 5 to 95%, of 5 to 90%, of 5 to 85%, of 5 to 80%, of 5 to 75%, of 5 to 70%, of 5 to 65%, of 5 to 60%, of 5 to 55%, of 5 to 50%, of 5 to 45%, of 5 to 40%, of 5 to 35%, of 5 to 30%, of 5 to 25%, or of 5 to 20%, and preferably at a conversion rate of 10 to 100%, of 10 to 95%, of 10 to 90%, of 10 to 85%, of 10 to 80%, of 10 to 75%, of 10 to 70%, of 10 to 65%, of 10 to 60%, of 10 to 55%, of 10 to 50%, of 10 to 45%, of 10 to 40%, of 10 to 35%, of 10 to 30%, of 10 to 25%, or of 10 to 20%, and more preferably at a conversion rate of 15 to 100%, of 15 to 95%, of 15 to 90%, of 15 to 85%, of 15 to 80%, of 15 to 75%, of 15 to 70%, of 15 to 65%, of 15 to 60%, of 15 to 55%, of 15 to 50%, of 15 to 45%, of 15 to 40%, of 15 to 35%, of 15 to 30%, of 15 to 25%, or of 15 to 20%. Embodiment 50: The processed liquid nutrient according to any one of embodiments 43 to 49, wherein the processed liquid nutrient is extracted fruit juice and is characterized by containing one or more altered carbohydrates wherein the altered carbohydrate is nigerose, kojibiose, D-allulose, cellobiose, trehalose, isomaltulose, DFA III, D-mannose, galactose, fructose, and/or glucose and is obtained from an initial carbohydrate at a conversion rate of 5 to 100%, of 5 to 95%, of 5 to 90%, of 5 to 85%, of 5 to 80%, of 5 to 75%, of 5 to 70%, of 5 to 65%, of 5 to 60%, of 5 to 55%, of 5 to 50%, of 5 to 45%, of 5 to 40%, of 5 to 35%, of 5 to 30%, of 5 to 25%, or of 5 to 20%, and preferably at a conversion rate of 10 to 100%, of 10 to 95%, of 10 to 90%, of 10 to 85%, of 10 to 80%, of 10 to 75%, of 10 to 70%, of 10 to 65%, of 10 to 60%, of 10 to 55%, of 10 to 50%, of 10 to 45%, of 10 to 40%, of 10 to 35%, of 10 to 30%, of 10 to 25%, or of 10 to 20%, and more preferably at a conversion rate of 15 to 100%, of 15 to 95%, of 15 to 90%, of 15 to 85%, of 15 to 80%, of 15 to 75%, of 15 to 70%, of 15 to 65%, of 15 to 60%, of 15 to 55%, of 15 to 50%, of 15 to 45%, of 15 to 40%, of 15 to 35%, of 15 to 30%, of 15 to 25%, or of 15 to 20%. Embodiment 51: The processed liquid nutrient according to any one of embodiments 43 to 50, wherein the processed liquid nutrient is a food preparation and is characterized by containing one or more altered carbohydrates wherein the altered carbohydrate is nigerose, kojibiose, D-allulose, cellobiose, trehalose, isomaltulose, DFA III, D-mannose, D-tagatose, galactose, fructose, and/or glucose and is obtained from an initial carbohydrate at a conversion rate of 5 to 100%, of 5 to 95%, of 5 to 90%, of 5 to 85%, of 5 to 80%, of 5 to 75%, of 5 to 70%, of 5 to 65%, of 5 to 60%, of 5 to 55%, of 5 to 50%, of 5 to 45%, of 5 to 40%, of 5 to 35%, of 5 to 30%, of 5 to 25%, or of 5 to 20%, and preferably at a conversion rate of 10 to 100%, of 10 to 95%, of 10 to 90%, of 10 to 85%, of 10 to 80%, of 10 to 75%, of 10 to 70%, of 10 to 65%, of 10 to 60%, of 10 to 55%, of 10 to 50%, of 10 to 45%, of 10 to 40%, of 10 to 35%, of 10 to 30%, of 10 to 25%, or of 10 to 20%, and more preferably at a conversion rate of 15 to 100%, of 15 to 95%, of 15 to 90%, of 15 to 85%, of 15 to 80%, of 15 to 75%, of 15 to 70%, of 15 to 65%, of 15 to 60%, of 15 to 55%, of 15 to 50%, of 15 to 45%, of 15 to 40%, of 15 to 35%, of 15 to 30%, of 15 to 25%, or of 15 to 20%. Embodiment 52: The processed liquid nutrient according to any one of embodiments 43 to 51, which contains the one or more altered carbohydrates in a concentration of at least, 0.01 wt.-%, or al least 0.03 wt.-%, or al least 0.05 wt.-%, or al least 0.08 wt.-%, or at least 0.1 wt.-%, or al least 0.3 wt.-%, or at least 0.5 wt.-%, or al least 0.8 wt.-%, or at least 1.0 wt.-%, or al least 3.0 wt.-%, or at least 5.0 wt.-%, in each case based on the total weight of all altered carbohydrates and relative to the total weight of the processed liquid nutrient. Embodiment 53: The processed liquid nutrient according to any one of embodiments 43 to 52, which is liquid milk and is characterized by containing one or more altered carbohydrates selected from the group consisting of—D-allulose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.5 wt.-%, of 0.1 to 0.3 wt.-% of 0.1 to 0.1 wt.-%; and/or—D-mannose in a concentration of of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.5 wt.-%, of 0.1 to 0.3 wt.-% of 0.1 to 0.1 wt.-%; and/or—D-tagatose in a concentration of from 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%; in each case relative to the total weight of the liquid milk. Embodiment 54: The processed liquid nutrient according to any one of embodiments 43 to 53, which is a non-concentrated, extracted fruit juice and is characterized by containing one or more altered carbohydrates selected from the group consisting of—D-allulose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2.5 wt.-%, of 0.01 to 2 wt.-%, of 0.01 to 1.5 wt.-%, 0.01 to 1.3 wt.-%, of 0.01 to 1 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2.3 wt.-%, of 0.05 to 2 wt.-%, of 0.05 to 1.5 wt.-%, of 0.05 to 1. wt.-%, of 0.05 to 1 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2.3 wt.-%, of 0.1 to 2 wt.-%, of 0.1 to 1.5 wt.-%, of 0.1 to 1.3 wt.-%, of 0.1 to 1 wt.-%, of 0.1 to 0.5 wt.-%, of 0.1 to 0.3 wt.-% of 0.1 to 0.1 wt.-%; and/or—D-mannose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-% of 0.1 to 0.1 wt.-%; and/or—D-tagatose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%; and/or—nigerose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.6 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.6 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.6 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%; and/or—kojibiose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.6 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.6 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.6 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%; and/or—trehalose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.6 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.6 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.6 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%; and/or—cellobiose in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.6 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.6 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.6 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%; and/or—isomaltulose in a concentration of 0.01 to 20 wt.-%, of 0.01 to 18 wt.-%, of 0.01 to 16 wt.-%, of 0.01 to 14 wt.-%, of 0.01 to 12 wt.-%, of 0.01 to 11 wt.-%, of 0.01 to 10 wt.-%, of 0.01 to 9 wt.-%, of 0.01 to 8 wt.-%, of 0.01 to 7 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.6 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from of 0.05 to 20 wt.-%, of 0.05 to 18 wt.-%, of 0.05 to 16 wt.-%, of 0.05 to 14 wt.-%, of 0.05 to 12 wt.-%, of 0.05 to 11 wt.-%, of 0.05 to 10 wt.-%, of 0.05 to 9 wt.-%, of 0.05 to 8 wt.-%, of 0.05 to 7 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.6 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably of 0.1 to 20 wt.-%, of 0.1 to 18 wt.-%, of 0.1 to 16 wt.-%, of 0.1 to 14 wt.-%, of 0.1 to 12 wt.-%, of 0.1 to 11 wt.-%, of 0.1 to 10 wt.-%, of 0.1 to 9 wt.-%, of 0.1 to 8 wt.-%, of 0.1 to 7 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.6 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.1 wt.-%; and/or—DFA III in a concentration of 0.01 to 10 wt.-%, of 0.01 to 7.5 wt.-%, of 0.01 to 6 wt.-%, of 0.01 to 5.5 wt.-%, of 0.01 to 5 wt.-%, of 0.01 to 4.5 wt.-%, of 0.01 to 4 wt.-%, of 0.01 to 3.6 wt.-%, of 0.01 to 3.5 wt.-%, of 0.01 to 3 wt.-%, of 0.01 to 2.5 wt.-%, of 0.01 to 2 wt.-%, of 0.01 to 1.5 wt.-%, of 0.01 to 1 wt.-%, of 0.01 to 0.8 wt.-%, of 0.01 to 0.5 wt.-%, of 0.01 to 0.3 wt.-%, of 0.01 to 0.1 wt.-%, and preferably from 0.05 to 10 wt.-%, of 0.05 to 7.5 wt.-%, of 0.05 to 6 wt.-%, of 0.05 to 5.5 wt.-%, of 0.05 to 5 wt.-%, of 0.05 to 4.5 wt.-%, of 0.05 to 4 wt.-%, of 0.05 to 3.6 wt.-%, of 0.05 to 3.5 wt.-%, of 0.05 to 3 wt.-%, of 0.05 to 2.5 wt.-%, of 0.05 to 2 wt.-%, of 0.05 to 1.5 wt.-%, of 0.05 to 1 wt.-%, of 0.05 to 0.8 wt.-%, of 0.05 to 0.5 wt.-%, of 0.05 to 0.3 wt.-%, of 0.05 to 0.1 wt.-%, and more preferably 0.1 to 10 wt.-%, of 0.1 to 7.5 wt.-%, of 0.1 to 6 wt.-%, of 0.1 to 5.5 wt.-%, of 0.1 to 5 wt.-%, of 0.1 to 4.5 wt.-%, of 0.1 to 4 wt.-%, of 0.1 to 3.6 wt.-%, of 0.1 to 3.5 wt.-%, of 0.1 to 3 wt.-%, of 0.1 to 2.5 wt.-%, of 0.1 to 2 wt.-%, of 0.1 to 1.5 wt.-%, of 0.1 to 1 wt.-%, of 0.1 to 0.8 wt.-%, of 0.1 to 0.5 wt.-%, of 0.1 to 0.3 wt.-%, of 0.1 to 0.2 wt.-%; in each case relative to the total weight of the non-concentrated, extracted fruit juice. Embodiment 55: The processed liquid nutrient according to any one of embodiments 43 to 54, which is a concentrated extracted fruit juice and is characterized by containing one or more altered carbohydrates selected from the group consisting of—D-allulose in a concentration of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or—D-mannose in a of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or—D-tagatose in a concentration of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or—nigerose in a concentration of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or—kojibiose in a of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or—trehalose in a concentration of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or—cellobiose in a concentration of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or—isomaltulose in a concentration of 0.05 to 90 wt.-%, 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; and/or—DFA III in a concentration of 0.05 to 80 wt.-%, of 0.05 to 70 wt.-%, of 0.05 to 60 wt.-%, of 0.05 to 50 wt.-%, of 0.05 to 40 wt.-%, of 0.05 to 30 wt.-%, of 0.05 to 20 wt.-%, of 0.05 to 10 wt.-%, of 0.05 to 5 wt.-%, of 0.05 to 2.5 wt.-%, of 0.05 to 2 wt.-%, of 0.05 to 1.5 wt.-%, of 0.05 to 1 wt.-%, 0.05 to 0.5 wt.-%, and preferably from of 0.5 to 80 wt.-%, of 0.5 to 70 wt.-%, of 0.5 to 60 wt.-%, of 0.5 to 50 wt.-%, of 0.5 to 40 wt.-%, of 0.5 to 30 wt.-%, of 0.5 to 20 wt.-%, of 0.5 to 10 wt.-%, of 0.5 to 5 wt.-%, of 0.5 to 2.5 wt.-%, of 0.5 to 2 wt.-%, of 0.5 to 1.5 wt.-%, of 0.5 to 1 wt.-%; in each case relative to the total weight of the concentrated extracted fruit juice. Embodiment 56: The processed liquid nutrient according to any one of embodiments 43 to 55, which is a food preparation and is characterized by containing one or more altered carbohydrates selected from the group consisting of—D-allulose in a concentration of 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or—D-mannose in a concentration of 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or—D-tagatose in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or—Nigerose in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or—Kojibiose in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or—Trehalose in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or—Cellobiose in a 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or—Isomaltulose in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; and/or—DFA III in a concentration 0.01 to 40 wt.-%, of 0.01 to 35 wt.-%, of 0.01 to 30 wt.-%, of 0.01 to 25 wt.-%, of 0.01 to 20 wt.-%, of 0.01 to 15 wt.-%, of 0.01 to 10 wt.-%, of 0.01 to 5 wt.-%, of 0.01 to 4 wt.-%, of 0.01 to 3 wt.-%, of 0.01 to 2 wt.-%, of 0.01 to 1 wt.-%, preferably of 0.1 to 40 wt.-%, of 0.1 to 35 wt.-%, of 0.1 to 30 wt.-%, of 0.1 to 25 wt.-%, of 0.1 to 20 wt.-%, of 0.1 to 15 wt.-%, of 0.1 to 10 wt.-%, of 0.1 to 5 wt.-%, of 0.1 to 4 wt.-%, of 0.1 to 3 wt.-%, of 0.1 to 2 wt.-%, of 0.1 to 1 wt.-%, and more preferably of 1 to 40 wt.-%, of 1 to 35 wt.-%, of 1 to 30 wt.-%, of 1 to 25 wt.-%, of 1 to 20 wt.-%, of 1 to 15 wt.-%, of 1 to 10 wt.-%, of 1 to 5 wt.-%, of 1 to 4 wt.-%, of 1 to 3 wt.-%, of 1 to 2 wt.-%, of 1 to 1 wt.-%; in each case relative to the total weight of the food preparation. Embodiment 57: Use of one or more enzymes as defined in any of the preceding embodiments in a method according to any of embodiments 1 to 42 and/or for preparing a processed liquid nutrient according to any of embodiments 43 to 56.

The present invention is further illustrated by the examples and the figures, from which further features, embodiments and advantages may be taken.

### EXAMPLE 1 (LIQUID MILK+BETA-GALACTOSIDASE+GLUCOSE ISOMERASE+PSICOSE EPIMERASE)

One sample is prepared as follows: 10 g of liquid milk is treated with 0.025 wt.-% beta-galactosidase, 0.05 wt.-% glucose isomerase, and 0.05 wt.-% psicose epimerase for 5 hours at 45° C. Subsequently the mixture is heated to 95° C. for 10 minutes to deactivate the enzymes. The lactose, glucose, galactose, fructose and allulose content of the enzyme-free control and inventive samples are analyzed by HPLC. The enzyme-free control liquid milk sample contains 4.7% lactose, no detectable monosaccharides. The inventive liquid milk sample contains 0.5% lactose, 2.1% galactose, 1.0% glucose, 0.8% fructose, and 0.3% allulose.

### EXAMPLE 2 (LIQUID MILK+BETA-GALACTOSIDASE+CELLOBIOSE EPIMERASE+ARABINOSE ISOMERASE)

One sample is prepared as follows: 10 g of liquid milk is treated with 0.025 wt.-% beta-galactosidase, 0.05 wt.-% cellobiose epimerase, and 0.05% arabinose isomerase for 5 hours at 45° C. Subsequently the mixture is heated to 95° C. for 10 minutes to deactivate the enzymes. The lactose, glucose, galactose, mannose and tagatose content of the enzyme-free control and inventive samples are analyzed by HPLC. The enzyme-free control liquid milk sample contains 4.7% lactose, no detectable monosaccharides. The inventive liquid milk sample contains 0.5% lactose, 1.3% galactose, 0.8% tagatose, 1.8% glucose, and 0.3% mannose.

### EXAMPLE 3 (LIQUID MILK+BETA-GALACTOSIDASE+CELLOBIOSE EPIMERASE+ARABINOSE ISOMERASE+GLUCOSE ISOMERASE+PSICOSE ISOMERASE)

One sample is prepared as follows: 10 g of liquid milk is treated with 0.025 wt.-% beta-galactosidase, 0.05 wt.-% cellobiose epimerase, and 0.05 wt.-% arabinose isomerase, 0.05 wt.-% glucose isomerase, and 0.05 wt.-% Psicose epimerase for 5 hours at 45° C. Subsequently the mixture is heated to 95° C. for 10 minutes to deactivate the enzymes. The lactose, glucose, galactose, fructose and allulose content of the enzyme-free control and inventive samples are analyzed by HPLC. The enzyme-free control liquid milk sample contains 4.7% lactose, no detectable monosaccharides. The inventive liquid milk sample contains 0.5% lactose, 1.3% galactose, 0.8% tagatose, 0.8% glucose, 0.2% mannose, 0.8% fructose, and 0.3% allulose.

### EXAMPLE 4 (MANGO JUICE+INVERTASE+CELLOBIOSE EPIMERASE+PSICOSE EPIMERASE)

One sample is prepared as follows: 250 g fresh pulp of Mango fruits are prepared and filtered to separate solid debris. 10 g of the obtained juice are treated with 0.025 wt.-% invertase, 0.05 wt.-% cellobiose epimerase, and 0.05 wt.-% psicose epimerase for 5 hours at 45° C. Subsequently the mixture is heated to 95° C. for 10 minutes to deactivate the enzymes. The sucrose, glucose, fructose, mannose, and allulose content of the enzyme-free control and inventive samples are analyzed by HPLC. The enzyme-free control juice sample contains 10% sucrose, 1% glucose, and 3% fructose. The inventive juice sample contains 1.0% sucrose, 4.7% glucose, 5.2% fructose, 0.8% mannose, and 2.3% allulose.

### EXAMPLE 5 (ORANGE JUICE+SUCROSE PHOSPHORYLASE)

One sample is prepared as follows: 250 g fresh pulp of Orange fruits are prepared and filtered to separate solid debris. 10 g of the obtained juice are treated with 0.5 wt.-% sucrose phosphorylase for 5 hours at 45° C. Subsequently the mixture is heated to 95° C. for 10 minutes to deactivate the enzymes. The sucrose, glucose, fructose, and kojibiose content of the enzyme-free control and inventive samples are analyzed by HPLC. The enzyme-free control juice sample contained 4.2% sucrose, 2.2% glucose, and 2.5% fructose. The inventive juice sample contained 0.5% sucrose, 0.5% glucose, 4.3% fructose, and 3.6% kojibiose.

### EXAMPLE 6 (ORANGE JUICE+SUCROSE PHOSPHORYLASE+TREHALOSE PHOSPHORYLASE)

One sample is prepared as follows: 250 g fresh pulp of Orange fruits is prepared and filtered to separate solid debris. 10 g of the obtained juice are treated with 0.5 wt.-% sucrose phosphorylase, and 0.5 wt.-% trehalose phosphorylase for 5 hours at 45° C. after the addition of inorganic phosphate up to a concentration of 100 mM. Subsequently the mixture is heated to 95° C. for 10 minutes to deactivate the enzymes. The sucrose, glucose, fructose, and trehalose content of the enzyme-free control and inventive samples are analyzed by HPLC. The enzyme-free control juice sample contains 4.2% sucrose, 2.2% glucose, and 2.5% fructose. The inventive juice sample contains 0.5% sucrose, 0.5% glucose, 4.3% fructose, and 3.6% trehalose.

### EXAMPLE 7 (MANGO JUICE+ISOMALTULOSE SYNTHASE)

One sample is prepared as follows: 250 g fresh pulp of mango are prepared and filtered to separate solid debris. 10 g of the obtained juice are treated with 0.5 wt.-% isomaltulose synthase for 5 hours at 45° C. Subsequently the mixture is heated to 95° C. for 10 minutes to deactivate the enzymes. The sucrose, glucose, fructose, and isomaltulose content of the enzyme-free control and inventive samples are analyzed by HPLC. The enzyme-free control juice sample contains 10% sucrose, 1.0% glucose, and 3.0% fructose. The inventive juice sample contains 1.0% sucrose, 1.0% glucose, 3.0% fructose, and 10% isomaltulose.

### EXAMPLE 8 (BANANA JUICE+ISOMALTULOSE SYNTHASE+INULIN FRUCTOFURANOSIDASE)

One sample is prepared as follows: 250 g fresh pulp of banana are prepared and filtered to separate solid debris. 10 g of the obtained juice are treated with 0.5 wt.-% isomaltulose synthase and 0.5 wt.-% inulin fructofuranosidase for 5 hours at 45° C. Subsequently the mixture is heated to 95° C. for 10 minutes to deactivate the enzymes. The sucrose, glucose, fructose, isomaltulose, inulin, and difructose anhydride III (DFA III) content of the enzyme-free control and inventive samples are analyzed by HPLC. The enzyme-free control juice sample contains 6.5% sucrose, 4.2% glucose, 2.7% fructose, and 0.9% inulin. The inventive juice sample contains 0.5% sucrose, 4.2% glucose, 2.7% fructose, 6.0% isomaltulose, 0.3% inulin, and 0.6% DFA III.

### EXAMPLE 9 (MANGO JUICE+ISOMALTULOSE SYNTHASE+D-PSICOSE-3-EPIMERASE)

One sample is prepared as follows: 250 g fresh pulp of mango are prepared and filtered to separate solid debris. 10 g of the obtained juice are treated with 0.05 wt.-% isomaltulose synthase and 0.05 wt.-% D-psicose-3-epimerase for 5 hours at 35° C. Subsequently the mixture is heated to 95° C. for 10 minutes to deactivate the enzymes. The sucrose, glucose, fructose, isomaltulose, and allulose content of the enzyme-free control and inventive samples are analyzed by HPLC. The enzyme-free control juice sample contains 10% sucrose, 1.0% glucose, and 3.0% fructose. The inventive juice sample contains 1.0% sucrose, 9.0% isomaltulose, 1.0% glucose, 2.1% fructose, and 0.9% D-allulose.

### EXAMPLE 10 (ORANGE JUICE+SUCROSE PHOSPHORYLASE+D-PSICOSE-3-EPIMERASE)

One sample is prepared as follows: 250 g fresh pulp of Orange fruits are prepared and filtered to separate solid debris. 10 g of the obtained juice are treated with 0.5 wt.-% sucrose phosphorylase and 0.05 wt.-% D-psicose-3-epimerase for 5 hours at 45° C. Subsequently the mixture is heated to 95° C. for 10 minutes to deactivate the enzymes. The sucrose, glucose, fructose, kojibiose, and allulose content of the enzyme-free control and inventive samples are analyzed by HPLC. The enzyme-free control juice sample contains 4.2% sucrose, 2.2% glucose, and 2.5% fructose. The inventive juice sample contains 0.5% sucrose, 0.5% glucose, 3.0% fructose, 3.6% kojibiose, and 1.3% D-allulose.

### EXAMPLE 11 (APPLE JUICE CONCENTRATE+D-PSICOSE-3-EPIMERASE)

D-psicose-3-epimerase from the organism Burkholderia multivorans with the sequence as being published in the international patent application WO 2016/191267A1 under the SEQ ID NO: 34 was expressed in E. coli. Following cultivation, E. coli cells were harvested by centrifugation and resuspended in lysis buffer containing: 50 mM potassium phosphate buffer pH 6.0, 5 mM MgCl₂, 0.5 mg/ml lysozyme, and 20 U/ml nuclease. The cells were disrupted by repeated freeze-thaw cycles and cell debris was removed by centrifugation. The pH of apple juice concentrate (Apple juice concentrate contains per 100 g product: 65.2 g carbohydrates, of which 61.9 g are sugars, 1.1 g protein, 1.3 g fibers, 0.1 g fat) was adjusted with NaOH to the value of 5.5, 5.0, or 4.5, respectively. The sample was processed by mixing 1 part of D-psicose-3-epimerase preparation with 9 parts of apple juice concentrate adjusted to pH 5.5, 5.0, or 4.5, respectively. The mixture was supplemented with MgCl₂ to a final concentration of 5 mM and incubated for 32 h at 50° C. Subsequently the mixture was heated to 95° C. for 10 minutes to deactivate the enzyme. The sucrose, D-glucose, D-fructose, and D-allulose content of processed apple juice concentrate was analyzed by Ion Chromatography. Table 7 summarizes the sugar composition of the apple juice concentrate before and after treatment with D-psicose-3-epimerase.

TABLE 7
in situ fortification of apple juice concentrate
with D-psicose-3-epimerase

Weight % total sugar

| | | | |
|---|---|---|---|
| Sucrose | Glucose | Fructose | Allulose |

Apple juice adjusted to pH 5.5

| | | | | |
|---|---|---|---|---|
| Before enzymatic | 7.9 | 28.6 | 63.5 | 0 |
| treatment | | | | |
| After enzymatic | 7.6 | 28.5 | 45.0 | 18.9 |
| treatment | | | | |

Apple juice adjusted to pH 5.0

| | | | | |
|---|---|---|---|---|
| Before enzymatic | 8.1 | 28.3 | 63.7 | 0 |
| treatment | | | | |
| After enzymatic | 7.9 | 28.6 | 56.7 | 6.8 |
| treatment | | | | |

Apple juice adjusted to pH 4.5

| | | | | |
|---|---|---|---|---|
| Before enzymatic | 8.1 | 27.8 | 64.0 | 0 |
| treatment | | | | |
| After enzymatic | 8.1 | 29.0 | 4557.3 | 5.7 |
| treatment | | | | |

### EXAMPLE 12 (MANGO JUICE CONCENTRATE+D-PSICOSE-3-EPIMERASE)

D-psicose-3-epimerase from the organism Burkholderia multivorans with the sequence as being published in the international patent application WO 2016/191267A1 under the SEQ ID NO: 34 was prepared as described in Example 11. The pH of mango juice concentrate (Mango juice concentrate contains per 100 g product: 43.7 g carbohydrates, of which 43.7 g are sugars, 2.2 g protein, 2.3 g fibers, 0.9 g fat) was adjusted with NaOH to the value of 5.5, 5.0, or 4.5, respectively. The sample was processed by mixing 1 part of D-psicose-3-epimerase preparation with 9 parts of mango juice concentrate adjusted to 5.5, 5.0, or 4.5, respectively. The mixture was supplemented with MgCl₂ to a final concentration of 5 mM and incubated for 32 h at 50° C. Subsequently the mixture was heated to 95° C. for 10 minutes to deactivate the enzymes. The sucrose, D-glucose, D-fructose, and D-allulose content of the inventive sample was analyzed by Ion Chromatography. The sugar composition of the mango juice concentrate before and after treatment with D-psicose-3-epimerase is reported in Table 8.

TABLE 8
in situ fortification of mango juice concentrate
with D-psicose-3-epimerase

Weight % total sugar

| | | | |
|---|---|---|---|
| Sucrose | Glucose | Fructose | Allulose |

Mango juice adjusted to pH 5.5

| | | | | |
|---|---|---|---|---|
| Before enzymatic | 21.8 | 32.7 | 45.6 | 0 |
| treatment | | | | |
| After enzymatic | 21.7 | 32.7 | 32.6 | 13.0 |
| treatment | | | | |

Mango juice adjusted to pH 5.0

| | | | | |
|---|---|---|---|---|
| Before enzymatic | 21.7 | 32.1 | 45.2 | 0 |
| treatment | | | | |
| After enzymatic | 21.0 | 33.9 | 36.4 | 8.7 |
| treatment | | | | |

Mango juice adjusted to pH 4.5

| | | | | |
|---|---|---|---|---|
| Before enzymatic | 22.0 | 31.5 | 46.5 | 0 |
| treatment | | | | |
| After enzymatic | 22.1 | 32.9 | 38.4 | 6.6 |
| treatment | | | | |

### EXAMPLE 13 (APPLE JUICE CONCENTRATE+SUCROSE PHOSPHORYLASE)

A variant of the Sucrose phosphorylase from Bifidobacterium adolescens as being disclosed as SEQ ID NO:1 in the European Patent EP 3224370 with two substitutions in positions L341I Q345S is expressed in E. coli. Following cultivation, cells are harvested by centrifugation and resuspended in lysis buffer containing: 50 mM MOPS buffer pH 7.0, 2 mM MgCl₂, 0.5 mg/ml lysozyme, and 20 U/ml nuclease. The cells are disrupted by repeated freeze-thaw cycles and cell debris is removed by centrifugation. The pH of apple juice concentrate (Apple juice concentrate contains per 100 g product: 65.2 g carbohydrates, of which 61.9 g are sugars, 1.1 g protein, 1.3 g fibers, 0.1 g fat) is adjusted with NaOH to the value of 5.5, or 4.5, respectively. The sample is processed by mixing 1 part of sucrose phosphorylase preparation with 3 parts of the apple juice concentrate adjusted to pH 5.5 or 4.5, respectively. The mixture is incubated for 33 h at 55° C. Subsequently the mixture is heated to 95° C. for 10 minutes to deactivate the enzyme. The sucrose, glucose, fructose, kojibiose, and maltose content of the enzyme-free control and the inventive samples are analyzed by Ion Chromatography. The sugar composition of the apple juice concentrate before and after treatment with sucrose phosphorylase is reported in Table 9.

TABLE 9
in situ fortification of mango juice concentrate
with Sucrose Prosphorylase

| | Weight % total sugar | | | | |
|---|---|---|---|---|---|
| | Sucrose | Glucose | Fructose | Kojibiose | Maltose |
| | Apple juice adjusted to pH 5.5 | | | | |
| Before enzymatic | 7.8 | 25.3 | 66.9 | 0 | 0 |
| treatment | | | | | |
| After enzymatic | 0.6 | 21.7 | 69.6 | 7.6 | 0.5 |
| treatment | | | | | |
| | Apple juice adjusted to pH 4.5 | | | | |
| Before enzymatic | 7.8 | 25.3 | 66.9 | 0 | 0 |
| treatment | | | | | |
| After enzymatic | 0.9 | 22.0 | 69.2 | 7.4 | 0.5 |
| treatment | | | | | |

### EXAMPLE 14 (MANGO JUICE CONCENTRATE+SUCROSE PHOSPHORYLASE)

A variant of the Sucrose phosphorylase from Bifidobacterium adolescens as being disclosed as SEQ ID NO:1 in the European Patent EP 3224370 with two substitutions in positions L341I Q345S is prepared as described in Example 13. The pH of mango juice concentrate (Mango juice concentrate contains per 100 g product: 43.7 g carbohydrates, of which 43.7 g are sugars, 2.2 g protein, 2.3 g fibers, 0.9 g fat) is adjusted with NaOH to the value of 5.5, or 4.5, respectively. The sample is processed by mixing one part of sucrose phosphorylase preparation with three parts of the mango juice concentrate adjusted to pH 5.5, or 4.5, respectively. The mixture is incubated for 33 h at 55° C. Subsequently the mixture is heated to 95° C. for 10 minutes to deactivate the enzyme. The sucrose, glucose, fructose, kojibiose, and maltose content of the enzyme-free control and inventive samples are analyzed by Ion Chromatography. The sugar composition of the mango juice concentrate before and after treatment with sucrose phosphorylase is reported in Table 108.

TABLE 10
in situ fortification of mango juice concentrate
with sucrose phosphorylase

| | Weight % total sugar | | | | |
|---|---|---|---|---|---|
| | Sucrose | Glucose | Fructose | Kojibiose | Maltose |
| | Mango juice adjusted to pH 5.5 | | | | |
| Before enzymatic | 21.0 | 29.1 | 50.0 | 0 | 0 |
| treatment | | | | | |
| After enzymatic | 0.9 | 21.7 | 59.0 | 17.3 | 1.2 |
| treatment | | | | | |
| | Mango juice adjusted to pH 4.5 | | | | |
| Before enzymatic | 21.0 | 29.1 | 50.0 | 0 | 0 |
| treatment | | | | | |
| After enzymatic | 1.0 | 22.0 | 59.0 | 16.8 | 1.2 |
| treatment | | | | | |

### EXAMPLE 15 (APPLE JUICE CONCENTRATE+D-PSICOSE-3-EPIMERASE+SUCROSE PHOSPHORYLASE)

The D-psicose-3-epimerase from the organism Burkholderia multivorans with the sequence as being published in the international patent application WO 2016/191267A1 under the SEQ ID NO: 34, and a variant of the Sucrose phosphorylase from Bifidobacterium adolescens as being disclosed as SEQ ID NO:1 in the European Patent EP 3224370 with two substitutions in positions L341I Q345S are obtained as described in Examples 11 and 13, respectively. The pH of apple juice concentrate (Apple juice concentrate contains per 100 g product: 65.2 g carbohydrates, of which 61.9 g are sugars, 1.1 g protein, 1.3 g fibers, 0.1 g fat) is adjusted with NaOH to the value of 6.0, 5.5, or 4.0, respectively. The sample is processed by adding one part of D-psicose-3-epimerase preparation and two parts of sucrose phosphorylase preparation with seven parts of the apple juice concentrate adjusted to pH 6.0, 5.5, or 4.0, respectively. The mixture is supplemented with MgCl₂ to a final concentration of 10 mM and incubated for 31 h at 50° C. Subsequently the mixture is heated to 95° C. for 10 minutes to deactivate the enzyme. The sucrose, glucose, fructose, allulose, kojibiose, and maltose content of the processed sample is analyzed by Ion Chromatography. The sugar composition of the apple juice concentrate before and after treatment with D-psicose-3-epimerase and sucrose phosphorylase is reported in Table 11.

TABLE 11
in situ fortification of apple juice concentrate with
D-psicose-3-epimerase and Sucrose phosphorylase

| | Weight % total sugar | | | | | |
|---|---|---|---|---|---|---|
| | Sucrose | Glucose | Fructose | Allulose | Kojibiose | Maltose |
| | Apple juice adjusted to pH 6.0 | | | | | |
| Before | 7.2 | 27.5 | 64.6 | 0 | 0 | 0 |
| enzymatic | | | | | | |
| treatment | | | | | | |
| After | 0.3 | 27.1 | 46.1 | 18.9 | 7.2 | 0.3 |
| enzymatic | | | | | | |
| treatment | | | | | | |
| | Apple juice adjusted to pH 5.5 | | | | | |
| Before | 7.2 | 27.5 | 64.6 | 0 | 0 | 0 |
| enzymatic | | | | | | |
| treatment | | | | | | |
| After | 0.4 | 26.7 | 46.4 | 18.5 | 7.7 | 0.3 |
| enzymatic | | | | | | |
| treatment | | | | | | |
| | Apple juice adjusted to pH 4.0 | | | | | |
| Before | 7.2 | 27.5 | 64.6 | 0 | 0 | 0 |
| enzymatic | | | | | | |
| treatment | | | | | | |
| After | 0.7 | 27.0 | 56.2 | 9.1 | 6.7 | 0.3 |
| enzymatic | | | | | | |
| treatment | | | | | | |

### EXAMPLE 16 (MANGO JUICE CONCENTRATE+D-PSICOSE-3-EPIMERASE+SUCROSE PHOSPHORYLASE)

The D-psicose-3-epimerase from the organism Burkholderia multivorans with the sequence as being published in the international patent application WO 2016/191267A1 under the SEQ ID NO: 34, and a variant of the Sucrose phosphorylase from Bifidobacterium adolescens as being disclosed as SEQ ID NO:1 in the European Patent EP 3224370 with two substitutions in positions L341I Q345S are obtained as described in Examples 11 and 13, respectively. The pH of mango juice concentrate (Mango juice concentrate contains per 100 g product: 43.7 g carbohydrates, of which 43.7 g are sugars, 2.2 g protein, 2.3 g fibers, 0.9 g fat) is adjusted with NaOH to the value of 6.0, 5.5, or 4.0, respectively. The sample is processed by adding one part of D-psicose-3-epimerase preparation and two parts of sucrose phosphorylase preparation with seven parts of the mango juice concentrate adjusted to pH 6.0, 5.5, or 4.0, respectively. The mixture is supplemented with MgCl₂ to a final concentration of 10 mM and incubated for 31 h at 50° C. Subsequently the mixture is heated to 95° C. for 10 minutes to deactivate the enzyme. The sucrose, glucose, fructose, allulose, kojibiose, and maltose content of the processed sample are analyzed by Ion Chromatography. The sugar composition of the apple juice concentrate before and after treatment with D-psicose-3-epimerase and sucrose phosphorylase is reported in Table 12.

TABLE 12
in situ fortification of mango juice concentrate with
D-psicose-3-epimerase and Sucrose Phosphorylase

| | Weight % total sugar | | | | | |
|---|---|---|---|---|---|---|
| | Sucrose | Glucose | Fructose | Allulose | Kojibiose | Maltose |
| | Apple juice adjusted to pH 6.0 | | | | | |
| Before | 20.4 | 31.9 | 47.7 | 0.0 | 0.0 | 0.0 |
| enzymatic | | | | | | |
| treatment | | | | | | |
| After | 0.7 | 27.0 | 39.5 | 14.3 | 17.7 | 0.8 |
| enzymatic | | | | | | |
| treatment | | | | | | |
| | Apple juice adjusted to pH 5.5 | | | | | |
| Before | 20.4 | 31.9 | 47.7 | 0.0 | 0.0 | 0.0 |
| enzymatic | | | | | | |
| treatment | | | | | | |
| After | 0.7 | 26.9 | 39.2 | 14.1 | 18.3 | 0.8 |
| enzymatic | | | | | | |
| treatment | | | | | | |
| | Apple juice adjusted to pH 4.0 | | | | | |
| Before | 20.4 | 31.9 | 47.7 | 0.0 | 0.0 | 0.0 |
| enzymatic | | | | | | |
| treatment | | | | | | |
| After | 0.9 | 27.4 | 43.1 | 10.3 | 17.4 | 0.9 |
| enzymatic | | | | | | |
| treatment | | | | | | |

### EXAMPLE 17 (MILK+FRUCTOSE+D-PSICOSE-3-EPIMERASE)

The D-psicose-3-epimerase from the organism Burkholderia multivorans with the sequence as being published in the international patent application WO 2016/191267A1 under the SEQ ID NO: 34 was obtained as described in Example 11. Fat-reduced ultra-high-temperature (UHT) milk contains per 100 ml: 4.9 g carbohydrates, of which 4.9 g are sugars, 3.5 g protein, 1.5 g fat. The sample was processed by adding one part of D-psicose epimerase preparation and two parts of fructose solution (36% wt) to seven parts of UHT milk. The mixture was supplemented with MgCl₂ to a final concentration of 5.5 mM and incubated for 2 h at 40° C. Subsequently, the mixture was heated to 95° C. for 10 minutes to deactivate the enzyme. The fructose and D-allulose content of the processed samples was analyzed by Ion Chromatography. The fructose to allulose conversion upon the enzymatic treatment with D-psicose epimerase is reported in Table 13.

TABLE 13
in situ fortification of a milk/fructose
mixture with D-psicose-3-epimerase

| | Fructose | Allulose | Conversion |
|---|---|---|---|
| | (g/100 ml) | (g/100 ml) | (%) |
| Before enzymatic | 7.0 | 0.0 | — |
| treatment | | | |
| After enzymatic | 5.1 | 1.9 | 27 |
| treatment | | | |

### EXAMPLE 18 (MILK+HONEY+D-PSICOSE-3-EPIMERASE)

The D-psicose-3-epimerase from the organism Burkholderia multivorans with the sequence as being published in the international patent application WO 2016/191267A1 under the SEQ ID NO: 34 was obtained as described in Example 11. Fat-reduced ultra-high-temperature (UHT) milk contains per 100 ml: 4.9 g carbohydrates, of which 4.9 g are sugars, 3.5 g protein, 1.5 g fat. Honey contains per 100 g: 82 g sugars. Honey was diluted in water to obtain 45% (w/v) solution, which corresponds to 36 g total sugar per 100 ml. The sample was processed by adding one part of D-psicose epimerase preparation and two parts of honey solution to seven parts of UHT milk. The mixture was incubated for 2 h at 40° C. Subsequently, the mixture was heated to 95° C. for 10 minutes to deactivate the enzyme. The fructose and D-allulose content of the processed samples was analyzed by Ion Chromatography. The fructose to allulose conversion upon the enzymatic treatment with D-psicose epimerase is reported in Table 14.

TABLE 14
in situ fortification of a milk/honey
mixture with D-psicose-3-epimerase

| | Fructose | Allulose | Conversion |
|---|---|---|---|
| | (g/100 ml) | (g/100 ml) | (%) |
| Before enzymatic | 3.6 | 0.0 | — |
| treatment | | | |
| After enzymatic | 2.6 | 1.0 | 27 |
| treatment | | | |

### EXAMPLE 19 (MILK+APPLE JUICE CONCENTRATE+D-PSICOSE EPIMERASE)

The D-psicose-3-epimerase from the organism Burkholderia multivorans with the sequence as being published in the international patent application WO 2016/191267A1 under the SEQ ID NO: 34 was obtained as described in Example 11. Fat-reduced ultra-high-temperature (UHT) milk contains per 100 ml: 4.9 g carbohydrates, of which 4.9 g are sugars, 3.5 g protein, 1.5 g fat. Apple juice concentrate (Apple juice concentrate contains per 100 g product: 65.2 g carbohydrates, of which 61.9 g are sugars, 1.1 g protein, 1.3 g fibers, 0.1 g fat) was diluted 1.67-fold in water to obtain a solution which contains 36 g total sugar per 100 ml. The sample was processed by adding one part of D-psicose epimerase preparation and two parts of apple juice concentrate solution to seven parts of UHT milk. The mixture was incubated for 23 h at 40° C. Subsequently, the mixture was heated to 95° C. for 10 minutes to deactivate the enzyme. The fructose and D-allulose content of the processed samples was analyzed by Ion Chromatography. The fructose to allulose conversion upon the enzymatic treatment with D-psicose epimerase is reported in Table 15.

TABLE 15
in situ fortification of a milk/apple juice concentrate mixture
upon enzymatic treatment with D-psicose-3-epimerase

| | Fructose | Allulose | Conversion |
|---|---|---|---|
| | (g/100 ml) | (g/100 ml) | (%) |
| Before enzymatic | 4.5 | 0.0 | — |
| treatment | | | |
| After enzymatic | 3.7 | 0.8 | 17 |
| treatment | | | |

### EXAMPLE 20 (MILK+MANGO JUICE CONCENTRATE+D-PSICOSE EPIMERASE)

The D-psicose-3-epimerase from the organism Burkholderia multivorans with the sequence as being published in the international patent application WO 2016/191267A1 under the SEQ ID NO: 34 was obtained as described in Example 11. Fat-reduced ultra-high-temperature (UHT) milk contains per 100 ml: 4.9 g carbohydrates, of which 4.9 g are sugars, 3.5 g protein, 1.5 g fat. Mango juice concentrate (Mango juice concentrate contains per 100 g product: 43.7 g carbohydrates, of which 43.7 g are sugars, 2.2 g protein, 2.3 g fibers, 0.9 g fat) was diluted 1.25-fold in water to obtain a solution which contains 36 g total sugar per 100 ml. The sample was processed by adding one part of D-psicose epimerase preparation and two parts of mango juice concentrate solution to seven parts of UHT milk. The mixture was supplemented with MgCl₂ to a final concentration of 5 mM and incubated for 23 h at 40° C. Subsequently, the mixture was heated to 95° C. for 10 minutes to deactivate the enzyme. The fructose and D-allulose content of the processed samples was analyzed by Ion Chromatography. The fructose to allulose conversion upon the enzymatic treatment with D-psicose epimerase is reported in Table 16.

TABLE 16
in situ fortification of a milk/mango juice concentrate mixture
upon enzymatic treatment with D-psicose-3-epimerase

| | Fructose | Allulose | Conversion |
|---|---|---|---|
| | (g/100 ml) | (g/100 ml) | (%) |
| Before enzymatic | 3.3 | 0.0 | — |
| treatment | | | |
| After enzymatic | 2.6 | 0.7 | 23 |
| treatment | | | |

### EXAMPLE 21 (YOGURT+FRUCTOSE+D-PSICOSE-3-EPIMERASE)

The D-psicose-3-epimerase from the organism Burkholderia multivorans with the sequence as being published in the international patent application WO 2016/191267A1 under the SEQ ID NO: 34 was obtained as described in Example 11. Yoghurt contains per 100 g: 3.2 g carbohydrates, of which 3.2 g are sugars, 2.2 g protein, 1.2 g fat. The sample was processed by adding one part of D-psicose epimerase preparation and two parts of fructose solution (36% wt) to seven parts of yoghurt. The mixture was supplemented with MgCl₂ to a final concentration of 5.5 mM and incubated for 23 h at 40° C. Subsequently, the mixture was heated to 95° C. for 10 minutes to deactivate the enzyme. The fructose and D-allulose content of the processed samples was analyzed by Ion Chromatography. The fructose to allulose conversion upon the enzymatic treatment with D-psicose epimerase is reported in Table 17.

TABLE 17
in situ fortification of a yogurt/fructose
mixture with D-psicose-3-epimerase

| | Fructose | Allulose | Conversion |
|---|---|---|---|
| | (g/100 ml) | (g/100 ml) | (%) |
| Before enzymatic | 7.0 | 0.0 | — |
| treatment | | | |
| After enzymatic | 5.9 | 1.1 | 16 |
| treatment | | | |

### EXAMPLE 22 (YOGURT+HONEY+D-PSICOSE-3-EPIMERASE)

The D-psicose-3-epimerase from the organism Burkholderia multivorans with the sequence as being published in the international patent application WO 2016/191267A1 under the SEQ ID NO: 34 was obtained as described in Example 11. Yoghurt contains per 100 g: 3.6 g carbohydrates, of which 3.6 g are sugars, 2.5 g protein, 1.3 g fat. Honey contains per 100 g: 82 g sugars. Honey was diluted in water to obtain 45% (w/v) solution, which corresponds to 36 g total sugar per 100 ml. The sample was processed by adding one part of D-psicose epimerase preparation and one part of honey solution to three parts of yoghurt. The mixture was incubated for 23 h at 40° C. Subsequently, the mixture was heated to 95° C. for 10 minutes to deactivate the enzyme. The fructose and D-allulose content of the processed samples was analyzed by Ion Chromatography. The fructose to allulose conversion upon the enzymatic treatment with D-psicose epimerase is reported in Table 18.

TABLE 18
in situ fortification of a yogurt/honey
mixture with D-psicose-3-epimerase

| | Fructose | Allulose | Conversion |
|---|---|---|---|
| | (g/100 ml) | (g/100 ml) | (%) |
| Before enzymatic | 3.6 | 0.0 | — |
| treatment | | | |
| After enzymatic | 3.0 | 0.6 | 16 |
| treatment | | | |

### EXAMPLE 23 (YOGURT+APPLE JUICE CONCENTRATE+D-PSICOSE EPIMERASE)

The D-psicose-3-epimerase from the organism Burkholderia multivorans with the sequence as being published in the international patent application WO 2016/191267A1 under the SEQ ID NO: 34 was obtained as described in Example 11. Yoghurt contains per 100 g: 3.6 g carbohydrates, of which 3.6 g are sugars, 2.5 g protein, 1.3 g fat. Apple juice concentrate (Apple juice concentrate contains per 100 g product: 65.2 g carbohydrates, of which 61.9 g are sugars, 1.1 g protein, 1.3 g fibers, 0.1 g fat) was diluted 1.67-fold in water to obtain a solution which contains 36 g total sugar per 100 ml. The sample was processed by adding one part of D-psicose epimerase preparation and one part of apple juice concentrate solution to three parts of yoghurt. The mixture was incubated for 23 h at 40° C. Subsequently, the mixture was heated to 95° C. for 10 minutes to deactivate the enzyme. The fructose and D-allulose content of the processed samples was analyzed by Ion Chromatography. The fructose to allulose conversion upon the enzymatic treatment with D-psicose epimerase is reported in Table 19.

TABLE 19
in situ fortification of a yogurt/apple juice concentrate
mixture upon enzymatic treatment with D-psicose-3-epimerase

| | Fructose | Allulose | Conversion |
|---|---|---|---|
| | (g/100 ml) | (g/100 ml) | (%) |
| Before enzymatic | 4.5 | 0.0 | — |
| treatment | | | |
| After enzymatic | 4.4 | 0.14 | 3 |
| treatment | | | |

### EXAMPLE 24 (YOGURT+MANGO JUICE CONCENTRATE+D-PSICOSE EPIMERASE)

The D-psicose-3-epimerase from the organism Burkholderia multivorans with the sequence as being published in the international patent application WO 2016/191267A1 under the SEQ ID NO: 34 was obtained as described in Example 11. Yoghurt contains per 100 g: 3.6 g carbohydrates, of which 3.6 g are sugars, 2.5 g protein, 1.3 g fat. Mango juice concentrate (Mango juice concentrate contains per 100 g product: 43.7 g carbohydrates, of which 43.7 g are sugars, 2.2 g protein, 2.3 g fibers, 0.9 g fat) was diluted 1.25-fold in water to obtain a solution which contains 36 g total sugar per 100 ml. The sample was processed by adding one part of D-psicose epimerase preparation and one part of mango juice concentrate solution to three parts of yoghurt. The mixture was supplemented with MgCl₂ to a final concentration of 5 mM and incubated for 23 h at 40° C. Subsequently the mixture was heated to 95° C. for 10 minutes to deactivate the enzyme. The fructose and D-allulose content of the processed samples was analyzed by Ion Chromatography. The fructose to allulose conversion upon the enzymatic treatment with D-psicose epimerase is reported in Table 20.

TABLE 20
in situ fortification of a yogurt/mango juice concentrate
mixture upon enzymatic treatment with D-psicose-3-epimerase

| | Fructose | Allulose | Conversion |
|---|---|---|---|
| | (g/100 ml) | (g/100 ml) | (%) |
| Before enzymatic | 3.3 | 0.0 | — |
| treatment | | | |
| After enzymatic | 3.2 | 0.12 | 4 |
| treatment | | | |

1. A method for preparing an edible processed liquid nutrient by enzymatic in-situ conversion of a virgin liquid nutrient, the method comprising:
(i) providing a virgin liquid nutrient which comprises
one or more initial carbohydrates selected from the group consisting of sucrose, inulin, lactose, glucose, galactose, starch, maltose, and fructose;
one or more additional ingredients selected from the group consisting of lipids, proteins, vitamins, metabolites, colloids or colloidal particles, phytochemicals, fibers, and polysaccharides other than starch;
(ii) optionally, adjusting (ii-a) pH value and/or (ii-b) temperature of the virgin liquid nutrient;
(iii) optionally, supplementing (iii-a) inorganic phosphate and/or (iii-b) an enzyme cofactor and/or (iii-c) one or more initial carbohydrates to the virgin liquid nutrient; and
(iv) treating the virgin liquid nutrient with one or more enzymes,
thereby converting at least a portion of the one or more initial carbohydrates into one or more altered carbohydrates selected from the group consisting of D-allulose, kojibiose, nigerose, trehalose, cellobiose, alpha-D-fructofuranose, beta-D-fructofuranose 1,2′:2,3′-dianhydride (DFA III), D-tagatose, isomaltulose, isomaltose, isomalto-oligosaccharides (IMO), gluco-oligosaccharides (GlucOS), and D-mannose;
thus obtaining the processed liquid nutrient.

2. The method of claim 1, wherein a total content of said one or more additional ingredients is at least 0.1 wt.-%, preferably at least 1 wt.-%, more preferably at least 10.0 wt.-%, still more preferably at least 50.0 wt.-%, yet more preferably at least 70.0 wt.-%, even more preferably at least 80.0 wt.-%, most preferably at least 90.0 wt.-%, and in particular at least 95.0 wt.-%, in each case relative to the total dry weight of said virgin liquid nutrient.

3. The method of claim 1, wherein said one or more initial carbohydrates originate from a natural source; and wherein said one or more additional ingredients originate from the same natural source as said one or more initial carbohydrates.

4. The method of claim 1, wherein said virgin liquid nutrient comprises at least three of the additional ingredients are independently of one another selected from the group consisting of lipids, proteins, vitamins, metabolites, colloids or colloidal particles, phytochemicals, fibers, and polysaccharides other than starch.

5. The method of claim 1, wherein said virgin liquid nutrient is a complex mixture comprising at least 10 different substances including said one or more initial carbohydrates and including said one or more additional ingredients.

6. The method of claim 1, wherein the one or more initial carbohydrates comprise or essentially consist of fructose and wherein the one or more altered carbohydrates comprise or essentially consist of D-allulose or wherein the one or more initial carbohydrates comprise or essentially consist of sucrose and wherein the one or more altered carbohydrates comprise or essentially consist of kojibiose.

7. (canceled)

8. The method of claim 1, wherein the virgin liquid nutrient is selected from the group consisting of liquid milk, extracted fruit juice, and food preparations.

9. The method of claim 1, wherein in step (iv) the treatment of the virgin liquid nutrient into a processed liquid nutrient with the one or more enzymes occurs
in a one-step process upon simultaneous adding of the one or more enzymes and without interim purification of the partially processed liquid nutrient intermediate; or
in a one-step process upon sequential adding of the one or more enzymes and without interim purification of the partially processed liquid nutrient intermediate; or
in a multi-step process upon sequential adding of the one or more enzymes and with interim purification of the partially processed liquid nutrient intermediate.

10. The method of claim 1, wherein the virgin liquid nutrient is treated with one enzyme catalyzing one conversion of one or more initial carbohydrates into one or more altered carbohydrate selected from the group consisting of conversions
initial carbohydrate sucrose into altered carbohydrate kojibiose; and/or
initial carbohydrate sucrose into altered carbohydrate nigerose; and/or
initial carbohydrate fructose into altered carbohydrate D-allulose; and/or
initial carbohydrate glucose into altered carbohydrate D-mannose; and/or
initial carbohydrate fructose into altered carbohydrate D-mannose; and/or
initial carbohydrate inulin into altered carbohydrate DFA III; and/or
initial carbohydrate sucrose into altered carbohydrate isomaltulose; and/or
initial carbohydrate sucrose into altered carbohydrate IMO; and/or
initial carbohydrate sucrose into altered carbohydrate GlucOS; and/or
initial carbohydrate sucrose into altered carbohydrate isomaltose; and/or
initial carbohydrate galactose into altered carbohydrate D-tagatose.

11. The method of claim 1, wherein the virgin liquid nutrient is treated with a first enzyme catalyzing one conversion of one or more initial carbohydrates into one or more first altered carbohydrates, and wherein the one or more first altered carbohydrates are concomitantly treated with one or more additional enzymes catalyzing one or more conversions into one or more second altered carbohydrates selected from the group consisting of conversions
first altered carbohydrate galactose into second altered carbohydrate D-tagatose; and/or
first altered carbohydrate fructose into second altered carbohydrate D-allulose; and/or
first altered carbohydrate glucose into second altered carbohydrate D-mannose; and/or
first altered carbohydrate maltose into second altered carbohydrate IMO; and/or
first altered carbohydrate fructose into second altered carbohydrate D-mannose; and/or
first altered carbohydrate glucose-1-phosphate into second altered carbohydrate cellobiose; and/or
first altered carbohydrate glucose-1-phosphate into second altered carbohydrate cellobiose.

12. The method of claim 1, wherein the virgin liquid nutrient is treated with two enzymes catalyzing the conversion of one initial carbohydrate into two or more altered carbohydrates selected from the group consisting of conversions
initial carbohydrate fructose into altered carbohydrates D-allulose and D-mannose; and/or
initial carbohydrate sucrose into altered carbohydrates fructose and kojibiose; and/or
initial carbohydrate fructose into altered carbohydrates glucose and D-allulose; and/or
initial carbohydrate fructose into altered carbohydrates glucose and D-mannose; and/or
initial carbohydrate lactose into altered carbohydrates galactose and glucose and D-tagatose; and/or—
initial carbohydrate sucrose into altered carbohydrates cellobiose and fructose; and/or
initial carbohydrate sucrose into altered carbohydrates trehalose and fructose; and/or
initial carbohydrate sucrose into altered carbohydrates glucose and D-allulose and fructose; and/or
initial carbohydrate sucrose into altered carbohydrates glucose and D-mannose and fructose; and/or
initial carbohydrate sucrose into altered carbohydrates fructose and nigerose; and/or
initial carbohydrate sucrose into altered carbohydrates IMOs and D-allulose; and/or
initial carbohydrate sucrose into altered carbohydrates IMOS and mannose.

13. The method of claim 1, wherein the virgin liquid nutrient is treated with two enzymes catalyzing the conversion of two or more initial carbohydrates into two or more altered carbohydrates selected from the group consisting of conversions
initial carbohydrates fructose and inulin into altered carbohydrates D-allulose and DFA III; and/or
initial carbohydrates fructose and inulin into altered carbohydrates D-mannose and DFA III; and/or
initial carbohydrates sucrose and inulin into altered carbohydrates isomaltulose and DFA III; and/or
initial carbohydrates sucrose and inulin into altered carbohydrates kojibiose and DFA III; and/or
initial carbohydrates sucrose and inulin into altered carbohydrates nigerose and DFA III; and/or
initial carbohydrates sucrose and fructose into altered carbohydrates isomaltulose and D-allulose; and/or
initial carbohydrates sucrose and fructose into altered carbohydrates kojibiose and D-allulose; and/or
initial carbohydrates sucrose and fructose into altered carbohydrates nigerose and D-allulose; and/or
initial carbohydrates sucrose and fructose into altered carbohydrates isomaltulose and D-mannose; and/or
initial carbohydrates sucrose and fructose into altered carbohydrates kojibiose and D-mannose; and/or
initial carbohydrates sucrose and fructose into altered carbohydrates nigerose and D-mannose; and/or
initial carbohydrates sucrose and inulin into altered carbohydrates IMOs and DFA III; and/or
initial carbohydrates sucrose and glucose into altered carbohydrates isomaltulose and fructose; and/or
initial carbohydrates sucrose and glucose into altered carbohydrates kojibiose and fructose; and/or
initial carbohydrates sucrose and glucose into altered carbohydrates nigerose and fructose; and/or
initial carbohydrates lactose and glucose into altered carbohydrates galactose and D-tagatose; and/or
initial carbohydrates glucose and fructose and inulin into altered carbohydrates D-allulose and DFA III; and/or
initial carbohydrates glucose and fructose and inulin into altered carbohydrates D-mannose and DFA; and/or
initial carbohydrates sucrose and fructose and inulin into altered carbohydrates D-allulose and DFA III; and/or
initial carbohydrates sucrose and fructose and inulin into altered carbohydrates D-mannose and DFA III; and/or
initial carbohydrates sucrose and fructose and inulin into altered carbohydrates isomaltulose and DFA III; and/or
initial carbohydrates sucrose and fructose and inulin into altered carbohydrates kojibiose and DFA III; and/or
initial carbohydrates sucrose and fructose and inulin into altered carbohydrates nigerose and DFA III; and/or
initial carbohydrates sucrose and fructose and inulin into altered carbohydrates isomaltulose and D-allulose; and/or
initial carbohydrates sucrose and fructose and inulin into altered carbohydrates kojibiose and D-allulose; and/or
initial carbohydrates sucrose and fructose and inulin into altered carbohydrates nigerose and D-allulose; and/or
initial carbohydrates sucrose and fructose and inulin into altered carbohydrates isomaltulose and D-mannose; and/or
initial carbohydrates sucrose and fructose and inulin into altered carbohydrates kojibiose and D-mannose; and/or
initial carbohydrates sucrose and fructose and inulin into altered carbohydrates nigerose and D-mannose; and/or
initial carbohydrates sucrose and glucose and inulin into altered carbohydrates isomaltulose and DFA; and/or
initial carbohydrates sucrose and glucose and inulin into altered carbohydrates kojibiose and DFA; and/or
initial carbohydrates sucrose and glucose and inulin into altered carbohydrates nigerose and DFA; and/or
initial carbohydrates sucrose and fructose and glucose into altered carbohydrates isomaltulose and D-allulose; and/or
initial carbohydrates sucrose and fructose and glucose into altered carbohydrates kojibiose and D-allulose; and/or
initial carbohydrates sucrose and fructose and glucose into altered carbohydrates nigerose and D-allulose; and/or
initial carbohydrates sucrose and fructose and glucose into altered carbohydrates isomaltulose and D-mannose; and/or
initial carbohydrates sucrose and fructose and glucose into altered carbohydrates kojibiose and D-mannose; and/or
initial carbohydrates sucrose and fructose and glucose into altered carbohydrates nigerose and D-mannose.

14. The method of claim 1, wherein the processed liquid nutrient is characterized by
a glycemic index of all residual initial carbohydrates and all altered carbohydrates contained in the processed liquid nutrient, which is lower than the glycemic index of all initial carbohydrates contained in the virgin liquid nutrient; and/or
a caloric value of all residual initial carbohydrates and all altered carbohydrates contained in the processed liquid nutrient, which is lower than the caloric value of all initial carbohydrates contained in the virgin liquid nutrient; and/or
a textural sensation conferred by all initial carbohydrates and altered carbohydrates contained in the processed liquid nutrient, which is connatural compared to the textural sensation conferred by all initial carbohydrates contained in the virgin liquid nutrient; and/or
a sweetness conferred by all initial carbohydrates and altered carbohydrates contained in the processed liquid nutrient, which is connatural compared to the sweetness conferred by all initial carbohydrates contained in the virgin liquid nutrient; and/or
a viscosity or viscoelasticity conferred by all initial carbohydrates and altered carbohydrates contained in the processed liquid nutrient, which in comparison to the viscosity or viscoelasticity conferred by all initial carbohydrates contained in the virgin liquid nutrient differs by from 0 to 10%, preferably from 0 to 5%, more preferably from 0 to 2.5%, and most preferably from 0 to 1%; and/or
a crystallinity conferred by all initial carbohydrates and altered carbohydrates contained in the processed liquid nutrient, which in comparison to the crystallinity conferred by all initial carbohydrates contained in the virgin liquid nutrient differs by from 0 to 10%, preferably from 0 to 5%, more preferably from 0 to 2.5%, and most preferably from 0 to 1%.

15. The method of claim 1, wherein the virgin liquid nutrient in (iv) is treated with one or more enzymes characterized by one or more functional features (A), (B), (C), (D), (E):
(A) a catalytic activity for carbohydrate forming in the virgin liquid nutrient of at least 1 to 5000 enzyme units per 100 grams virgin liquid nutrient, at least 25 to 5000 enzyme units per 100 grams virgin liquid nutrient, and preferably about 100 to about 2000 units per 100 grams virgin liquid nutrient;
(B) a high catalytic activity at the pH of the virgin liquid nutrient selected from the group consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, or 9.9; and more preferably selected from the group consisting of 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.0, 7.1, 7.2, 7.3, 7.4, 7.5, and even more preferably selected from the group consisting of pH 5.0 to 7.5, pH 3.0 to pH 6.0, pH 4.0 to 7.0, pH 3.5 to 6.5, pH 4.0 to 6.5, and pH 4.5 to 6.5.
(C) a high process stability in the environment of the virgin liquid nutrient expressed at thermal stability for from 1 hour up to 672 hours, from 1 hour up to 500 hours, from 1 hour up to 400 hours, from 1 hour up to 300 hours, from 1 hour up to 200 hours, from 1 hour up to 168 hours, from 1 hour up to 144 hours, from 1 hour up to 120 hours, from 1 hour up to 96 hours, from 1 hour up to 72 hours, from 1 hour up to 48 hours, from 1 hour up to 24 hours, from 1 hour up to 12 hours, or from 1 hour up to 6 hours;
(D) a high activity at high concentrations of one or more initial carbohydrates of from 0.5 to 70 wt.-%, from 0.5 to 65 wt.-%, from 0.5 to 60 wt.-%, from 0.5 to 55 wt.-%, from 0.5 to 50 wt.-%, from 0.5 to 45 wt.-%, from 0.5 to 40 wt.-%, from 0.5 to 35 wt.-%, from 0.5 to 30 wt.-%, from 0.5 to 25 wt.-%, from 0.5 to 20 wt.-%, or from 0.5 to 15 wt.-%; or from 1 to 70 wt.-%, from 1 to 65 wt.-%, from 1 to 60 wt.-%, from 1 to 55 wt.-%, from 1 to 50 wt.-%, from 1 to 45 wt.-%, from 1 to 40 wt.-%, from 1 to 35 wt.-%, from 1 to 30 wt.-%, from 1 to 25 wt.-%, from 1 to 20 wt.-%, or from 1 to 15 wt.-%; or from 3 to 70 wt.-%, from 3 to 65 wt.-%, from 3 to 60 wt.-%, from 3 to 55 wt.-%, from 3 to 50 wt.-%, from 3 to 45 wt.-%, from 3 to 40 wt.-%, from 3 to 35 wt.-%, from 3 to 30 wt.-%, from 3 to 25 wt.-%, from 3 to 20 wt.-%, or from 3 to 15 wt.-%;
(E) a high activity at high concentrations of one or more altered carbohydrates of from 5 to 70 wt.-%, from 5 to 65 wt.-%, from 5 to 60 wt.-%, from 5 to 55 wt.-%, from 5 to 50 wt.-%, from 5 to 45 wt.-%, from 5 to 40 wt.-%, from 5 to 35 wt.-%, from 5 to 30 wt.-%, from 5 to 25 wt.-%, from 5 to 20 wt.-%, or from 5 to 15 wt.-%; or from 10 to 70 wt.-%, from 10 to 65 wt.-%, from 10 to 60 wt.-%, from 10 to 55 wt.-%, from 10 to 50 wt.-%, from 10 to 45 wt.-%, from 10 to 40 wt.-%, from 10 to 35 wt.-%, from 10 to 30 wt.-%, from 10 to 25 wt.-%, from 10 to 20 wt.-%, or from 10 to 15 wt.-%; or from 15 to 70 wt.-%, from 15 to 65 wt.-%, from 15 to 60 wt.-%, from 15 to 55 wt.-%, from 15 to 50 wt.-%, from 15 to 45 wt.-%, from 15 to 40 wt.-%, from 15 to 35 wt.-%, from 15 to 30 wt.-%, from 15 to 25 wt.-%, or from 15 to 20 wt.-%.

16. The method of claim 1, wherein after (iv) the one or more enzymes that were employed for treatment of the virgin liquid nutrient and are contained in the processed liquid nutrient are inactivated; preferably
a) by heat treatment of the processed liquid nutrient;
b) by shifting the pH of the processed liquid nutrient;
c) by treatment of the processed liquid nutrient with protease enzymes; and/or
d) by supplementation of inhibitory chemical substances, preferably mineral salts, into the processed liquid nutrient.

17. The method of claim 1, wherein after (iv) the one or more enzymes that were employed for treatment of the virgin liquid nutrient are not inactivated and remain in the processed liquid nutrient, or
wherein the one or more enzymes for treatment of the virgin liquid nutrient in (iv) are not immobilized, or wherein the method does not involve adjusting the pH value of the virgin liquid nutrient, and wherein the virgin liquid nutrient is optionally liquid milk.

18. (canceled)

19. (canceled)

20. The method of claim 1, wherein in (ii) the pH value of the virgin liquid nutrient is adjusted to pH values of not more than pH 6.5, or not more than pH 6.0, or not more than pH 5.5, or not more than pH 5.0, or not more than pH 4.5, or not more than pH 4.0, or not more than pH 3.5.

21. The method of claim 1, wherein the processed liquid nutrient is provided as foodstuff without further processing or wherein the one or more enzymes for treatment of the virgin liquid nutrient in (iv) are not immobilized.

22. The method of claim 1, wherein
the virgin liquid nutrient comprises liquid milk, wherein (iii) involves supplementing (iii-c) fructose as initial carbohydrate, and wherein (iv) involves the enzymatic conversion of at least a portion of the fructose into D-allulose or
the virgin liquid nutrient comprises a mixture of liquid milk and extracted fruit juice, wherein said mixture contains fructose as initial carbohydrate, and wherein (iv) involves the enzymatic conversion of at least a portion of the fructose into D-allulose, or
the virgin liquid nutrient comprises a mixture of liquid milk and a food preparation, wherein said mixture contains fructose as initial carbohydrate, and wherein (iv) involves the enzymatic conversion of at least a portion of the fructose into D-allulose, and wherein preferably the food preparation is honey, or
the virgin liquid nutrient comprises an extracted fruit juice containing sucrose as initial carbohydrate, and wherein (iv) involves the enzymatic conversion of at least a portion of the sucrose into kojibiose, or
the virgin liquid nutrient comprises an extracted fruit juice containing fructose as initial carbohydrate, and wherein step (iv) involves the enzymatic conversion of at least a portion of the fructose into D-allulose, or
the virgin liquid nutrient comprises an extracted fruit juice, containing sucrose and fructose as initial carbohydrates, and wherein step (iv) involves the enzymatic conversion of at least a portion of the sucrose into kojibiose and at least a portion of fructose into D-allulose.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. The processed liquid nutrient containing one or more altered carbohydrates selected from the group consisting of
for liquid milk: D-allulose, D-mannose, galactose, glucose, fructose, and D-tagatose, and preferably D-allulose, D-mannose, galactose, glucose, and D-tagatose, and more preferably D-allulose, D-mannose, and D-tagatose, and even more preferably D-allulose, and D-tagatose and most preferably D-allulose; and/or
for extracted fruit juice: nigerose, kojibiose, D-allulose, D-mannose, glucose, fructose, cellobiose, trehalose, isomaltulose, IMO, GlucOS, isomaltose, and DFA III, and preferably nigerose, kojibiose, D-allulose, D-mannose, glucose, IMO, GlucOS, isomaltose, fructose, cellobiose, and DFA III, and more preferably nigerose, kojibiose, D-allulose, D-mannose, cellobiose, and DFA III, and most preferably nigerose, kojibiose, and D-allulose and preferably nigerose, kojibiose, D-mannose, D-allulose, DFA III, cellobiose, trehalose, and isomaltulose and; and/or
for a food preparation: DFA III, nigerose, kojibiose, D-allulose, D-tagatose, D-mannose, isomaltulose, cellobiose, trehalose, galactose, glucose, fructose, IMO, GlucOS, isomaltose, and preferably DFA III, nigerose, kojibiose, D-allulose, D-tagatose, D-mannose, isomaltulose, IMO, GlucOS, isomaltose, cellobiose, and trehalose, and more preferably DFA III, nigerose, kojibiose, D-allulose, D-tagatose, D-mannose, and isomaltulose, and even more preferably DFA III, nigerose, kojibiose, D-allulose, and D-tagatose, and most preferably DFA III, nigerose, kojibiose, D-allulose, D-tagatose, D-mannose, and isomaltulose, and most preferably DFA III, kojibiose, D-allulose.

35. The processed liquid nutrient according to claim 33 or 34, which contains the one or more altered carbohydrates in a concentration of at least, 0.01 wt.-%, or at least 0.03 wt.-%, or at least 0.05 wt.-%, or at least 0.08 wt.-%, or at least 0.1 wt.-%, or at least 0.3 wt.-%, or at least 0.5 wt.-%, or at least 0.8 wt.-%, or at least 1.0 wt.-%, or at least 3.0 wt.-%, or at least 5.0 wt.-%, in each case based on the total weight of all altered carbohydrates and relative to the total weight of the processed liquid nutrient.
